SEQUENCE LISTING (26 sequences)

SEQ ID NO: 1 - protein, 121 aa, synthetic construct
QVQLVQSGAEVKKPGASVKLSCKASGYTFTDYYIHWVRQAPGQGLEWIGYINPNSGYTNYAQKFQGRATMTADKSINTAYVELSRLRSDDTAVYFCTRYMWERVTGFFDFWGQGTMVTVSS

SEQ ID NO: 2 - protein, 108 aa, synthetic construct
DIQMTQSPSSVSASVGDRVTITCLASEDISDDLAWYQQKPGKAPKVLVYTTSSLQSGVPSRFSGSGSGTDFTLTISSLQPEDFATYFCQQTYKFPPTFGGGTKVEIKR

SEQ ID NO: 3 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 4 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 5 - protein, 5 aa, synthetic construct
DYYIH

SEQ ID NO: 6 - protein, 17 aa, synthetic construct
YINPNSGYTNYAQKFQG

SEQ ID NO: 7 - protein, 12 aa, synthetic construct
YMWERVTGFFDF

SEQ ID NO: 8 - protein, 11 aa, synthetic construct
LASEDISDDLA

SEQ ID NO: 9 - protein, 7 aa, synthetic construct
TTSSLQS

SEQ ID NO: 10 - protein, 9 aa, synthetic construct
QQTYKFPPT

SEQ ID NO: 11 - protein, 5 aa, synthetic construct
DYGVS

SEQ ID NO: 12 - protein, 16 aa, synthetic construct
VIWGSETTYYNSALKS

SEQ ID NO: 13 - protein, 12 aa, synthetic construct
HYYYGGSYAMDY

SEQ ID NO: 14 - protein, 11 aa, synthetic construct
RASQDISKYLN

SEQ ID NO: 15 - protein, 7 aa, synthetic construct
HTSRLHS

SEQ ID NO: 16 - protein, 9 aa, synthetic construct
QQGNTLPYT

SEQ ID NO: 17 - protein, 45 aa, synthetic construct
TTTPAPRPPTPAPTIASQPLSLRPEACRPAAGGAVHTRGLDFACD

SEQ ID NO: 18 - protein, 49 aa, synthetic construct
KPTTTPAPRPPTPAPTIASQPLSLRPEASRPAAGGAVHTRGLDFASDKP

SEQ ID NO: 19 - protein, 47 aa, synthetic construct
SGTTTPAPRPPTPAPTIASQPLSLRPEACRPAAGGAVHTRGLDFACD

SEQ ID NO: 20 - protein, 24 aa, synthetic construct
IYIWAPLAGTCGVLLLSLVITLYC

SEQ ID NO: 21 - protein, 42 aa, synthetic construct
KRGRKKLLYIFKQPFMRPVQTTQEEDGCSCRFPEEEEGGCEL

SEQ ID NO: 22 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 5 aa, synthetic construct
GGGGS

SEQ ID NO: 24 - protein, 18 aa, synthetic construct
GSTSGSGKPGSGEGSTKG

SEQ ID NO: 25 - protein, 21 aa, synthetic construct
MALPVTALLLPLALLLHAARP

SEQ ID NO: 26 - protein, 730 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSQVQLVQSGAEVKKPGASVKLSCKASGYTFTDYYIHWVRQAPGQGLEWIGYINPNSGYTNYAQKFQGRATMTADKSINTAYVELSRLRSDDTAVYFCTRYMWERVTGFFDFWGQGTMVTVSSGSTSGSGKPGSGEGSTKGDIQMTQSPSSVSASVGDRVTITCLASEDISDDLAWYQQKPGKAPKVLVYTTSSLQSGVPSRFSGSGSGTDFTLTISSLQPEDFATYFCQQTYKFPPTFGGGTKVEIKRGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0064855 A1
(43) Pub. Date: Feb. 27, 2025

(54) Use of dual-target CAR-T cells in treating B-cell autoimmune diseases

(71) Applicant: Gracell Biotechnologies (Shanghai) Co., Ltd., Shanghai (CN)

(72) Inventors: Huan Shi, Shanghai (CN); Lianjun Shen, Shanghai (CN); Xiaoyan Jiang, Shanghai (CN); Wei Cao, Shanghai (CN)

(21) Appl. No.: 18/799,135

(22) Filed: Aug. 9, 2024

Related U.S. Application Data
(63) Continuation of application No. PCT/CN2024/093376, filed on May 15, 2024.

(30) Foreign Application Priority Data
Aug. 10, 2023 (CN) 202311005978.2

(51) Int. Cl. A61K 35/17; A61K 39/00; C07K 14/705; C07K 14/725; C07K 16/28; C12N 5/0783

(52) U.S. Cl. CPC A61K 35/17; A61K 39/4611; A61K 39/4631; A61K 39/464412; A61K 39/464417; C07K 14/7051; C07K 14/70517; C07K 14/70578; C07K 16/2803; C07K 16/2878; C12N 5/0636; A61K 2239/13; A61K 2239/21; C07K 2317/31; C07K 2317/565; C07K 2317/622; C07K 2319/02; C07K 2319/03

(57) ABSTRACT

A bispecific or dual target CAR or CAR-T cells can be used to treat and/or prevent an autoimmune disease. The bispecific CAR may target CD19 and BCMA. The CAR may have a loop structure, which is shown as follow: L-VL, CD19-VH, BCMA-VL, BCMA-VH, CD19-H-TM-C-CD3ζ; the BCMA-CD19 CAR-T cells transduced by the chimeric antigen receptor targeting CD19 and BCMA can completely block the pathway of producing autoantibodies, and completely cover the B cells, plasmablasts and plasma cells at multiple differentiation stages that produce autoantibodies, so that the dual-target CAR-T therapy designed based on this concept can be aimed at refractory SLE and is expected to achieve faster remission and deeper and lasting curative effect.

### RELATED APPLICATIONS

This application claims priority to Chinese patent application number 202311005978.2, filed on Aug. 10, 2023, and international application number PCT/CN2024/093376, filed on May 15, 2024. The content of each of these applications is incorporated herein by reference in its entirety.

### SEQUENCE LISTING

The present application contains a computer-readable sequence listing that is submitted herewith electronically in XML. The sequence listing is entitled, “GC012F-500-WO-PCT2.XML”, is 24,857 bytes in size, and was created on Aug. 5, 2024. The sequence listing is incorporated herein by reference in its entirety.

### FIELD OF THE INVENTION

CAR-T cells may be used in the treatment or prevention of a B-cell autoimmune disease. In specific embodiments, a dual-target or bispecific CAR may be used to treat an autoimmune disease such as systemic lupus erythematosus, myositis, scleroderma or systemic sclerosis (SSc), myasthenia gravis, and nephritis.

### BACKGROUND OF THE INVENTION

The immune system must first identify foreign substances or dangerous substances in order to protect the body from foreign substances or dangerous substances. These substances include bacteria, viruses, parasites (such as worms), some cancer cells, and even transplanted organs and tissues. These substances have molecules that can be recognized by the immune system and can stimulate the immune system's response. These molecules are called antigens. Antigens can be contained in cells or on the surface of cells (such as bacteria or cancer cells) or components of viruses.

When some white blood cells (B cells and T cells) encounter antigens, they will learn how to attack antigens, thus protecting the body from potentially dangerous antigens. B cells produce antibodies, which are one of the main immune defense mechanisms of human body against antigens. Antibodies bind closely to specific antigens and label them for attack or direct neutralization. The body produces thousands of different kinds of antibodies. Each antibody is specific for a specific antigen. Cells in the immune system will remember specific antigens so that they can attack more effectively the next time they encounter them.

Cells in people's own tissues also have antigens. Under normal circumstances, the immune system only reacts to foreign or dangerous substances, but not to the antigens of its own tissues. However, sometimes immune dysfunction occurs, treating one's own tissue as foreign, producing antibodies (called autoantibodies) or immune cells attacking one's own cells or tissues. This response is called autoimmune response. It leads to inflammation and tissue damage. This response may lead to autoimmune diseases.

At present, many autoimmune diseases have been found, which can be systemic, such as systemic lupus erythematosus, which will affect the skin, joints, kidneys and central nervous system; they can also be organic, such as type 1 diabetes, which mainly affects the pancreatic health of the body. The loss of tolerance of B cells or T cells is often related to autoimmunity. In fact, human leukocyte antigen (HLA) locus is usually directly related to the increased risk of autoimmune diseases.

At present, immunomodulatory drugs used for autoimmune diseases are broad-ranging and non-disease-specific, and usually cause side effects such as infection and malignant diseases. Obviously, the response of most patients to these treatments is not ideal, or even there is no response at all. Therefore, it is urgent for researchers to develop new drugs or re-positioned drugs through molecular and clinical research on patients with special autoimmune diseases in combination with high-throughput integrated database analysis. Also, individualized drugs and new diagnostic methods complement each other, thus helping researchers to better analyze the pathogenesis of autoimmune diseases, and meanwhile researchers need to conduct more patient-centered related clinical trials. Prevention of autoimmune diseases should also be a part of early intervention. In this article, researchers interpret different types of current therapies, such as synthetic drugs, cell therapy, targeted metabolic pathways and microbiomes and other strategies, and also emphasize the dynamic combination of basic research, new technologies and clinical trials, thus helping to understand more about the pathogenesis of autoimmune diseases and develop new therapies.

Nowadays, researchers are more and more aware of the complexity of autoimmune diseases, but the current treatment is only based on a simplified and reductionist pathological understanding and knowledge; in clinical practice, patients are often treated with incrementally reactive, trial-and-error approaches starting with different conventional disease-modifying drugs, such as methotrexate for rheumatoid arthritis, interferon B for the treatment of multiple sclerosis, and corticosteroids for the treatment of inflammatory bowel disease and psoriasis. If patients do not respond adequately, researchers will upgrade new drugs, such as JAK inhibitors or biological drugs. All of these drugs have a wide spectrum of effects, but they are not disease-specific and often have certain side effects. Compared with individualized therapies that have entered the field of oncology research, these drugs may lack individualized characteristics. Therefore, in order to achieve more specific and individualized treatment for patients with autoimmune diseases, researchers need to understand the complexity of individual autoimmune diseases and the manifestations of diseases in different patients.

Systemic lupus erythematosus (SLE) is an autoimmune inflammatory connective tissue disease involving multiple organs with complex pathogenesis, which is mainly related to the mistaken recognition of endogenous antigens by the immune system, the emergence of autoreactive B cells and the production of autoantibodies. The onset age of SLE is mostly 20-40 years old with obvious gender tendency, with the ratio of male to female being about 1:9-12. According to the literature statistics, the global prevalence rate of SLE is about 43.7/100,000 people/year, with great differences in different regions:it is about 3.7-4.9/100,000 people/year in North America, about 1.5-7.4/100,000 people/year in Europe, about 15.9/100,000 people/year in Central Asia, and about 110.82/100,000 people/year in tropical Latin America. The prevalence rate in China is about 47.53/100,000 people/year, and the total number of patients is about 700,000.

SLE is an immune-mediated systemic disease, which can cause various abnormalities in the skin, kidney, blood and musculoskeletal system. The clinical symptoms of SLE are heterogeneous, with great individual differences. Common symptoms of systemic lupus erythematosus may include joint pain and arthritis, buccal rash and other rashes, pleurisy or pericarditis, kidney or central nervous system involvement, and blood system hemocytopenia. The manifestations of systemic lupus erythematosus are changeable, and acute recurrence and remission of the disease often occur alternately in the course of the disease. The severity of SLE varies greatly, which may bring great challenges due to accumulated organ damage and coagulation defects, and may be fatal according to severity of organ involvement.

### Treatment Status of SLE

At present, long-term use of hydroxychloroquine (HCQ) as the basic treatment is recommended for SLE, in addition to non-steroidal anti-inflammatory drugs, low-dose hormones and immunosuppressants. Cyclophosphamide can be used as an initial treatment for severe SLE or as a rescue treatment when other drugs have failed. However, there are still a small number of critically ill or refractory patients with poor response to the above traditional drug treatment and poor prognosis, and the adverse reactions related to non-selective immunosuppressants, such as infection and metabolic abnormalities, are gradually increasing with the extension of medication time. On the basis of traditional treatment, immune targeted therapy is also increasingly used in clinic. However, traditional immunosuppressants, such as dexamethasone, cyclophosphamide and anti-CD20-specific antibodies, can clear short-lived plasmablasts and plasma cells, but they cannot completely clear antibody-secreting cells (ASCs) secreting autoantibodies, including long-lived plasma cells (LLPCs), which leads to recurrent SLE, thus failing to achieve long-term significant effect in clinic. To sum up, it is particularly important to improve the treatment of systemic lupus erythematosus.

### Reasons for Target Selection for the Treatment of SLE by CAR-T Cells

B cells play a central role in the pathogenesis of SLE through antibody-dependent and non-antibody-dependent functions.

The lack of self-tolerance during the development of B cells leads to the occurrence of autoimmunity and the production of autoantibodies. Antinuclear antibody (ANA) is an autoantibody against DNA, RNA, proteins or molecular complexes of these substances in the nucleus, and it is the main serological marker of SLE. ANA has long been found in the diagnosis of lupus, and its role in SLE has been paid more and more attention. First of all, the hematological symptoms of many autoimmune diseases are directly driven by autoantibodies, such as Coombs positive (anti-human globulin or red blood cells) hemolytic anemia and immune platelet deficiency. Secondly, an inherent histopathological feature of lupus nephritis is the significant presence of a glomerular immune complex composed of many subtypes of immunoglobulin. In 1960s, anti-dsDNA antibody was eluted from the kidney of lupus nephritis. In addition, studies have shown that a variety of autoantibodies in addition to anti-dsDNA can be eluted from the kidney of lupus nephritis. Finally, the direct evidence that lupus autoantibodies can cause systemic inflammation comes from neonatal lupus erythematosus (NLE), and passive transfer of maternal autoantibodies across the placenta will cause NLE.

The types of ANAs in SLE patients mainly include anti-DNA antibodies and anti-RNA-binding protein antibodies (anti-RBPs). Anti-DNA antibodies are mainly expressed and secreted by naïve B cells that have transited to plasmablasts, and their serum titers will change significantly with the progress of the disease, while anti-RBP antibodies are mainly secreted by LLPC, and generally do not change significantly with the progress of the disease. It has been reported that the antibody titer of SLE susceptible mice is mainly maintained by LLPC. Among them, anti-DNA antibodies can form immune complexes with target antigens, leading to renal deposition, complement activation and cytokine production. Free anti-DNA antibodies can also cross-bind to receptors in neurons to cause nervous system symptoms or directly bind to antigens in kidneys to produce pathological reactions, while anti-RBP antibodies can also form immune complexes, leading to the production of cytokines and possible renal deposition and complement activation.

In the early 1990s, it was found that B cells not only directly produce pathological autoantibodies in SLE, but also activate lupus-related T cells through cytokines presented and secreted by antigens. First of all, in MRL/lpr lupus model mice, mice with B cells removed will show weakened T cell activation and reduced interstitial nephritis, which can completely prevent the progress of SLE disease. It is particularly noteworthy that this phenomenon can't be completely explained by the lack of autoantibodies, because when B cells that can't secrete immunoglobulin are replanted into the model mice with B cell cleared, the inflammatory T cell foci in the animals will reappear, and the mice will become susceptible mice again. These studies show the presence of antibody-independent B cell functions, including antigen presentation and cytokines. Secondly, in MRL/lpr mice lacking CD11c+ dendritic cells, T cells can still be activated. Nevertheless, when the antigen presenting ability of B cells is deleted at the gene level, the activation of CD4+ T cells, the differentiation of effector memory T and follicular helper T cell (Tfh) and the progress of autoimmunity will be prevented.

In view of the important role of B cells and plasma cells in the pathogenesis of SLE, the current research on targeted therapy for SLE mainly focuses on targets related to B cells and plasma cells, such as B lymphocyte stimulator BAFF/BLyS, proliferation-inducing ligands APRIL, CD20, CD22, CD19 and so on. Other targets include FcγRIIb, Bruton tyrosine kinase, proteasome, T cell related targets, macrophage related targets, intracellular signal molecules, costimulatory factors, IgE and intestinal flora. See Table 1 for details.

TABLE 1
Some targeted therapeutic products for SLE that
have been marketed or are under research.

| | | | | | Development and |
|---|---|---|---|---|---|
| No. | Drug name | Drug type | Target | Target's role | research phase |
| 1 | Belimumab | Monoclonal | BLyS | It activates | Commercially |
| | | antibody | | downstream Akt | available |
| 2 | Tabalumab | Monoclonal | | pathway, improves | In clinical trials |
| | | antibody | | the metabolic | |
| 3 | Blisibimod | Monoclonal | | activity of B cells, | In clinical trials |
| | | antibody | | promotes the | |
| 4 | Atacicept | Fusion protein | | proliferation and | In clinical trials |
| 5 | Telitacicept | Fusion protein | | activation of B | Commercially |
| | | (dual target) | | cells, and prevents | available |
| | | | | B cells from | |
| | | | | apoptosis. | |
| | | | APRIL | It mainly acts on | |
| | | | | the late activation | |
| | | | | and antibody | |
| | | | | secretion stage | |
| | | | | of B cells, and | |
| | | | | regulates the | |
| | | | | differentiation | |
| | | | | and proliferation | |
| | | | | of B cells. | |
| 6 | Rituximab | Monoclonal | CD20 | It is only expressed | Commercially |
| | | antibody | | in pre-B cells to | available |
| 7 | Ocrelizumab | Monoclonal | | memory B cells, can | Commercially |
| | | antibody | | affect the activation, | available |
| 8 | Obinutuzumab | Monoclonal | | proliferation, | In clinical trials |
| | | antibody | | differentiation and | |
| 9 | Ofatumumab | Monoclonal | | signal transduction | In clinical trials |
| | | antibody | | of B cells. | |
| 10 | Veltuzumab | Monoclonal | | | In clinical trials |
| | | antibody | | | |
| 11 | Obexelimab | Monoclonal | CD19 | As a functional | In clinical trials |
| | | antibody | | receptor, it is | |
| | | | | widely expressed | |
| | | | | in B cells, it is | |
| | | | | closely related to | |
| | | | | the activation, | |
| | | | | signal transduction | |
| | | | | and growth of B | |
| | | | | cells, and | |
| | | | | participates in | |
| | | | | the transport of | |
| | | | | Ca²⁺ in B cells. | |
| 12 | Epratuzumab | Monoclonal | CD22 | It is expressed | In clinical trials |
| | | antibody | | on the surface of | |
| | | | | mature B cells in | |
| | | | | a restricted | |
| | | | | manner, and is | |
| | | | | closely related to | |
| | | | | the differentiation, | |
| | | | | maturation and | |
| | | | | migration of B cells. | |
| 13 | Anifrolumab | Monoclonal | IFN-I | It plays an | Commercially |
| | | antibody | | important role in | available |
| 14 | Sifalimumab | Monoclonal | | the pathogenes is | In clinical trials |
| | | antibody | | of SLE, especially | |
| 15 | Rontalizumab | Monoclonal | | IFN-α, whose serum | In clinical trials |
| | | antibody | | concentration is | |
| | | | | positively correlated | |
| | | | | with SLE activity. | |
| 16 | Ruplizumab | Monoclonal | CD40L | It interacts with | In clinical trials |
| | | antibody | | CD40 on B cells, | |
| 17 | Toralizumab | Monoclonal | | and is very | In clinical trials |
| | | antibody | | important for the | |
| | | | | growth, | |
| | | | | differentiation | |
| | | | | and activation | |
| | | | | of B cells. | |

The existing B-cell targeted therapy directly targets B-cell surface markers BLyS, APRIL, CD19, CD20 and CD22 etc., and achieves its pharmacological effects by regulating the activation, proliferation, differentiation and apoptosis of B-cells and/or plasma cells. Belimumab targeting BLyS is the first biological drug approved for the treatment of SLE. The main drugs targeting CD20 include rituximab and ocrelizumab, and the 2019 EULAR Guidelines and the 2020 China SLE Diagnosis and Treatment Guidelines all suggest that rituximab can be selected for patients with refractory SLE-related severe thrombocytopenia. However, the targeted therapy products that are commercially available have not achieved significant therapeutic effect or long-term drug remission in SLE. According to a statistic, 26 recurrences/100 patients within 12 months or 44 recurrences/100 patients within 24 months were still observed after receiving the treatment with belimumab; the two randomized trials showed that rituximab is not superior to conventional treatment in non-renal SLE and lupus nephritis. In addition, due to long-term medication, patients have been in a state of partial immunodeficiency, which increases the risk of adverse events of infection.

CD19 is a membrane surface glycoprotein of immunoglobulin superfamily with a molecular weight of 95 kDa, which is widely expressed in all stages of B cell development before plasma cell differentiation. At present, CD19-targeted CAR-T cell therapy has obvious advantages over other therapies in the treatment of SLE, which is mainly manifested in its good curative effect on severely refractory SLE and its long-term remission in the absence of the drug. It has been reported that CD19-targeted CAR-T therapy has been applied to a female patient with severe SLE, and the patient's condition has been significantly relieved without obvious side effects. Another literature reported 5 patients with refractory systemic lupus erythematosus whose condition was significantly improved after CD19CAR-T treatment, and during the 17-month follow-up, none of them had recurrences, all of them achieved remission in the absence of the drug, their quality of life was greatly improved.

At present, CAR-T therapies for SLE indications in the market are all in the development stage, and the products approved by IND are all targeting the CD19 target, such as KYV-101 of Kyverna Therapeutics and CABA-201 of Cabaletta Bio, which have obtained clinical implicit approval from FDA for treating lupus nephritis (LN) and active lupus nephritis (active LN) or active SLE without renal involvement, while the investigational new drug application for the treatment of moderate to severe refractory systemic lupus erythematosus by the injection of relmacabtagene autoleucel of JW Therapeutics has obtained implicit approval from the National Medical Products Administration. See Table 2 for details.

TABLE 2
Publicly approved CAR-T products for IND for SLE-related indications

| | | | | | Development& | IND | |
|---|---|---|---|---|---|---|---|
| | Product | | | | research | approval | |
| No. | name | Company | Target | Indications | phase | time | Country |
| 1 | Relmacabta | JW | CD19 | Moderate | 1/2 phase | 10 Apr. 2023 | China |
| | gene | Therapeutics | | to severe | | | |
| | autoleucel | | | refractory | | | |
| | injection | | | systemic | | | |
| | | | | lupus | | | |
| | | | | erythematosus | | | |
| 2 | KYV-101 | Kyverna | CD19 | lupus nephritis | 1 phase | 11 Nov. 2022 | U.S. |
| | | Therapeutics | | (LN) | | | |
| 3 | CABA-201 | Cabaletta | CD19 | Active lupus | 1/2 phase | 31 Mar. 2023 | U.S. |
| | | Bio, Inc | | nephritis | | | |
| | | | | or active | | | |
| | | | | systemic lupus | | | |
| | | | | erythematosus | | | |
| | | | | without | | | |
| | | | | renal | | | |
| | | | | involvement | | | |

According to the published clinical results, because the expression of CD19 on the surface of plasma cells is mostly negative or extremely low, although CAR-T therapy for CD19 can reduce the level of autoantibodies, its total IgG antibody level has no significant reduction, that is, targeting CD19-positive cells alone cannot deeply clear ASC including LLPC that leads to some refractory SLE. The treatment with CAR-T for CD19 in THE lupus mouse model also shows that although the treatment with CAR-T for CD19 can improve the symptoms of lupus, the autoantibodies can only be removed by administration at the early stage of the disease. In the late stage of disease progression, the clearance of B cells alone cannot prevent the continuous production of autoantibodies due to the accumulation of LLPC. In order to solve these problems, the inventors have provided specific chimeric antigen receptors for improved double antigens through research, so as to effectively treat and/or prevent systemic lupus erythematosus. In addition, for other B-cell autoimmune diseases involving CD19 and/or BCMA, the specific chimeric antigen receptor for double antigens disclosed herein can all have good curative effects.

### SUMMARY OF THE INVENTION

In a first aspect, disclosed is a bispecific chimeric antigen receptor (CAR) for treating and/or preventing autoimmune diseases. The first target of the CAR is CD19, and the second target is BCMA.

B cell maturation antigen (BCMA) is a type III transmembrane protein which comprises no signal peptide and comprises an extracellular region rich in cysteine, and plays a key regulatory role in the process of B cell expansion, survival, maturation and differentiation into plasma cells. BCMA is an important surface receptor for plasma cells, and its knockout will lead to a significant decrease in plasma cells dominated by LLPC in mice, and BCMA can promote the long-term survival of LLPC in bone marrow. Data show that the expression proportion and abundance of membrane-bound BCMA in plasma cells of SLE patients are significantly higher than those of healthy people. However, targeting BCMA alone cannot effectively clear a large number of pathogenic B cells (CD19 positive to strong positive, BCMA extremely weak positive or negative) and plasmablasts (CD19 positive to weak positive, BCMA weak positive to positive) that are activated in a specific SLE onset stage. During the treatment of a patient with diffuse large B-cell lymphoma (DLBCL) accompanied with SLE by using CAR-T therapy targeting CD19 and BCMA, it was found that targeting two antigens at the same time can not only reduce ANA to an undetectable level, but also significantly reduce the total IgG antibody level, suggesting that this dual-target combination therapy can target ASC which produces a large number of autoantibodies, and further avoid the production and recurrence of IgG autoantibodies.

According to the disclosure, it is found that targeting the combination of CD19 and BCMA can completely block the way of producing autoantibodies, and completely cover B cells, plasmablasts and plasma cells in multiple differentiation stages that produce autoantibodies, so the dual-target CAR-T therapy designed based on this concept can be used for autoimmune diseases mediated by B cells, especially for refractory SLE, and is expected to obtain faster remission and deeper and lasting curative effect.

In some embodiments, the bispecific CAR can treat or prevent B cell mediated autoimmune diseases by killing or inhibiting CD19-positive B cells, plasmablasts and BCMA-positive plasma cells. The above drugs and preparations have the ability to kill CD19-positive B cells, plasmablasts and BCMA-positive plasma cells at the same time. The combination of targets CD19 and BCMA can not only have a deeper curative effect on B cell mediated autoimmune diseases, but also have the potential to reduce the possibility of recurrence of such diseases. Its mechanism and principle are shown in FIG. 2.

In an embodiment, the bispecific CAR may be combined with one or more agents. The agent may be one that increases the efficacy of cells having CAR nucleic acids or polypeptides. The Agent may be for improving one or more side effects associated with the administration of cells comprising CAR nucleic acids or CAR polypeptide. The agent may be an additional agent for treating diseases related to BCMA and CD19. The agent may be a second therapy selected from surgery, chemotherapy, radiotherapy, immunotherapy, gene therapy, DNA therapy, RNA therapy, nanotherapy, virus therapy, adjuvant therapy and any combination thereof.

The drug can kill or inhibit CD19-positive B cells, plasmablasts and BCMA-positive plasma cells to treat autoimmune diseases.

The autoimmune disease is an autoimmune disease mediated by B cells. The autoimmune diseases mediated by B cells include systemic lupus erythematosus (SLE), glomerulonephritis includes autoimmune chronic kidney disease, lupus nephritis, acute glomerulonephritis, immune nephritis, membranous nephropathy, IgA nephropathy, Anti-Neutrophil Cytoplasmic Antibody (ANCA) Associated Vasculitis (AAV); scleroderma or systemic sclerosis (SSc); myositis or idiopathic inflammatory myositis including dermatomyositis, polymyositis, immune-mediated necrotizing myopathy (IMNM), antisynthetase syndrome, inclusion body myositis, and overlap myositis; multiple sclerosis (MS); inflammatory bowel disease (IBD); rheumatoid arthritis (RA); Sjogren's syndrome (SS); autoimmune hemolytic anemia; neuromyelitis optica (NMO); neuromyelitis optica spectrum disease (NMOSD); idiopathic thrombocytopenic purpura (ITP); systemic autoimmune small vessel vasculitis syndrome or polyangiitis related to antineutrophil cytoplasmic antibody; Wegener's granulomatosis (GPA), eosinophilic granulomatosis with polyangiitis (EGPA, Churg-Strauss syndrome); pemphigus vulgaris; autoimmune encephalitis; pemphigus vulgaris; myasthenia gravis; antiphospholipid syndrome; Chagas' disease; Graves' disease; polyarteritis nodosa; pulmonary hemorrhage-nephritis syndrome; Kawasaki disease, amyloidosis; monoclonal immunoglobulin of undetermined significance, POEMS syndrome; Crohn's disease; ulcerative colitis; adult onset Still's disease; and chronic progressive cortical demyelinating encephalopathy (CIDP).

In an embodiment, the autoimmune disease mediated by B cells is systemic lupus erythematosus, and in some of the above embodiments, the systemic lupus erythematosus includes moderate to severe refractory systemic lupus erythematosus, lupus nephritis, active lupus nephritis and active systemic lupus erythematosus without renal involvement.

In an embodiment, the autoimmune disease mediated by B cells is myositis.

In an embodiment, the autoimmune disease mediated by B cells is glomerulonephritis, and in some embodiments the glomerulonephritis is at least one of autoimmune chronic kidney disease, lupus nephritis, acute glomerulonephritis, immune nephritis, membranous nephropathy, and IgA nephropathy.

In an embodiment, the autoimmune disease mediated by B cells is multiple sclerosis.

In an embodiment, the autoimmune disease mediated by B cells is scleroderma or systemic sclerosis (SSc).

In an embodiment, the autoimmune disease mediated by B cells is myasthenia gravis.

The second aspect disclosed herein is the structure of the chimeric antigen receptor. The first antibody targeting BCMA or an antigen-binding fragment thereof or the second antibody targeting CD19 or an antigen-binding fragment thereof is independently selected from camel Ig, IgNAR, Fab fragment, Fab′ fragment, F(ab′)z fragment, F(ab′)3 fragment, Fv, single-chain antibody such as scFv, di-scFv, and (scFv)z, micro-antibody, bifunctional antibody, trifunctional antibody, tetrafunctional antibody, disulfide bond-stabilized Fv protein (“dsFv”) and single-domain antibody (sdAb, nano-antibody), chimeric antibody, humanized antibody, single-domain antibody, bispecific antibody or multispecific antibody, binding ligand or protein domain. The antigen-binding fragment targeting BCMA or the antigen-binding fragment targeting CD19 is scFv.

In an embodiment, the bispecific CAR contains an antibody or antigen-binding fragment thereof targeting BCMA that includes a heavy chain variable region (VH, BCMA) having a complementarity determining region 1 (HCDR1) comprising the amino acid sequence of SEQ ID NO: 5, a HCDR2 amino acid sequence of SEQ ID NO: 6, and a HCDR3 amino acid sequence of SEQ ID NO: 7; and a light chain variable region (VL, BCMA) having a complementarity determining region 1 (LCDR1) amino acid sequence of SEQ ID NO: 8, a LCDR2 amino acid sequence of SEQ ID NO: 9, and a LCDR3 amino acid sequence of SEQ ID NO: 10.

In an embodiment, the bispecific CAR contains an antibody or antigen-binding fragment thereof targeting CD19 that includes a heavy chain variable region (VH, CD19) having a complementarity determining region 1 (HCDR1) comprising the amino acid sequence of SEQ ID NO: 11, a HCDR2 amino acid sequence of SEQ ID NO: 12, and a HCDR3 amino acid sequence of SEQ ID NO: 13; and a light chain variable region (VL, CD19) having a complementarity determining region 1 (LCDR1) amino acid sequence of SEQ ID NO: 14, a LCDR2 amino acid sequence of SEQ ID NO: 15, and a LCDR3 amino acid sequence of SEQ ID NO: 16.

In an embodiment, the bispecific CAR includes the antibody or antigen-binding fragment thereof targeting BCMA with the above-mentioned CDRs (SEQ ID NOs 5-10) and the antibody or antigen-binding fragment thereof targeting CD19 with the above-mentioned CDRs (SEQ ID NOs 11-16).

In an embodiment, the bispecific CAR containing the VH, BCMA includes an amino acid sequence having at least 95% identity to SEQ ID NO: 1. In an embodiment the VL, BCMA includes an amino acid sequence having at least 95% identity to SEQ ID NO: 2.

In an embodiment, the bispecific CAR containing the VH, CD19 includes an amino acid sequence having at least 95% identity to SEQ ID NO: 3. In an embodiment, the VL, CD19 includes an amino acid sequence having at least 95% identity to SEQ ID NO: 4.

In some embodiments the BCMA antigen binding fragment includes both SEQ ID NOs 1 and 2 or a sequence having at least 95 percent amino acid identity thereto; and/or the CD19 antigen binding fragment includes both SEQ ID NOs 3 and 4 or a sequence having at least 95 percent amino acid identity thereto.

In an embodiment, the bispecific CAR may have a structure selected from any one of the following formulas:
1a) L-VL, CD19-VH, BCMA-VL, BCMA-VH, CD19-H-TM-C-CD3ζ;
1b) L-VL, CD19-VL, BCMA-VH, BCMA-VH, CD19-H-TM-C-CD3ζ;
2a) L-VH, CD19-VL, BCMA-VH, BCMA-VL, CD19-H-TM-C-CD3ζ;
2b) L-VH, CD19-VH, BCMA-VL, BCMA-VL, CD19-H-TM-C-CD3ζ;
3a) L-VL, CD19-VH, CD19-H-TM-C-CD3ζ-2A peptide-L-VH, BCMA-VL, BCMA-H-TM′-C-CD3ζ;
3b) L-VH, CD19-VL, CD19-H-TM-C-CD3ζ-2A peptide-L-VH, BCMA-VL, BCMA-H-TM′-C-CD3ζ;
3c) L-VL, CD19-VH, CD19-H-TM-C-CD3ζ-2A peptide-L-VL, BCMA-VH, BCMA-H-TM′-C-CD3ζ;
3d) L-VH, CD19-VL, CD19-H-TM-C-CD3ζ-2A peptide-L-VL, BCMA-VH, BCMA-H-TM′-C-CD3ζ;
4a) L-VH, BCMA-VL, BCMA-H-TM-C-CD3ζ-2A peptide-L-VL, CD19-VH, CD19-H-TM′-C-CD3ζ;
4b) L-VL, BCMA-VH, BCMA-H-TM-C-CD3ζ-2A peptide-L-VL, CD19-VH, CD19-H-TM′-C-CD3ζ;
4c) L-VH, BCMA-VL, BCMA-H-TM-C-CD3ζ-2A peptide-L-VH, CD19-VL, CD19-H-TM′-C-CD3ζ; or
4d) L-VL, BCMA-VH, BCMA-H-TM-C-CD3ζ-2A peptide-L-VH, CD19-VL, CD19-H-TM′-C-CD3ζ.

In an embodiment, the bispecific CAR has the formula of one of 1a), 2a), 3a), or 4a).

In the above formulas 1a), 1b), 2a), and 2b) comprises a loop structure formed by the antibody or antigen binding fragments thereof targeting BCMA and CD19. For example, in formula 1a), VL, CD19-VH, BCMA-VL, BCMA-VH, CD19 constitutes a Loop structure; in formula 2a), VH, CD19-VL, BCMA-VH, BCMA-VL, CD19 constitutes a Loop structure.

In the above formulas, “−” is independently a linker peptide or peptide bond; L may be absent or present as a signal peptide sequence. L can include a domain from the following protein-derived signal peptides: CD8, CD28, GM-CSF, CD4, CD137 or a combination thereof. L may be a CD8-derived signal peptide. The signal peptide sequence may be an amino acid sequence as set forth in SEQ ID NO: 25: MALPVTALLLPLALLLHAARP;

VH, BCMA is an anti-BCMA antibody heavy chain variable region. The sequence of the VH, BCMA may include the CDRs set forth in SEQ ID NOs: 5 to 7. The sequence of the VH, BCMA antibody heavy chain variable region may be as set forth in SEQ ID NO: 1.

VL, BCMA is an anti-BCMA antibody light chain variable region. The sequence of VL, BCMA may include the CDRs set forth in SEQ ID NOs: 8 to 10. The sequence of the VL, BCMA antibody light chain variable region may be as set forth in SEQ ID NO: 2.

VH, CD19 is an anti-CD19 antibody heavy chain variable region. The sequence of VH, CD19 may include the CDRs set forth in SEQ ID NOs: 11 to 13. The sequence of the VH, CD19 antibody heavy chain variable region may be as set forth in SEQ ID NO: 3.

VL, CD19 is an anti-CD19 antibody light chain variable region. The sequence of the VL, CD19 may include the CDRs set forth in SEQ ID NOs: 14 to 16. The sequence of the VL, CD19 antibody light chain variable region is as set forth in SEQ ID NO: 4.

H is a hinge region;
TM is the first transmembrane domain;
TM′ is the second transmembrane domain;
2A peptide is selected from P2A, T2A, E2A or F2A;
C is a costimulatory signal molecule;
CD3ζ is a cytoplasmic signal transduction sequence derived from CD3ζ.

VH, BCMA is an anti-BCMA antibody heavy chain variable region, and VL, BCMA is an anti-BCMA antibody light chain variable region; the sequence of the VH, BCMA antibody heavy chain variable region is as set forth in SEQ ID NO: 1:

(SEQ ID NO: 1)
QVQLVQSGAEVKKPGASVKLSCKASGYTFTDYYIHWVRQAPGQGLEWIGY
INPNSGYTNYAQKFQGRATMTADKSINTAYVELSRLRSDDTAVYFCTRYM
WERVTGFFDFWGQGTMVTVSS

In the above sequence SEQ ID NO: 1, according to the Kabat numbering system, there are three complementarity determining regions (CRDs) of the heavy chain variable region, which are as follows:

(SEQ ID NO: 5)
HCDR1: DYYIH
(SEQ ID NO: 6)
HCDR2: YINPNSGYTNYAQKFQG
(SEQ ID NO: 7)
HCDR3: YMWERVTGFFDF

As another example, the VH, BCMA antibody heavy chain variable region further includes: a sequence comprising the amino acid sequence set forth in SEQ ID NO: 1 or having substitution, deletion or addition of one or more amino acids, for example, substitution, deletion or addition of one, two or three amino acids compared with it;

In some embodiments, the variant of the VH, BCMA antibody heavy chain variable region has at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which it is derived, or has substitution, deletion or addition of one or more amino acids compared with the sequence from which it is derived (for example, substitution, deletion or addition of 1, 2, 3, 4 or 5 amino acids). In an embodiment, the substitution is conservative.

The sequence of the VL, BCMA antibody light chain variable region is as set forth in SEQ ID NO: 2:

(SEQ ID NO: 2)
DIQMTQSPSSVSASVGDRVTITCLASEDISDDLAWYQQKPGKAPKVLVYT
TSSLQSGVPSRFSGSGSGTDFTLTISSLQPEDFATYFCQQTYKFPPTFGG
GTKVEIKR.

In the above sequence SEQ ID NO: 2, according to the Kabat numbering system, there are three complementarity determining regions (CRDs) of the light chain variable region, which are:

(SEQ ID NO: 8)
LCDR1: LASEDISDDLA
(SEQ ID NO: 9)
LCDR2: TTSSLQS
(SEQ ID NO: 10)
LCDR3: QQTYKFPPT

As another example, the VL, BCMA antibody light chain variable region further includes: a sequence comprising the amino acid sequence set forth in SEQ ID NO: 2 or having substitution, deletion or addition of one or more amino acids, for example, substitution, deletion or addition of one, two or three amino acids compared with it;

In some embodiments, the variant of the VL, BCMA antibody light chain variable region has at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which it is derived, or has substitution, deletion or addition of one or more amino acids compared with the sequence from which it is derived (for example, substitution, deletion or addition of 1, 2, 3, 4 or 5 amino acids). In an embodiment, the substitution is conservative.

VH, CD19 is an anti-CD19 antibody heavy chain variable region, and VL, CD19 is an anti-CD19 antibody light chain variable region; the sequence of the VH, CD19 antibody heavy chain variable region is as set forth in SEQ ID NO: 3:

EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSS;

the sequence of the VL, CD19 antibody light chain variable region is as set forth in SEQ ID NO: 4:

DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEIT.

In the above sequence SEQ ID NO: 3, according to the Kabat numbering system, there are three complementarity determining regions (CRDs) of the heavy chain variable region, which are as follows:

(SEQ ID NO: 11)
HCDR1: DYGVS
(SEQ ID NO: 12)
HCDR2: VIWGSETTYYNSALKS
(SEQ ID NO: 13)
HCDR3: HYYYGGSYAMDY

In the above sequence SEQ ID NO: 4, according to the Kabat numbering system, there are three complementarity determining regions (CRDs) of the light chain variable region, which are as follows:

(SEQ ID NO: 14)
LCDR1: RASQDISKYLN
(SEQ ID NO: 15)
LCDR2: HTSRLHS
(SEQ ID NO: 16)
LCDR3: QQGNTLPYT

As another example, the VH, CD19 further includes: a sequence comprising an amino acid sequence set forth in SEQ ID NO: 3 or having substitution, deletion or addition of one or more amino acids, for example, substitution, deletion or addition of one, two or three amino acids compared with it;

In some embodiments, the VH, CD19 variant has at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which it is derived, or has substitution, deletion or addition of one or more amino acids compared with the sequence from which it is derived (for example, substitution, deletion or addition of 1, 2, 3, 4 or 5 amino acids) In an embodiment, the substitution is conservative.

As another example, the VL, CD19 further includes: a sequence comprising an amino acid sequence set forth in SEQ ID NO: 4 or having substitution, deletion or addition of one or more amino acids, for example, substitution, deletion or addition of one, two or three amino acids compared with it;

In some embodiments, the variant of scFv2 has at least 70%, at least 80%, at least 85%, at least 90%, at least 91%, at least 92%, at least 93%, at least 94%, at least 95%, at least 96%, at least 97%, at least 98%, at least 99%, at least 99% or 100% sequence identity compared with the sequence from which it is derived, or has substitution, deletion or addition of one or more amino acids compared with the sequence from which it is derived (for example, substitution, deletion or addition of 1, 2, 3, 4 or 5 amino acids). In an embodiment, the substitution is conservative.

H is a hinge region; H can include a domain from the following proteins: CD8, CD28, CD137 or a combination thereof; H can be selected from hinge regions derived from CD8; the H can include or consist of an amino acid sequence set forth in SEQ ID NOs: 17-19:

(SEQ ID NO: 17)
TTTPAPRPPTPAPTIASQPLSLRPEACRPAAGGAVHTRGLDFACD;
or
(SEQ ID NO: 18)
KPTTTPAPRPPTPAPTIASQPLSLRPEASRPAAGGAVHTRGLDFASDKP;
or
(SEQ ID NO: 19)
SGTTTPAPRPPTPAPTIASQPLSLRPEACRPAAGGAVHTRGLDFACD.

TM or TM′ is a (first or second, respectively) transmembrane domain; in an example, TM or TM′ can be a transmembrane region of a protein domain of CD28, CD3 epsilon, CD45, CD4, CD5, CD8, CD9, CD16, CD22, CD33, CD37, CD64, CD80, CD86, CD134, CD137, CD154 or a combination thereof;

The TM or TM′ can be a transmembrane region derived from CD8, and the transmembrane region derived from CD8 has an amino acid sequence as set forth in SEQ ID NO: 20:

(SEQ ID NO: 20)
IYIWAPLAGTCGVLLLSLVITLYC

C can be a costimulatory signal molecule; C can be a costimulatory signal molecule of a protein domain of OX40, CD2, CD7, CD27, CD28, CD30, CD40, CD70, CD134, 4-1BB (CD137), PD1, Dap10, CDS, ICAM-1, LFA-1 (CD11a/CD18), ICOS (CD278), NKG2D, GITR, TLR2 or a combination thereof;

C can be a costimulatory signal molecule derived from 4-1BB. C can be derived from 4-1BB having an amino acid sequence as set forth in SEQ ID NO: 21:

KRGRKKLLYIFKQPFMRPVQTTQEEDGCSCRFPEEEEGGCEL.

CD3ζ is a cytoplasmic signal transduction sequence derived from CD3ζ. In an embodiment, the CD3ζ signaling region has an amino acid sequence as set forth in SEQ ID NO: 22:

RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

In another example, the CAR (for example, at the C-terminus or N-terminus) further comprises a cell suicide element; wherein the cell suicide element can be linked to the CAR or the L or CD3ζ of the bispecific CAR through T2A.

In another example the linker is absent or is an amino acid sequence as set forth in SEQ ID NO: 23: GGGGS. In some embodiments, the linker sequence in each instance may independently be absent or correspond to SEQ ID NO: 23 or a repeat of SEQ ID NO: 23. In an embodiment, each instance of a linker is independently absent or repeated from 1 to 10 times, from 1 to 5 times, from 1 to 3 times, or one time.

In another embodiment, in each instance, a linker sequence between each of the variable chains of BCMA and/or CD19 may independently be absent, be SEQ ID NO: 23 or some repeat thereof, and/or be an amino acid sequence as set forth in SEQ ID NO: 24:

GSTSGSGKPGSGEGSTKG.

In an embodiment, the bispecific CAR may include the amino acid sequences of SEQ ID NOs: 5 to 16. In an embodiment, the bispecific CAR may include the amino acid sequences of SEQ ID NOs: 5 to 16, one of SEQ ID NOs: 17 to 19, and SEQ ID NOs: 20 to 22. In another embodiment, the bispecific CAR further includes at least one instance of each of SEQ ID NOs: 23 to 25. In a specific embodiment, the bispecific CAR includes SEQ ID NOs: 5 to 16, 19 to 22, and 25. In a specific embodiment, the bispecific CAR includes SEQ ID NOs: 5 to 16, 19 to 22, and 25 and at least one instance of SEQ ID NOs: 23 and 24. In each of these embodiments, the order of the heavy and light chain variable regions for BCMA and CD19 may be adjusted. Example structural order of the BCMA and CD19 heavy chains includes, but is not limited to: V_{H, BCMA}-V_{L, BCMA}-V_{H, CD19}-V_{L, CD19}; V_{L, BCMA}-V_{H, BCMA}-V_{H, CD19}-V_{L, CD19}; V_{H, BCMA}-V_{L, BCMA}-V_{L, CD19}-V_{H, CD19}; V_{L, BCMA}-V_{H, BCMA}-V_{L, CD19}-V_{H, CD19}; V_{H, CD19}-V_{L, CD19}-V_{H, BCMA}-V_{L, BCMA}-V_{L, CD19}-V_{H, CD19}-V_{H, BCMA}-V_{L, BCMA}; V_{H, CD19}-V_{L, CD19}-V_{L, BCMA}-V_{H, BCMA}; V_{L, CD19}-V_{H, CD19}-V_{L, BCMA}-V_{H, BCMA}; V_{H, CD19}-V_{L, BCMA}-V_{H, BCMA}-V_{L, CD19}; V_{H, CD19}-VH, BCMA-V_{L, BCMA}-V_{L, CD19}; V_{L, CD19}-V_{L, BCMA}-V_{H, BCMA}-V_{H, CD19}; V_{L, CD19}-V_{H, BCMA}-VL, BCMA-V_{H, CD19}; V_{H, BCMA}-V_{L, CD19}-V_{H, CD19}-V_{L, BCMA}-V_{H, BCMA}-V_{H, CD19}-V_{L, CD19}-V_{L, BCMA}-V_{L, BCMA}-V_{L, BCMA}-V_{H, CD19}-V_{H, CD19}; or V_{L, BCMA}-V_{H, BCMA}-V_{L, CD19}-V_{H, CD19}.

In an embodiment, the bispecific CAR may include the amino acid sequences of SEQ ID NOs: 1 to 4. In an embodiment, the bispecific CAR may include the amino acid sequences of SEQ ID NOs: 1 to 4, one of SEQ ID NOs: 17 to 19, and SEQ ID NOs: 20 to 22. In an embodiment, the bispecific CAR further includes at least one instance of each of SEQ ID NOs: 23 to 25. In a specific embodiment, the bispecific CAR includes SEQ ID NOs: 1 to 4, 19 to 22, and 25. In a very specific embodiment, the bispecific CAR includes SEQ ID NOs: 1 to 4, 19 to 22, and 25 and at least one instance of SEQ ID NOs: 23 and 24.

In an embodiment, the bispecific CAR includes: H having the amino acid sequence of SEQ ID NO: 19; L having the amino acid sequence of SEQ ID NO: 25; V_{H, BCMA} having the amino acid sequence of SEQ ID NO: 1; VL, BCMA having the amino acid sequence of SEQ ID NO: 2; VH, CD19 having the amino acid sequence of SEQ ID NO: 3; VL, CD19 having the amino acid sequence of SEQ ID NO: 4; TM having the amino acid sequence of SEQ ID NO: 20; C having the amino acid sequence of SEQ ID NO: 21; and CD3ζ having the amino acid sequence of SEQ ID NO: 22. The structure of the bispecific CAR may correspond to formula 1a).

In an embodiment, the bispecific CAR has the amino acid sequence as set forth in SEQ ID NO: 26:

MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSQVQLVQSGAEVKKPGAS
VKLSCKASGYTFTDYYIHWVRQAPGQGLEWIGYINPNSGYTNYAQKFQGR
ATMTADKSINTAYVELSRLRSDDTAVYFCTRYMWERVTGFFDFWGQGTMV
TVSSGSTSGSGKPGSGEGSTKGDIQMTQSPSSVSASVGDRVTITCLASED
ISDDLAWYQQKPGKAPKVLVYTTSSLQSGVPSRFSGSGSGTDFTLTISSL
QPEDFATYFCQQTYKFPPTFGGGTKVEIKRGGGGSEVKLQESGPGLVAPS
QSLSVTCTVSGVSLPDYGVSWIRQPPRKGLEWLGVIWGSETTYYNSALKS
RLTIIKDNSKSQVFLKMNSLQTDDTAIYYCAKHYYYGGSYAMDYWGQGTS
VTVSSSGTTTPAPRPPTPAPTIASQPLSLRPEACRPAAGGAVHTRGLDFA
CDIYIWAPLAGTCGVLLLSLVITLYCKRGRKKLLYIFKQPFMRPVQTTQE
EDGCSCRFPEEEEGGCELRVKFSRSADAPAYKQGQNQLYNELNLGRREEY
DVLDKRRGRDPEMGGKPRRKNPQEGLYNELQKDKMAEAYSEIGMKGERRR
GKGHDGLYQGLSTATKDTYDALHMQALPPR.

In an embodiment, a bispecific CAR as described above is administered to a subject in an amount of any one of 0.5±20%×10\hat{ }5 cells/kg, 1.0±20%×10\hat{ }5 cells/kg, 2.0±20%×10\hat{ }5 cells/kg, or 3.0±20%×10\hat{ }5 cells/kg. In an embodiment, the bispecific CAR is administered to a subject in an amount of 0.5×10\hat{ }5 cells/kg, 1.0×10\hat{ }5 cells/kg, 2.0×10\hat{ }5 cells/kg, or 3.0×10\hat{ }5 cells/kg.

In an embodiment, a pharmaceutical composition for treating and/or preventing an autoimmune disease is provided that includes the bispecific CAR as described herein.

In an embodiment, a method for treating and/or preventing an autoimmune disease is disclosed that includes administering to a subject in need thereof treatment of the bispecific CAR as described herein.

In an embodiment, use of the bispecific CAR as described herein for treating of and/or preventing an autoimmune disease is disclosed herein. The use may include preparation of a medicament, drug, or preparation, for the treatment and/or prevention of the autoimmune disease.

The third aspect disclosed herein is a BCMA-CD19 CAR-T cells in the treatment and/or prevention of a B cell mediated autoimmune disease such as SLE, and the BCMA-CD19 CAR-T cells are engineered autologous T cells that express the above CAR on the cell membrane. Patients' T cells were collected and transduced with CAR gene by using lentivirus (four-plasmid system package), so that T cells could specifically recognize BCMA and CD19 antigens expressed on the surface of target cells.

In an embodiment, use of BCMA-CD19 CAR-T cells in preparing drugs for treating and/or preventing the B cell mediated autoimmune disease is provided, and the BCMA-CD19 CAR-T cells can be engineered T cells expressing the above chimeric antigen receptor on the cell membrane, such as engineered autologous T cells, or engineered T cells encoding the nucleotide sequence of the above chimeric antigen receptor, such as engineered autologous T cells.

The fourth aspect discloses a nucleic acid molecule encoding the bispecific CAR disclosed herein for the treatment of an autoimmune disease. In an embodiment, a recombinant vector is disclosed for treating and/or preventing autoimmune disease. The vector may include the bispecific CAR as disclosed herein or the nucleic acid molecule.

In an embodiment, disclosed is use of a nucleic acid molecule in the preparation of a drug for treating a B cell mediated autoimmune disease such as systemic lupus erythematosus, and the nucleic acid molecule comprises a nucleotide sequence encoding the above chimeric antigen receptor.

The fifth aspect disclosed herein is use of a vector in the treatment of the B cell mediated autoimmune disease, wherein the vector is selected from DNA vectors, RNA vectors, plasmids, liposomes, microparticles, transposon vectors, CRISPR/Cas9 vectors or viral vectors; in an embodiment, the vector is a lentiviral vector.

The sixth aspect disclosed herein is use of BCMA-CD19 CAR-T cells in preparing drugs for treating and/or preventing a B cell mediated autoimmune disease such as systemic lupus erythematosus, and the BCMA-CD19 CAR-T cells are engineered autologous T cells expressing the above chimeric antigen receptor on the cell membrane.

The seventh aspect disclosed herein is a drug or preparation comprising the chimeric antigen receptor, the nucleic acid molecule, the vector, or the BCMA-CD19 CAR-T cell, and optionally, a pharmaceutically acceptable carrier, adjuvant and/or excipient; the drug or preparation can be used for treating and/or preventing a B cell mediated autoimmune disease such as systemic lupus erythematosus.

Compared with the prior art, the subject matter disclosed herein has at least the following obvious advantages and effects:
BCMA-CD19 CAR-T has the potential to clear B cells (including B precursor cells) and plasma cells at the same time, and can clear the main source of autoantibodies and realize local immune reset, so that patients with a B cell mediated autoimmune disease such as refractory SLE can obtain long-term and sustained remission.

### BRIEF DESCRIPTION OF THE DRAWINGS

FIG. 1A is a schematic diagram of an example of the structure of anti-CD19/BCMA CAR;

FIG. 1B is a schematic diagram of an example of construction of anti-CD19/BCMA CAR;

FIG. 2 shows the design of BCMA-CD19 CAR and its mechanism of treating SLE or other autoimmune diseases;

FIG. 3 shows the CAR expression of BCMA CAR-T cells;

FIG. 4 shows the CAR expression of CD19 CAR-T cells;

FIG. 5 shows the CAR expression of BCMA-CD19 CAR-T cells of the present application;

FIG. 6 shows the CAR expression of the blank control not modified with specific targets;

FIG. 7A shows the in vitro differentiation of antibody secreting cells for different antigens;

FIG. 7B shows flow cytometry images of the expression levels of different cell surface antigens;

FIG. 8 show the flow cytometry indicating the number of cells after in vitro killing of NT cells;

FIG. 9 show the flow cytometry indicating the number of cells after in vitro killing of CD19 CAR-T cells;

FIG. 10 show the flow cytometry indicating the number of cells after in vitro killing of BCMA CAR-T cells;

FIG. 11 show the flow cytometry indicating the number of cells after in vitro killing of BCMA-CD19 dual CAR-T cells;

FIG. 12 shows the statistical analysis of the results of flow cytometry concerning BCMA-CD19 dual-target CAR-T simultaneously clearing CD19+ and CD19− antibody-secreting cells;

FIG. 13 shows the effect of BCMA-CD19 CAR-T on killing antibody-secreting cells compared with CD19 CAR-T, BCMA CAR-T and the blank control group;

FIG. 14 is a histogram showing the number of antibody-secreting cells after treatment with different T cells at different effector-target ratios;

FIG. 15 shows the killing effect of BCMA-CD19 CAR-T cells on primary B cells at different effector-target ratios compared with the control group (5 h);

FIG. 16 shows the flow cytometry images of BCMA-CD19 CAR-T cells killing primary B cells at different effector-target ratios (5 h);

FIG. 17 shows the IFN-γ cytokine secretion level after BCMA-CD19 CAR-T cells killed primary B cells at different effector-target ratios (5 h);

FIG. 18 shows the IL-2 cytokine secretion level after BCMA-CD19 CAR-T cells killed primary B cells at different effector-target ratios (5 h);

FIG. 19 shows the TNF-α cytokine secretion level after BCMA-CD19 CAR-T cells killed primary B cells at different effector-target ratios (5 h);

FIG. 20 shows the IL-10 cytokine secretion level after BCMA-CD19 CAR-T cells killed primary B cells at different effector-target ratios (5 h);

FIG. 21 shows the IL-4 cytokine secretion level after BCMA-CD19 CAR-T cells killed primary B cells at different effector-target ratios (5 h);

FIG. 22 shows the IL-2 cytokine secretion level after BCMA-CD19 CAR-T cells killed primary B cells at different effector-target ratios (5 h);

FIG. 23 shows the flow cytometry images of BCMA-CD19 CAR-T cells killing antibody-secreting cells at different effector-target ratios (5 h);

FIG. 24 is a histogram showing BCMA-CD19 CAR-T cells killing antibody-secreting cells (including those comprising a single target CD19 or BCMA, and those comprising double targets) at different effector-target ratios (5 h);

FIG. 25 is a histogram showing the cytokine secretion level after BCMA-CD19 CAR-T cells killed antibody-secreting cells at different effector-target ratios (5 h);

FIG. 26 shows the gating and flow cytometry images of B cells and ASC cells (including plasma cell CD138+) in peripheral blood samples of SLE patients;

FIG. 27A shows the flow cytometry images of BCMA-CD19 CAR-T cells killing B cells of SLE patients in vitro;

FIG. 27B is a histogram showing the number of B cells after in vitro killing by BCMA-CD19 CAR-T cells;

FIG. 28A shows the flow cytometry images of BCMA-CD19 CAR-T cells killing ASC cells of SLE patients in vitro;

FIG. 28B is a histogram showing the number of ASC cells after in vitro killing by BCMA-CD19 CAR-T cells;

FIG. 29A shows the flow cytometry images of BCMA-CD19 CAR-T cells killing plasma cells of SLE patients in vitro;

FIG. 29B is a histogram showing the number of plasma cells after in vitro killing by BCMA-CD19 CAR-T cells;

FIG. 30 shows a characterization of CD19 and BCMA expression on B cell subsets from peripheral blood mononuclear cells (PBMCs) from donors having one of the following diseases Sjogren's Syndrome, scleroderma or systemic sclerosis (SSc), myositis, and IgA nephropathy, and healthy donors.

FIG. 31A shows the gating strategy for an in vitro killing assay with the BCMA-CD19 CAR-T cells.

FIG. 31B shows representative flow plots of in vitro killing.

FIG. 32 shows in vitro differentiated plasmablasts and CD19+ cells from healthy donors. The naïve B-cells were then differentiated using a proprietary mixture of cytokines to drive plasmablast differentiation (BCMA+ B-cells). After 5 days of differentiation, the cells were then co-cultured with the BCMA-CD19 Bz CAR-T cells (manufactured with preclinical manufacturing process) or untransduced T cells (untargeted) at varying effector:target (E:T) ratios. A dose dependent depletion (E:T) by BCMA-CD19 CAR-T cells is observed.

FIG. 33 shows BCMA-CD19 CAR-T cells co-cultured with in vitro differentiated B cell cultures derived from healthy donors. A dose dependent induction of cytokine production associated with T cell activation from the BCMA-CD19 CAR-T cells compared to untransduced control T cells. Supernatants were collected from co-cultures of BCMA-CD19 CAR-T cells with healthy in vitro differentiated B cells, aliquoted and stored for MSD analysis. Prior to thawing the supernatants, MSD UPLEX plates were coated with antibody linker pairs for the following analytes: IFNγ, IL-2, IL-4, IL-6, IL-10 and TNFα. Samples were thawed and diluted 1:15 in diluent 57 then incubated on the coated MSD plates. Following the sample incubation the plates were incubated with the sulfo tagged secondary antibodies prior to reading the plate. Analysis was carried out using the discovery workbench software;

FIG. 34 shows in vitro differentiated plasmablasts and CD19+ cells from Sjogren's syndrome donors show a dose dependent depletion (E:T) by BCMA-CD19 CAR-T cells. Naïve B-cells from frozen Sjogren's Syndrome patients' PBMCs were isolated with Naïve B-cell isolation kit (Stemcell). After isolation, naïve B-cells were plated and differentiated utilizing a proprietary mix of cytokines to drive B-cell differentiation. After 5 days of differentiation, the differentiated B-cells were co-cultured with BCMA-CD19 CAR-T cells or untransduced T cells at 1.5:1, 0.75:1, 0.37:1, 0.18:1, and 0.093:1 effector:target ratios for 24 hours. Co-cultured cells were then stained with the following reagents: (Live/Dead stain, CD20, CD19, CD27, CD38, BCMA, CD3, CD4, CD8, CD69). Killing of target differentiated plasma blasts (live, CD3−, CD27+ CD38+) and CD19+ targets (live CD3−, CD27−, CD38+/−, CD19+CD20+) was assessed by flow cytometry. Percent depletion was calculated by 1-(% targets in experimental well/% average targets in stim only wells)*100.

FIG. 35 shows in vitro differentiated plasmablasts and CD19+ cells from scleroderma or systemic sclerosis (SSc) donors show a dose dependent depletion (E:T) by BCMA-CD19 CAR-T cells. Naïve B-cells from frozen scleroderma or systemic sclerosis (SSc) patients' PBMCs were isolated with Naïve B-cell isolation kit (Stemcell). After isolation, naïve B-cells were plated and differentiated utilizing a proprietary mix of cytokines to drive B-cell differentiation. After 5 days of differentiation, the differentiated B-cells were co-cultured with BCMA-CD19 CAR-T cells or untransduced T cells at 1.5:1, 0.75:1, 0.37:1, 0.18:1, and 0.093:1 effector:target ratios for 24 hours. Co-cultured cells were then stained with the following reagents: (Live/Dead stain, CD20, CD19, CD27, CD38, BCMA, CD3, CD4, CD8, CD69). Killing of target differentiated plasma blasts (live, CD3−, CD27+CD38+) and CD19+ targets (live CD3−, CD27−, CD38+/−, CD19+CD20+) was assessed by flow cytometry. Percent depletion was calculated by 1-(% targets in experimental well/% average targets in stim only wells)*100.

FIG. 36 shows in vitro differentiated plasmablasts and CD19+ cells from myositis donors show a dose dependent depletion (E:T) by BCMA-CD19 CAR-T cells. Naïve B-cells from a frozen myositis patient PBMCs were isolated with Naïve B-cell isolation kit (Stemcell). After isolation, naïve B-cells were plated and differentiated utilizing a proprietary mix of cytokines to drive B-cell differentiation. After 5 days of differentiation, the differentiated B-cells were co-cultured with BCMA-CD19 CAR-T cells or untransduced T cells at a 1.5:1 effector:target ratios for 24 hours. Co-cultured cells were then stained with the following reagents: (Live/Dead stain, CD20, CD19, CD27, CD38, BCMA, CD3, CD4, CD8, CD69). Killing of target differentiated plasma blasts (live, CD3−, CD27+CD38+) and CD19+ targets (live CD3−, CD27−, CD38+/−, CD19+CD20+) was assessed by flow cytometry. Percent depletion was calculated by 1-(% targets in experimental well/% average targets in stim only wells)*100.

FIG. 37 In vitro differentiated plasmablasts and CD19+ cells from IgA nephropathy donor show a dose dependent depletion (E:T) by BCMA-CD19 CAR-T cells. Naïve B-cells from frozen IgA nephropathy patient PBMCs were isolated with Naïve B-cell isolation kit (Stemcell). After isolation, naïve B-cells were plated and differentiated utilizing a proprietary mix of cytokines to drive B-cell differentiation. After 5 days of differentiation, the differentiated B-cells were co-cultured with BCMA-CD19 CAR-T cells or untransduced T cells at 1.5:1, 0.75:1 and 0.37:1 effector:target ratios for 24 hours. Co-cultured cells were then stained with the following reagents: (Live/Dead stain, CD20, CD19, CD27, CD38, BCMA, CD3, CD4, CD8, CD69). Killing of target differentiated plasma blasts (live, CD3−, CD27+ CD38+) and CD19+ targets (live CD3−, CD27−, CD38+/−, CD19+CD20+) was assessed by flow cytometry. Percent depletion was calculated by 1-(% targets in experimental well/% average targets in stim only wells)*100.

FIG. 38 shows BCMA-CD19 CAR-T cells co-cultured with in vitro differentiated B cell cultures derived from Sjogren's syndrome donors shows a dose dependent induction of cytokine production associated with T cell activation from the BCMA-CD19 CAR-T cells compared to untransduced control T cells. Supernatants were collected from co-cultures of BCMA-CD19 CAR-T cells with Sjogren's syndrome in vitro differentiated B cells and aliquoted and stored for MSD analysis. Prior to thawing the supernatants, MSD UPLEX plates were coated with antibody linker pairs for the following analytes: IFNγ, IL-2, IL-4, IL-6, IL-10 and TNFα. Samples were thawed and diluted 1:15 in diluent 57 then incubated on the coated MSD plates. Following the sample incubation the plates were incubated with the sulfo tagged secondary antibodies prior to reading the plate. Analysis was carried out using the discovery workbench software.

FIG. 39 shows BCMA-CD19 CAR-T cells co-cultured with in vitro differentiated B cell cultures derived from scleroderma or systemic sclerosis (SSc) donors shows a dose dependent induction of cytokine production associated with T cell activation from the BCMA-CD19 CAR-T cells compared to untransduced control T cells. Supernatants were collected from co-cultures of BCMA-CD19 CAR-T cells with scleroderma or systemic sclerosis in vitro differentiated B cells and aliquoted and stored for MSD analysis. Prior to thawing the supernatants, MSD UPLEX plates were coated with antibody linker pairs for the following analytes: IFNγ, IL-2, IL-4, IL-6, IL-10 and TNFα. Samples were thawed and diluted 1:15 in diluent 57 then incubated on the coated MSD plates. Following the sample incubation the plates were incubated with the sulfo tagged secondary antibodies prior to reading the plate. Analysis was carried out using the discovery workbench software.

FIG. 40 shows BCMA-CD19 CAR-T cells co-cultured with in vitro differentiated B cell cultures derived from a myositis donor shows a dose dependent induction of cytokine production associated with T cell activation from the BCMA-CD19 CAR-T cells compared to untransduced control T cells. Supernatants were collected from co-cultures of BCMA-CD19 CAR-T cells with Myositis in vitro differentiated B cells and aliquoted and stored for MSD analysis. Prior to thawing the supernatants, MSD UPLEX plates were coated with antibody linker pairs for the following analytes: IFNγ, IL-2, IL-4, IL-6, IL-10 and TNFα. Samples were thawed and diluted 1:15 in diluent 57 then incubated on the coated MSD plates. Following the sample incubation the plates were incubated with the sulfo tagged secondary antibodies prior to reading the plate. Analysis was carried out using the discovery workbench software.

FIG. 41 shows BCMA-CD19 CAR-T cells co-cultured with in vitro differentiated B cell cultures derived from the IgA nephropathy donor shows a dose dependent induction of cytokine production associated with T cell activation from the BCMA-CD19 CAR-T cells compared to untransduced control T cells. Supernatants were collected from co-cultures of BCMA-CD19 CAR-T cells with frozen IgA nephropathy in vitro differentiated B cells and aliquoted and stored for MSD analysis. Prior to thawing the supernatants, MSD UPLEX plates were coated with antibody linker pairs for the following analytes: IFNγ, IL-2, IL-4, IL-6, IL-10 and TNFα. Samples were thawed and diluted 1:15 in diluent 57 then incubated on the coated MSD plates. Following the sample incubation, the plates were incubated with the sulfo tagged secondary antibodies prior to reading the plate. Analysis was carried out using the discovery workbench software.

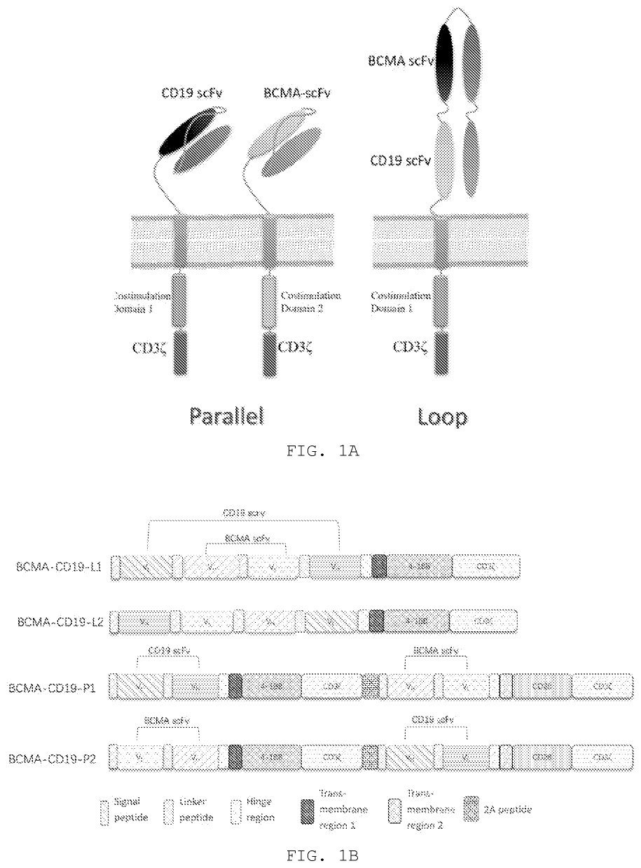

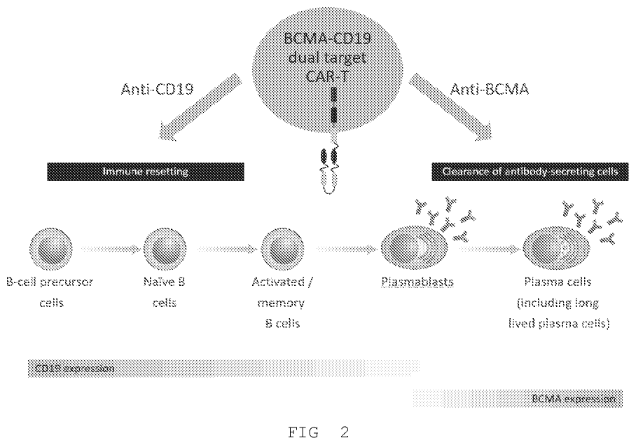

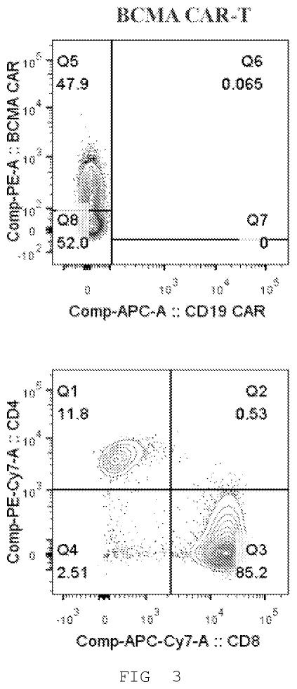

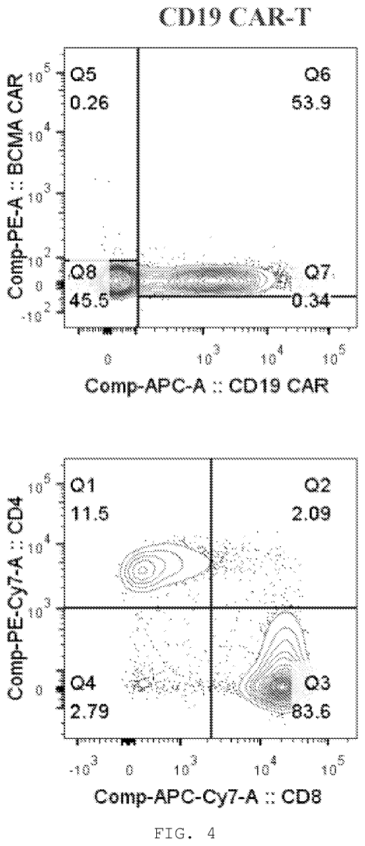

### DETAILED DESCRIPTION OF THE INVENTION

The disclosure will be further illustrated by specific examples, and it should be understood that the following examples are only for illustrating the disclosed subject matter, and are not intended to limit the content of the disclosure.

The raw materials and equipment used in the examples are well known to those skilled in the art, and are all commercially available or readily obtainable or prepared.

The term “antibody” (Ab) shall include, but not be limited to, immunoglobulins, which specifically bind to antigens and comprise at least two heavy (H) chains and two light (L) chains interconnected by disulfide bonds, or antigen-binding portions thereof. Each H chain comprises a heavy chain variable region (abbreviated as VH herein) and a heavy chain constant region. The constant region of heavy chain comprises three constant domains CH1, CH2 and CH3. Each light chain comprises a light chain variable region (abbreviated as VL herein) and a light chain constant region. The constant region of light chain comprises a constant domain CL. VH and VL regions can be further subdivided into hypervariable regions called complementarity determining regions (CDR), which are interspersed with more conservative regions called framework regions (FR). Each VH or VL comprises three CDRs and four FRs, which are arranged in the following order from the amino terminus to the carboxyl terminus: FR1, CDR1, FR2, CDR2, FR3, CDR3 and FR4. The variable regions of heavy and light chains comprise binding domains that interact with antigens.

It should be understood that the names of amino acids herein are identified by single English letters commonly used in the world, and the corresponding abbreviations in three English letters of amino acid names are as follows: Ala (A), Arg (R), Asn (N), Asp (D), Cys (C), Gln (Q), Glu (E), Gly (G), His (H), Ile (I), Leu (L), Lys (K), Met (M), Phe (F), Pro (P), Ser (S), Thr (T), Trp (W), Tyr (Y), Val (V).

### Preparation of Antibody

The antibody disclosed herein can be prepared by various methods known in the art, for example, by genetic engineering recombinant technology. For example, DNA molecules encoding the heavy chain and light chain genes of the antibody of the disclosure are obtained by chemical synthesis or PCR amplification. The obtained DNA molecule is inserted into the expression vector and then transfected into the host cells. Then, the transfected host cells are cultured under specific conditions, and the antibody of the disclosure is expressed.

The antigen-binding fragment disclosed herein can be obtained by hydrolyzing intact antibody molecules (see Morimoto et al., J. Biochem. Biophys. Methods 24: 107-117 (1992) and Brennan et al., Science 229:81 (1985)). In addition, these antigen-binding fragments can also be directly produced by recombinant host cells (summarized in Hudson, Curr. Opin. Immunol. 11: 548-557 (1999); Little et al., Immunol. Today, 21: 364-370 (2000)). For example, Fab′ fragments can be obtained directly from host cells; Fab′ fragments can be chemically coupled to form F(ab′) z fragments (Carter et al., Bio/Technology, 10: 163-167 (1992)). In addition, Fv, Fab or F(ab′)z fragments can also be directly isolated from the recombinant host cell culture. Those skilled in the art are fully aware of other techniques for preparing these antigen-binding fragments.

### Conservative Substitution

As used herein, the term “conservative substitution” means an amino acid substitution that does not adversely affect or change the expected properties of a protein/polypeptide comprising an amino acid sequence. For example, conservative substitution can be introduced by standard techniques known in the art, such as site-directed mutagenesis and PCR-mediated mutagenesis. Conservative amino acid substitution includes substitution of amino acid residues with amino acid residues with similar side chains, for example, substitution with residues that are physically or functionally similar to the corresponding amino acid residues (such as similar size, shape, charge, chemical properties, including the ability to form covalent bonds or hydrogen bonds, etc.). Families of amino acid residues with similar side chains have been defined in the art. These families include amino acids with basic side chains (such as lysine, arginine and histidine), acidic side chains (such as aspartic acid and glutamic acid), uncharged polar side chains (such as glycine, asparagine, glutamine, serine, threonine, tyrosine, cysteine and tryptophan), nonpolar side chains (such as alanine, valine, leucine, isoleucine, proline, phenylalanine and methionine), β-branched side chains (e.g. threonine, valine, isoleucine) and aromatic side chains (e.g. tyrosine, phenylalanine, tryptophan, histidine). Therefore, it is preferable to substitute the corresponding amino acid residue with another amino acid residue from the same side chain family.

### B Cell Maturation Antigen (BCMA)

BCMA (also known as TNFRF17, BCM or CD269) is a member of tumor necrosis factor receptor (TNFR) family, and is mainly expressed on terminally differentiated B cells, such as memory B cells and plasma cells. Its ligands are called a B cell activating factor of the TNF family (BAFF) and a proliferation-inducing ligand (APRIL). BCMA participates in mediating the survival of plasma cells to maintain long-term humoral immunity. The gene of BCMA is encoded on chromosome 16, resulting in a primary mRNA transcript with a length of 994 nucleotides (NCBI accession number NM_001192.2), which encodes a protein of 184 amino acids (NP_001183.2). A second antisense transcript from the BCMA locus has been described, which can play a role in regulating BCMA expression (Laabi Y. et al., Nucleic Acids Res., 1994, 22: 1147-1154). Another transcript variant with undetermined importance has been described (Smirnova A S et al., Mol Immunol., 2008, 45 (4): 1179-1183). A second isoform (also called TV4) has been identified (Uniprot identifier Q02223-2).

However, some patients will still have a relapse process after receiving CAR-T cell therapy targeting BCMA. For these relapsed patients, it is necessary to find a target different from BCMA before continuing treatment.

### CD19

As used herein, the term “CD19” refers to B lymphocyte antigen CD19, also known as B lymphocyte surface antigen B4 or T cell surface antigen Leu-12, and includes any natural CD19 of any vertebrate origin, including mammals, such as primates (e.g. humans), nonhuman primates (e.g. cynomolgus monkeys) and rodents (e.g. mice and rats), unless otherwise specified. The NCBI accession number of the amino acid sequence of human CD19 is NP_001171569. The term covers “full-length”, unprocessed human CD19 and any form of human CD19 derived from processing in cells, as long as the antibodies reported herein bind to it. CD19 is a structurally unique cell surface receptor expressed on the surface of human B cells, which include but are not limited to pre-B cells, B cells in early development (i.e. immature B cells), mature B cells terminally differentiated into plasma cells and malignant B cells. CD19 is expressed by most of pre-B acute lymphoblastic leukemia (ALL), non-Hodgkin's lymphoma, B-cell chronic lymphoblastic leukemia (CLL), lymphoblastic leukemia, hairy cell leukemia, common acute lymphoblastic leukemia and some Null-acute lymphoblastic leukemia. The expression of CD19 on plasma cells further suggests that CD19 may be expressed in differentiated B-cell tumors such as multiple myeloma. Therefore, CD19 antigen is the target of immunotherapy for non-Hodgkin's lymphoma, chronic lymphoblastic leukemia and/or acute lymphoblastic leukemia.

CD19 is a glycoprotein with a molecular weight of 95 kD expressed on the membrane surface of pre-B cells and mature B cells, it is closely related to the transmembrane conduction pathway of Ca++ in B cells, and can regulate the proliferation and differentiation of B cells. CD19 is mainly expressed in normal B cells and cancerous B cells, with high tissue expression specificity, and it is a good antibody or CAR-T immunotherapy target. However, in the process of immunotherapy, the loss of CD19 epitope of B cells often occurs, which causes patients to fail to respond to immunotherapy or relapse.

### Chimeric Antigen Receptor (CAR)

The chimeric antigen receptor (CAR) of the disclosure includes extracellular domains, transmembrane domains, and intracellular domains. Extracellular domains include target-specific binding elements (also called antigen binding domains). Intracellular domains include costimulatory signal transduction region and (chain portion. Costimulatory signal transduction region refers to a part of intracellular domain including costimulatory molecules. Co-stimulatory molecules are cell surface molecules needed by lymphocytes to effectively respond to antigens, not antigen receptors or their ligands.

A linker can be incorporated between the extracellular domain and transmembrane domain of CAR, or between the cytoplasmic domain and transmembrane domain of CAR. As used herein, the term “linker” generally refers to any oligopeptide or polypeptide that serves to connect the transmembrane domain to the extracellular domain or cytoplasmic domain of the polypeptide chain. The linker may comprise 0-300 amino acids, 2-100 amino acids, and 3-50 amino acids.

In an embodiment, the extracellular domain of CAR provided by the disclosure includes an antigen binding domain targeting BCMA (or BCMA and CD19). When the CAR disclosed herein is expressed in T cells, it can recognize antigens based on antigen binding specificity. When it binds to its associated antigen, it can kill CD19-positive B cells, plasmablasts and BCMA-positive plasma cells. The antigen binding domain can be fused with an intracellular domain from one or more of the costimulatory molecule and the (chain. The antigen binding domain can be fused with the intracellular domain of the combination of the 4-1BB signal transduction domain and the CD3ζ zeta signal domain.

As used herein, “antigen binding domain” and “single-chain antibody fragment” both refer to a Fab fragment, Fab′ fragment, F(ab′)₂ fragment, or single Fv fragment with antigen binding activity. The Fv antibody comprises an antibody heavy chain variable region and light chain variable region, but has no constant region, and has the smallest antibody fragment of all antigen binding sites. In general, Fv antibodies also comprise polypeptide linkers between VH and VL domains, and can form structures required for antigen binding. The antigen binding domain is usually scFv (single-chain variable fragment). The size of scFv is generally ⅙ that of a complete antibody. The single-chain antibody can be an amino acid chain sequence encoded by a nucleotide chain. As another embodiment, the antigen binding domain comprises an antibody specifically recognizing BCMA, and optionally, the antigen binding domain further comprises an antibody specifically recognizing CD19. In an embodiment the antigen binding domain is a single-chain antibody.

For hinge region and transmembrane region (transmembrane domain), CAR can be designed to include a transmembrane domain fused to the extracellular domain of CAR. In an embodiment, a transmembrane domain naturally associated with one of the domains in CAR is used. In some examples, transmembrane domains can be selected or modified by amino acid substitution to avoid binding such domains to transmembrane domains of the same or different surface membrane proteins, thus minimizing interaction with other members of the receptor complex.

The intracellular domains in the disclosed CAR include 4-1BB signal transduction domains and CD3ζ signal transduction domains.

The CAR disclosed herein also includes a cell suicide element.

### Bispecific CAR Targeting CD19 and BCMA

Bispecificity means that the same CAR can specifically bind to and immuno-recognize two different antigens, and immune response can be produced when CAR binds to any of the antigens. The bispecific CAR is a dual-target CAR.

In another example, the bispecific CAR targeting CD19 and BCMA is as described in the first aspect of the disclosure.

In an embodiment, the extracellular domain of CAR provided by the disclosure includes antigen binding domains targeting CD19 and BCMA, including anti-CD19 scFv and anti-BCMA scFv.

In another example, the disclosure provides a bispecific CAR for CD19 and BCMA antigens. The structural components of CAR targeting both CD19 and BCMA can include signal peptide, anti-CD19 scFv, anti-BCMA scFv, hinge region, transmembrane region, and intracellular T cell signal region, in which CD19scFv and BCMA scFv are connected by a short peptide segment (G4S)×N. The CAR structure targeting both CD19 and BCMA is as described in the second aspect of the disclosure.

In another example, the sequence of the BCMA scFv is optimized in the disclosure, and the BCMA scFv (S scFv) has high affinity with BCMA and good specificity, and can specifically target BCMA full-length antigen and extracellular region.

In an embodiment, (G4S)×3 is used to connect CD19scFv and BCMAscFv, at which time the activity and lethality of CAR are the best.

Compared with CAR targeting a single antigen, a bispecific CAR targeting CD19 and BCMA as disclosed herein has significantly enhanced affinity, with the activity of immune cells significantly increased, and a synergistic effect is provided, as proved in the following examples. In addition, due to the uneven expression levels of CD19 and BCMA in B cells and plasma cells, dual-target CAR-T has a wider therapeutic range. CAR-T immune cells targeting CD19 and BCMA at the same time can reduce the possibility of antigen escape caused by down-regulation or deletion of a single surface antigen. In addition, targeting the combination of CD19 and BCMA can block the pathway of producing autoantibodies more comprehensively, which covers B cells, plasmablasts and plasma cells in multiple differentiation stages that produce autoantibodies more completely. The target combination of CD19 and BCMA not only has a deeper curative effect on SLE, but also has the potential to reduce the possibility of recurrence.

In an embodiment, the bispecific CAR includes the amino acid sequence represented by SEQ ID NO: 26.

### Chimeric Antigen Receptor T Cells (CAR-T Cells)

As Used Herein, the Terms “CAR-T Cell”, “CAR-T Cell” and “CAR-T cell of the disclosure” include BCMA-CD19 CAR-T cells included in the third aspect of the disclosure.

### Suicide Gene Switch

In order to further control the non-tumor targeting and cytokine release syndrome of CAR-T cells, CAR cells in the disclosure are all equipped with suicide gene switches, which, under the action of exogenous drugs, can effectively clear CAR-T cells in vivo and block unknown or uncontrollable long-term toxicity to ensure the safety of patients.

The suicide switch used in the disclosure can be the herpes simplex virus thymidine kinase (HSV-TK), inducible caspase9 (iCasp9), CD20, mutated human thymidylate kinase (mTMPK) and the like. Comparatively speaking, HSV-TK, iCasp9 and CD20 have the same clearing ability on CAR-T cells, but iCasp9 and CD20 have a faster clearing rate, while HSV-TK has a slower clearing rate.

The iCasp9 suicide switch comprises FKBP12-F36V domain, which can be linked to cysteine aspartic protease 9 through a flexible linker, and the latter comprises no recruitment domain. FKBP12-F36V comprises FKBP domain, and phenylalanine replaces valine at the 36th amino acid residue. It has high selectivity and sub-nano molar affinity, and can form ligands in combination with dimerization, such as other inert small molecules AP1903. When small molecules are added, they can promote its dimerization, thus inducing cell apoptosis, but it is ineffective for normal cells without suicide switches.

Inducing safety switch caspase9 (iCasp9) uses human caspase9 to fuse with FK506 binding protein (FKBP), so that it can be induced to form dimer by a chemical inducer (AP1903/Rimiducid, Bellicum Pharmaceutical), leading to apoptosis of cells expressing the fusion protein.

Although CD19 and BCMA are highly expressed in tumor cells, they are also expressed in normal B cells, and the engineered immune cells of the disclosure will attack normal B cells in vivo.

How to control the safety of CAR-T cells has always been an urgent problem. Adding a safety switch to CAR-T cells is the safest way to stop the activity of CAR-T cells. The inducible iCasp9 safety switch controls the clearance of CAR-T cells after the CAR-T cells become severely toxic (CRS/neurotoxicity) or after the patients reach long-term continuous remission.

### Vector

The nucleic acid sequence encoding the desired molecule can be obtained by recombination methods known in the art, such as, for example, screening a library from cells expressing the gene, obtaining the gene from a vector known to include the gene, or directly isolating the gene from cells and tissues comprising the gene by using standard techniques. Optionally, the gene of interest can be synthetically produced.

The disclosure further provides a vector into which the expression cassette of the disclosure is inserted. Vectors derived from retroviruses such as lentiviruses are suitable tools to achieve long-term gene transfer, because they allow long-term and stable integration of transgenes and their proliferation in daughter cells. Lentiviral vectors have advantages over vectors derived from oncogenic retroviruses such as murine leucovirus, because they can transduce nonproliferative cells such as hepatocytes. They also have the advantage of low immunogenicity.

Briefly, the expression cassette or nucleic acid sequence of the disclosure is usually operably linked to a promoter and incorporated into an expression vector. The vector is suitable for replicating and integrating eukaryotic cells. Typical cloning vectors comprise transcription and translation terminators, initial sequences and promoters that can be used to regulate the expression of desired nucleic acid sequences.

The expression construct of the disclosure can also be used for nucleic acid immunization and gene therapy by using standard gene delivery protocols. Methods of gene delivery are known in the art. See, for example, U.S. Pat. Nos. 5,399,346, 5,580,859 and 5,589,466, which are incorporated herein by reference in their entirety. In another embodiment, the disclosure provides a gene therapy vector.

The nucleic acid can be cloned into many types of vectors. For example, the nucleic acid can be cloned into such vectors, including but not limited to plasmids, phagemids, phage derivatives, animal viruses and cosmids. Specific vectors of interest include expression vectors, replication vectors, probe-generating vectors and sequencing vectors.

Further, the expression vector can be provided to the cell in the form of a viral vector. viral vector technology is well known in the art and described in, for example, Sambrook et al. (2001, Molecular Cloning: A Laboratory Manual, Cold Spring Harbor Laboratory, New York) and other manuals of virology and molecular biology. Viruses that can be used as vectors include but are not limited to retroviruses, adenoviruses, adeno-associated viruses, herpes viruses and lentiviruses. Generally, suitable vectors comprise an origin of replication, a promoter sequence, a convenient restriction enzyme site and one or more selectable markers (e.g., WO01/96584; WO01/29058; and U.S. Pat. No. 6,326,193).

Many virus-based systems have been developed for transferring genes into mammalian cells. For example, retroviruses provide a convenient platform for gene delivery systems. The selected gene can be inserted into a vector and packaged into retrovirus particles using techniques known in the art. The recombinant virus can then be isolated and delivered to the target cells in vivo or in vitro. Many retrovirus systems are known in the art. In some embodiments, adenoviral vectors are used. Many adenoviral vectors are known in the art. In an embodiment, lentiviral vectors are used.

Additional promoter elements, such as enhancers, can adjust the frequency of transcription initiation. Generally, these are located in the 30-110 bp region upstream of the initiation site, although it has been recently shown that many promoters also comprise functional elements downstream of the initiation site. The spacing between promoter elements is often flexible, so as to maintain the promoter function when one element is inverted or moved relative to another. In the thymidine kinase (tk) promoter, the spacing between promoter elements can be increased by 50 bp before the activity begins to decline. Depending on the promoter, it is shown that individual elements can work cooperatively or independently to start transcription.

An example of a suitable promoter is the immediate early cytomegalovirus (CMV) promoter sequence. The promoter sequence is a strongly constitutive promoter sequence capable of driving high-level expression of any polynucleotide sequence operably linked thereto. Another example of a suitable promoter is elongation factor-1α(EF-1α). However, other constitutive promoter sequences can also be used, including, but not limited to, simian virus 40 (SV40) early promoter, mouse breast cancer virus (MMTV), human immunodeficiency virus (HIV) long terminal repeat (LTR) promoter, MoMuLV promoter, avian leucovirus promoter, Epstein-Barr virus immediate early promoter, Rous sarcoma virus promoter, and human gene promoters, including but not limited to actin promoter, myosin promoter, heme promoter and creatine kinase promoter. Further, the disclosure should not be limited to the application of constitutive promoters. Inducible promoters are also considered as part of the invention. The use of an inducible promoter provides a molecular switch that can turn on the expression of a polynucleotide sequence operably linked to the inducible promoter when such expression is desired, or turn off the expression when the expression is undesirable. Examples of inducible promoters include, but are not limited to, metallothionein promoters, glucocorticoid promoters, progesterone promoters and tetracycline promoters.

In order to evaluate the expression of CAR polypeptide or a portion thereof, the expression vector introduced into cells can also comprise either or both of selectable marker genes or reporter genes, so as to identify and select expression cells from the cell population seeking to be transfected or infected by viral vectors. In other aspects, selectable markers can be carried on a single piece of DNA and used in co-transfection procedures. Both the selectable marker and the reporter gene can be flanked by appropriate regulatory sequences so that they can be expressed in the host cell. Useful selectable markers include, for example, antibiotics resistance genes such as neo.

Reporter genes are used to identify potential transfected cells and to evaluate the functionality of regulatory sequences. Generally, a reporter gene is a gene that does not exist in or is expressed by a recipient organism or tissue, and that encodes a polypeptide whose expression is clearly indicated by some easily detectable properties such as enzyme activity. After DNA has been introduced into the recipient cells, the expression of the reporter gene is determined at appropriate time. Suitable reporter genes may include genes encoding luciferase, beta-galactosidase, chloramphenicol acetyltransferase, secreted alkaline phosphatase or green fluorescent protein (for example, Ui-Tei et al., 2000 FEBS Letters 479:79-82). Suitable expression systems are well known and can be prepared using known techniques or obtained commercially. Generally, a construct with at least 5 flanking regions showing the highest level of reporter gene expression is identified as a promoter. Such promoter regions can be linked to reporter genes and used to evaluate the ability of reagents to regulate promoter-driven transcription.

Methods of introducing genes into cells and expressing genes into cells are known in the art. In the context of an expression vector, the vector can be easily introduced into host cells, such as mammalian, bacterial, yeast or insect cells, by any method in the art. For example, the expression vector can be transferred into the host cell by physical, chemical or biological means.

Physical methods of introducing polynucleotides into host cells include calcium phosphate precipitation, lipid transfection, particle bombardment, microinjection, electroporation and so on. Methods for producing cells comprising vectors and/or exogenous nucleic acids are well known in the art. See, for example, Sambrook et al. (2001, molecular cloning: Molecular Cloning: A Laboratory Manual, Cold Spring Harbor Laboratory, New York). Polynucleotides can be introduced into host cells using calcium phosphate transfection.

Biological methods for introducing polynucleotides of interest into host cells include the use of DNA and RNA vectors. Viral vectors, especially retroviral vectors, have become the most widely used method to insert genes into mammalian cells, such as human cells. Other viral vectors can be derived from lentivirus, poxvirus, herpes simplex virus I, adenovirus, adeno-associated virus and so on. See, for example, U.S. Pat. Nos. 5,350,674 and 5,585,362.

Chemical means of introducing polynucleotides into host cells include colloidal dispersion systems, such as macromolecular complexes, nanocapsules, microspheres and beads; and lipid-based systems including oil-in-water emulsions, micelles, mixed micelles and liposomes. Exemplary colloidal systems used as delivery vehicles in vitro and in vivo are liposomes (e.g., artificial membrous sacs).

Where a non-viral delivery system is used, an exemplary delivery vehicle is a liposome. It is considered to use lipid preparations to introduce nucleic acids into host cells (in vitro, ex vivo or in vivo). In another aspect, the nucleic acid can be associated with lipids. Nucleic acids associated with lipids can be encapsulated in the aqueous interior of liposomes, dispersed in the lipid bilayer of liposomes, attached to liposomes via linking molecules associated with both liposomes and oligonucleotides, trapped in liposomes, complexed with liposomes, dispersed in solutions comprising lipids, mixed with lipids, combined with lipids, contained in lipids as suspensions, contained in micelles or complexed with micelles, or otherwise associated with lipids. The lipid, lipid/DNA or lipid/expression vector associated with the composition is not limited to any specific structure in solution. For example, they may exist in a bilayer structure as micelles or have a “collapsed” structure. They can also be simply dispersed in solution, and may form aggregates with uneven size or shape. Lipids are fatty substances, which can be naturally occurring or synthetic lipids. For example, lipids include fat droplets, which naturally occur in cytoplasm and such compounds comprising long-chain aliphatic hydrocarbons and their derivatives such as fatty acids, alcohols, amines, aminoalcohols and aldehydes.

In an embodiment, the vector is a lentiviral vector.

### Drug, Preparation and Composition Thereof

The disclosure provides a preparation comprising the CAR-T cells of the disclosure and a pharmaceutically acceptable carrier, diluent or excipient. In an embodiment, the preparation is a liquid preparation. In an embodiment, the preparation is an injection. The concentration of CAR-T cells in the preparation is in a range of 1×10³-1×10⁸ cells/ml, or in a range of 1×10⁴-1×10⁷ cells/ml.

In an embodiment, the formulation may include a buffer such as neutral buffered saline, sulfate buffered saline; carbohydrates such as glucose, mannose, sucrose or dextran, mannitol; proteins; polypeptides or amino acids such as glycine; antioxidants; chelating agents such as EDTA or glutathione; adjuvants (e.g., aluminum hydroxide); and preservatives. The preparations of the disclosure can be formulated for intravenous administration.

In certain embodiments, the pharmaceutical composition of the disclosure comprises: the modified immune cell or immune cell composition as disclosed herein.

The isolated nucleic acid molecules, vectors, host cells, modified immune cells or immune cell compositions of the disclosure can be prepared into any dosage forms known in the medical field, such as tablets, pills, suspensions, emulsions, solutions, gels, capsules, powders, granules, pulvises, lozenges, suppositories, injections (including injection, sterile powder for injection and concentrated solution for injection), inhalants and sprays. The dosage form depends on the intended mode of administration and therapeutic use. The pharmaceutical composition of the disclosure should be sterile and stable under production and storage conditions. An example dosage form is injection. Such injections can be sterile injection solutions. In addition, the sterile injection solution can be prepared into sterile freeze-dried powder (for example, by vacuum drying or freeze drying) for convenient storage and use. Such sterile freeze-dried powder can be dispersed in a suitable carrier before use, such as water for injection (WFI), bacteriostatic water for injection (BWFI), sodium chloride solution (e.g. 0.9% (w/v) NaCl), glucose solution (e.g. 5% glucose), solution comprising surfactant (e.g. 0.01% polysorbate 20), pH buffer solution (e.g. phosphate buffer solution), Ringer's solution and any combination thereof.

The isolated nucleic acid molecules, vectors, host cells, modified immune cells or immune cell compositions of the disclosure can be administered by any suitable method known in the art, including but not limited to oral, buccal, sublingual, eyeball, local, parenteral, rectal, intrathecal, cisternal, inguinal, intravesical, topical (such as powder, ointment or drops) or nasal route. However, for many therapeutic purposes, a route/mode of administration can be parenteral administration (for example, intravenous injection or infusion, subcutaneous injection, intraperitoneal injection, intramuscular injection). Those skilled in the art will understand that the route and/or mode of administration will vary according to the intended purpose. In some embodiments, the isolated nucleic acid molecule, nucleic acid construct, vector, host cell, modified immune cell or immune cell composition of the disclosure is administered by intravenous injection or infusion.

The pharmaceutical composition of the disclosure may include a therapeutically effective amount or a prophylactically effective amount of the isolated nucleic acid molecule, nucleic acid construct, vector, host cell, modified immune cell or immune cell composition of the disclosure. “Prophylactically effective amount” refers to an amount sufficient to prevent, stop or delay the occurrence of diseases. “Therapeutically effective amount” refers to an amount sufficient to cure or at least partially prevent the disease and its complications of patients who already have the disease. The therapeutically effective amount of the isolated nucleic acid molecule, nucleic acid construct, vector, host cell, modified immune cell or immune cell composition of the disclosure may vary according to the following factors: the severity of the disease to be treated, the overall state of the patient's own immune system, the general situation of the patient such as age, weight and sex, the mode of drug administration, other treatments administered at the same time, and the like.

In the disclosure, the administration regimen can be adjusted to obtain the best desired response (such as therapeutic or prophylactical response). For example, the drug can be administered once, can be administered multiple times over a period of time, or the dose can be reduced or increased in proportion to the urgency of the treatment situation.

### Therapeutic Application

The therapeutic application of the disclosure includes collecting T cells of patients, and transducing CAR gene with lentivirus (four-plasmid system package) to obtain BCMA-CD19 CAR-T cells, so that the BCMA-CD19 CAR-T cells can specifically recognize the target cells with BCMA and CD19 antigens expressed on the cell surface, thus carrying out therapeutic application.

Therefore, the disclosure further provides a method for stimulating a T cell-mediated immune response to a target cell population or tissue of a mammal, which comprises the following steps: administering the CAR-T cells of the disclosure to the subject (e.g., a mammal, a human) in need of treatment. The subject may have or be suspected of having an autoimmune disease and/or a caner. One suspected of having an autoimmune disease may exhibit symptoms commonly associated with the disease. A suspicion of disease in an individual may also be determined based upon physical examination of the subject by a qualified professional, results of one or more genetic and/or protein assays that could indicate a disease condition, and/or one or more risk factors of the subject. In some instances, the autoimmune disease is a B-cell mediated autoimmune disease.

In an embodiment, the disclosure includes a kind of cell therapy, in which autologous T cells (or heterologous donors) of patients are isolated, activated and genetically modified to produce CAR-T cells, and then the cells are injected into the same patient. In this way, the probability of graft-versus-host disease is extremely low, and the antigen is recognized by T cells without MHC limitation.

In an embodiment, the CAR-T cells of the disclosure can undergo stable T cell expansion in vivo and can last for an extended amount of time. In addition, the CAR-T mediated immune response can be part of the adoptive immunotherapy step, in which CAR-T modified T cells induce an immune response specific to the antigen-binding domain in CAR. For example, CAR-T cells that are resistant to BCMA and/or CD19 cause specific immune responses against cells that express BCMA and/or CD19.

Although the data disclosed herein specifically disclose lentiviral vectors including anti-BCMA and/or CD19scFv, hinge and transmembrane regions, and 4-1BB/CD28 and CD3ζ signal transduction domains, the disclosure should be interpreted as including any number of changes to each of the components of the construct.

The drug can kill or inhibit CD19-positive B cells, plasmablasts, and BCMA-positive plasma cells to treat autoimmune diseases. B cells are associated with many autoimmune diseases. Autoreactive B cells, and hyperactive or over stimulated B cells play a role in autoimmune diseases. In some instances the autoimmune diseases may have aberrant B cell maturation and/or autoantibody production. B cell mediated autoimmune diseases are marked by the loss of self-tolerance during the development of B cells. Thus, B cells are important to autoimmune disease pathology. Examples of B cell mediated autoimmune diseases are discussed below. Each of these diseases may be treated by administration of a composition, formulation, or pharmaceutical composition disclosed herein to a subject in need of treatment. The subject may have or be suspected of having an autoimmune disease.

The autoimmune disease can be an autoimmune disease mediated by B cells. The autoimmune diseases mediated by B cells include systemic lupus erythematosus (SLE), glomerulonephritis includes autoimmune chronic kidney disease, lupus nephritis, acute glomerulonephritis, immune nephritis, membranous nephropathy, IgA nephropathy, Anti-Neutrophil Cytoplasmic Antibody (ANCA) Associated Vasculitis (AAV); scleroderma or systemic sclerosis (SSc); myositis or idiopathic inflammatory myositis including dermatomyositis, polymyositis, immune-mediated necrotizing myopathy (IMNM), antisynthetase syndrome, inclusion body myositis, and overlap myositis; multiple sclerosis (MS); inflammatory bowel disease (IBD); rheumatoid arthritis (PA); Sjogren's syndrome (SS); autoimmune hemolytic anemia; neuromyelitis optica (NMO); neuromyelitis optica spectrum disease (NMOSD); idiopathic thrombocytopenic purpura (ITP); systemic autoimmune small vessel vasculitis syndrome or polyangiitis related to antineutrophil cytoplasmic antibody; Wegener's granulomatosis (GPA), eosinophilic granulomatosis with polyangiitis (EGPA, Churg-Strauss syndrome); pemphigus vulgaris; autoimmune encephalitis; pemphigus vulgaris; myasthenia gravis; antiphospholipid syndrome; Chagas' disease; Graves' disease; polyarteritis nodosa; pulmonary hemorrhage-nephritis syndrome; Kawasaki disease, amyloidosis; monoclonal immunoglobulin of undetermined significance, POEMS syndrome; Crohn's disease; ulcerative colitis; adult onset Still's disease; and chronic progressive cortical demyelinating encephalopathy (CIDP).

As mentioned above, these diseases are B cell autoimmune diseases. Many of these diseases are due to the deposition of immune complexes in afflicted tissue, inflammation, or other attack by autoantibodies that damages cells/tissues. The pathology of some of the above-mentioned B cell mediated autoimmune diseases is further discussed below.

SLE, as mentioned earlier, is an autoimmune disease that can affect multiple organs. SLE is the most common type of lupus. The symptoms of SLE are heterogeneous, affecting multiple different organs and having varying severity between patients. SLE includes moderate to severe refractory SLE, lupus nephritis, active lupus nephritis and active systemic lupus erythematosus without renal involvement. Refractory (or severe) SLE may include patients who have received one or more previous drug treatments for an autoimmune disease but who show no or poor response despite the drug treatment.

SLE is characterized by an immune response against endogenous nuclear (e.g., nucleic acids and nuclear proteins) and cytoplasmic material. The production of autoantibodies (e.g., ANA) is a hallmark of SLE and it is driven by autoreactive B cells. The immune response activated by the autoantibodies may result in inflammation. Thus, B cells play a critical role in the pathogenesis of SLE and SLE may be treated and/or prevented with a composition, formulation, or pharmaceutical compositions disclosed herein.

Glomerulonephritis (GN) refers to the pathogenesis involved in autoimmune chronic kidney diseases (CKD). Autoimmune CKD are associated with autoantibody production by B-cells and can include membranous nephropathy, IgA nephropathy and Anti-Neutrophil Cytoplasmic Antibody (ANCA) Associated Vasculitis (AAV). GN is an inflammation of the glomeruli of the kidney. CKD refers to a reduced ability or inability for the kidneys to filter blood and can be due to antibody-related injury to the glomerulus. Lupus nephritis (discussed below) is a type of GN caused by SLE. That is, the LN is a secondary effect due to SLE. Membranous nephropathy (discussed below) is another type of GN that may occur as a secondary effect due to SLE or as a primary effect (e.g., without a related disease such as SLE). GN involves a loss of tolerance and adaptive immune response to self-antigens. In particular, immune complexes directed towards IgA and/or IgG circulating in the blood. IgA nephropathy, for example, is characterized by IgA autoantibodies being deposited in the glomeruli. Autoantibodies are a hallmark of the disease pathology for GN. Thus, depleting B cells with a composition, formulation, or pharmaceutical composition disclosed herein can prevent and/or treat GN.

Lupus nephritis (LN) is common consequence of SLE. ANAs can form immune complexes that may be deposited in glomeruli or can react with the glomerular basement membrane of the kidney. Lupus nephritis is associated with a higher morbidity and may progress to end stage renal disease. Consequently, preventing or reducing progression of LN is vital. As LN itself is a progression of SLE, it too is a B-cell mediated autoimmune disease.

Membranous nephropathy (MN) is an autoimmune disease characterized by a thickening of the glomerular capillary walls due to immune complex deposition. MN occurs in all regions and all ethnicities, with increased prevalence among east Asian populations, with approximately 25% of all primary GNs in China are MN. The annual incidence rates of MN are estimated at 10-12 per million in North America and 2-17 per million in Europe. The disease affects individuals of all ages with a mean age of diagnosis at 50-60 years and a 2:1 male predominance for unknown reasons. MN is the most common cause of idiopathic nephrotic syndrome in non-diabetic adults worldwide, accounting for 20-37% in most kidney biopsy series and increasing to as high as 58% in adults >65 years of age. MN is uncommon in children. The natural history of untreated MN has been reported with spontaneous complete remission rates of 20-30% and 10-year renal survival rates of 60-80%. In patients who continue to have nephrotic syndrome, kidney failure develops in 40-50% over a period of 10 years. These patients are also at an increased risk of life-threatening thromboembolic and cardiovascular events. Antibodies to PLA2R are specific for MN and found in ˜70% of adult patients with the disease. The podocyte is both the target of circulating auto-antibodies and probably the main source of the auto-antigen. Several further antigens have been identified, including thrombospondin type 1 domain-containing 7A (THSD7A) which accounts for <5% of MN. The current standard of care treatment in MN involves treatment with one or more of prednisolone, cyclophosphamide, calcineurin inhibitor, and/or rituximab. However, none of these treatments deplete the antibody producing B cells.

Multiple sclerosis (MS) is a chronic disease that may be caused by autoantibodies attacking the central nervous system, in particular the myelin of nerve fibers. There are four main types of MS-relapsing-remitting MS, secondary-progressive MS, primary-progressive MS, and progressive-relapsing MS. Each form is typically defined by frequency and severity of the symptoms associated with an attack. MS can affect vision, cognition, mood, muscle movements, and bladder control. B cells can be involved in the pathogenesis of MS by antibody secretion by plasmablasts and plasma cells as well as (i) antigen presentation to T cells and driving auto proliferation of brain-homing T cells (presumably by memory B cells), (ii) production of pro-inflammatory cytokines and chemokines that propagate inflammation, (iii) production of soluble toxic factors contributing to oligodendrocyte and neuronal injury, (iv) contribution to the formation of ectopic lymphoid aggregates in the meninges, and (v) providing a reservoir for Epstein-Barr (EBV) virus infection (see Comi et al., Ann. Neurol., Vol. 89(1), pp. 13-23, January 2021). Depletion of B cells has been successful in the treatment of relapsing MS and primary progressive MS patients. Id. Thus, the CAR compositions, formulations, or pharmaceutical compositions disclosed herein may be used to treat or prevent MS in a patient having or suspected of having MS.

Myositis (idiopathic inflammatory myopathy) refers to a group of autoimmune diseases characterized by weakening, swelling, pain, and/or inflammation of the skeletal muscles. The most common forms of myositis are polymyositis and dermatomyositis. Other forms include immune-mediated necrotizing myopathy (IMNM), antisynthetase syndrome, inclusion body myositis, and overlap myositis. Dermatomyositis primarily affects skin, muscles, joints, and lungs. Skin lesions are commonly associated with the disease and may precede muscle symptoms. Polymyositis is associated with muscle weakness and is primarily diagnosed by exclusion of other maladies. The involvement of B cells in myositis is based on the presence of autoantibodies, which B cells produce. Myositis is regularly associated with myositis-specific autoantibodies (MSA) or myositis associated autoantibodies (MAA). BAFF has been implicated in the production of these autoantibodies in myositis patients. Consequently, depletion of B cells with one of the compositions, formulations, or pharmaceutical compositions disclosed herein in patients having or suspected of having myositis is desirable to treat and/or prevent the disease.

Myasthenia gravis (MG) affects the neuromuscular junction of skeletal muscles. It typically involves the muscles of the eyes, throat and extremities and manifests as muscle weakness. MG is an autoimmune disease in which autoantibodies to one or more of nicotinic acetylcholine receptors, muscle-specific kinase, and lipoprotein-related protein 4 are found in the majority of patients. The production of autoantibodies indicates a role for B cells in the pathology of MG (see Yi et al., Muscle Nerve, vol. 57(2), pp. 172-184, February 2018).

Scleroderma or Systemic sclerosis (SSc) is a connective tissue disorder that can be local or diffuse (e.g., systemic). It typically manifests by a thickening of the skin. ANAs are present in approximately 90% of cases. Autoantibodies to the centromere, SCL70, and RNA polymerase III are also prevalent in a majority of cases. Systemic sclerosis can affect multiple organs including skin, gastrointestinal tract, lungs, kidneys, skeletal muscle, and pericardium. Activated B cells produce autoantibodies that directly contribute to the pathology. For in vitro immunization, at least one of the following occurs in vitro before the cells are administered into mammals: i) expanding the cells, ii) introducing the nucleic acid encoding CAR into the cells, and/or iii) cryopreserving the cells.

Amyloidosis is a disease in which amyloid deposits build up in a person's organ(s) (e.g., heart, brain, kidney, stomach, intestine, and spleen). There are three types of amyloidosis. Light chain (AL) amyloidosis occurs when plasma cells produce an excess of abnormal light chain proteins that accumulate in tissues and organs. The plasma cells may be targeted by the bispecific CAR disclosed herein. Other forms of amyloidosis include serum amyloid A protein (AA) amyloidosis and transthyretin (ATTR) amyloidosis.

Ex vivo procedures are well known in the art and are discussed more fully below. Briefly, cells are isolated from mammals (e.g., humans) and genetically modified (i.e., transduced or transfected in vitro) with a vector expressing CAR disclosed herein. CAR-T modified cells can be administered to mammalian recipients to provide therapeutic benefits. The mammalian recipient can be human, and the CAR-T modified cells can be autologous relative to the recipient. Optionally, the cells may be allogeneic, syngeneic or heterogeneous with respect to the recipient.

The disclosure provides a method for treating tumors, which comprises administering a therapeutically effective amount of CAR-T modified T cells of the disclosure to a subject in need thereof.

A formulation, composition, or pharmaceutical composition containing a CAR as disclosed herein may be administered at a dosage of 0.5±20%×10\hat{ }5 cells/kg, 1±20%×10\hat{ }5 cells/kg, 2±20%×10\hat{ }5 cells/kg, or 3±20%×10\hat{ }5 cells/kg. A single arm clinical trial for SLE has been initiated. See NCT05858684 for the detailed enrollment criteria. In an embodiment, the dosage is 0.5×10\hat{ }5 cells/kg, 1×10\hat{ }5 cells/kg, 2×10\hat{ }5 cells/kg, or 3×10\hat{ }5 cells/kg. The above-mentioned dosages may be administered to a patient having or suspected of having an autoimmune disease. The autoimmune disease can be a B cell mediated autoimmune disease.

The CAR-T modified T cells of the disclosure can be administered alone or as a pharmaceutical composition in combination with diluents and/or other components such as IL-2, IL-17 or other cytokines or cell populations. Briefly, the pharmaceutical composition of the disclosure may include the target cell population as described herein in combination with one or more pharmaceutically or physiologically acceptable carriers, diluents or excipients. Such compositions may include buffers such as neutral buffered saline, sulfate buffered saline; carbohydrates such as glucose, mannose, sucrose or dextran, mannitol; proteins; polypeptides or amino acids such as glycine; antioxidants; chelating agents such as EDTA or glutathione; adjuvants (e.g., aluminum hydroxide); and preservatives. The compositions of the disclosure are formulated for intravenous administration in an embodiment.

The pharmaceutical composition of the disclosure can be administered in a manner suitable for the disease to be treated (or prevented). The number and frequency of administration will be determined by such factors as the patient's condition and the type and severity of the patient's disease, although the appropriate dose can be determined by clinical trials.

### EXAMPLES

### Example 1: Differentiation of Antibody-Secreting Cells in Vitro

Day0-Day3: After B cells were isolated, an appropriate amount of the cells was counted and detected for cell phenotype by flow cytometry. The remaining cells were re-suspended to a density of 0.5M/mL with freshly prepared B cell basal medium (IMDM+Glutamax+10% FBS), and then transferred to a 24-well plate covered with feeder cell layer (irradiated L cells expressing human CD40L, 0.125M/well), and cytokines IL-2 (20 U/mL) and IL-21 (50 ng/mL) and antibody F(ab′)2 goat anti-human IgM and IgG (10 μg/mL) were added according to their respective concentrations.

Day3-Day6: B cells cultured to Day3 were transferred out of the plate, and an appropriate amount of the cells was counted and detected for cell phenotype by flow cytometry. After counting, the cells were centrifuged at 400 g for 5 min, the supernatant was discarded and then the cells were re-suspended in B cell basal medium (IMDM+Glutamax+10% FBS) to a density of 0.2M/mL. At the same time, cytokines IL-2 (20 U/mL) and IL-21 (50 ng/mL), as well as hybridoma growth additive HyMAX™ Hybridoma Fusion & Cloning Supplement with a final concentration of 1×, Lipid Mixture 1 and MEM amino acids solution were added for continuous culture.

Day6-Day13: When cultured to Day6, an appropriate amount of cells was counted and detected for phenotype by flow cytometry. After counting, the cells were centrifuged at 400 g for 5 min, the supernatant was discarded and then the cells were re-suspended in B cell basal medium (IMDM+Glutamax+10% FBS) to a density of 0.5M/mL, and then transferred to a 6-well plate covered with bone marrow stromal cells (irradiated M2-10B4 cells, 0.2M/well), 4 mL per well. At the same time, cytokines IL-6 (10 ng/mL), IL-21 (50 ng/mL) and IFN-α (100 U/mL) according to their respective concentrations, as well as hybridoma growth additive HyMAX™ Hybridoma Fusion & Cloning Supplement, Lipid Mixture 1 and MEM amino acids solution were added for further culture. Half of the medium was exchanged on Day10, and culture was continued until Day12 to Day13 for use in in vitro killing assay of target cells.

### Example 2: Flow Cytometry Staining for Car Expression

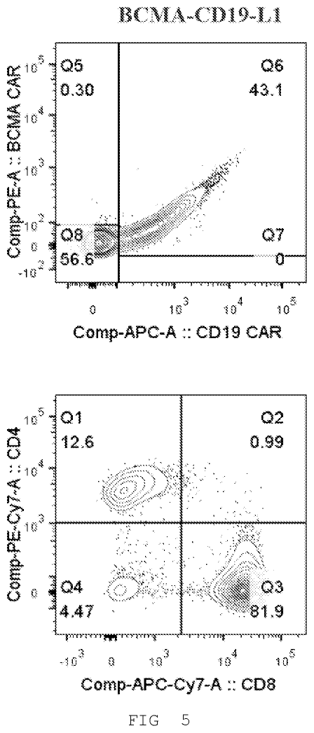

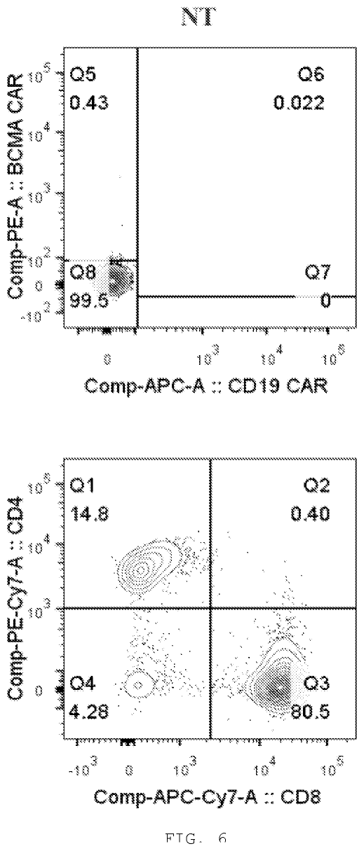

Some cells in culture were taken and well mixed, 1×10⁵-5×10⁵ cells were taken, centrifuged at 300×g for 5 min, and the supernatant was discarded;
The concentration of BCMA-his antigen was adjusted to 10 μg/mL (1:10 dilution) with FACS buffer, 100 μL of the prepared primary antibody was taken to re-suspend the cell sample, and the system was allowed to stand at 4° C. for staining for 50 min;
The cells were washed with FACS buffer once after staining was completed;
Anti-His Tag-PE, Rabbit Anti-Mouse FMC63 scFv Monoclonal Antibody Alexa Fluor 647 (Clone: R19M), BV421-anti-CD3, PE-Cy7 anti-CD4, APC-H7 anti-CD8 were diluted at 1:100 to prepare a mixed solution of secondary antibodies, and then 100 μL was taken to re-suspend the cell sample, the system was well mixed and then allowed to stand at 4° C. in the dark for staining for 25 min;
After staining was completed, the cells were washed twice with FACS buffer, re-suspended with 7-AAD buffer diluted at 1:300, and detected on the machine. The results are as shown in FIGS. 3-6 (in which CD19 scFv and BCMA scFv in CD19 CAR-T and BCMA CAR-T are the same as the corresponding protein sequences in BCMA-CD19-L1 dual-target CAR-T). It can be seen that high-level expression of both anti-CD19 and anti-BCMA antibodies can be achieved on the surface of BCMA-CD19 CAR-T cells according to the present application.

### Example 3: Flow Cytometry Staining of Antibody-Secreting Cells

Some cells in culture were taken and well mixed, 1×10⁵-5×10⁵ cells were taken, centrifuged at 300×g for 5 min, and the supernatant was discarded;
A clean 1.5 ml EP tube was taken, a proper amount of FACS buffer was added according to the sample size, and then the following antibodies were added at 1:100: APC-anti-CD19, PE-anti-BCMA, BUV395-anti-CD27, FITC-anti-CD38, BV421-anti-CD138 and BV605-anti-CD20, which were well mixed to obtain the antibody staining fluid.

The antibody-secreting cells in culture were taken out on Day0, Day3, Day6, Day10, Day13 and Day14, respectively, well mixed, an appropriate amount of cells was taken, centrifuged at 400 g for 5 min, and the supernatant was discarded.

The cells were re-suspended with the prepared staining fluid and stained at 4° C. in the dark for 25 min, washed with FACS buffer once, centrifuged at 400 g for 5 min, and the supernatant was discarded.

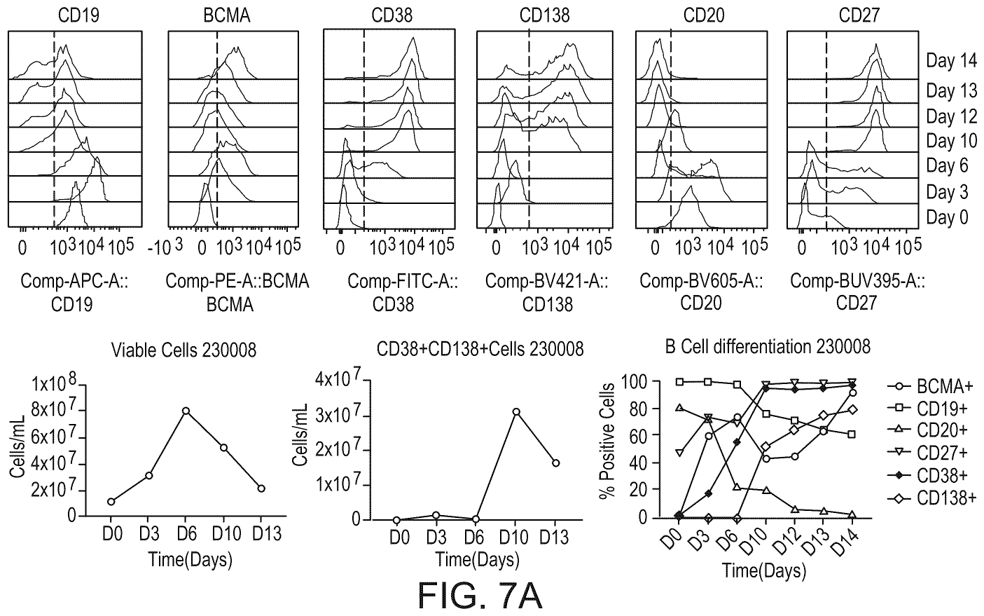

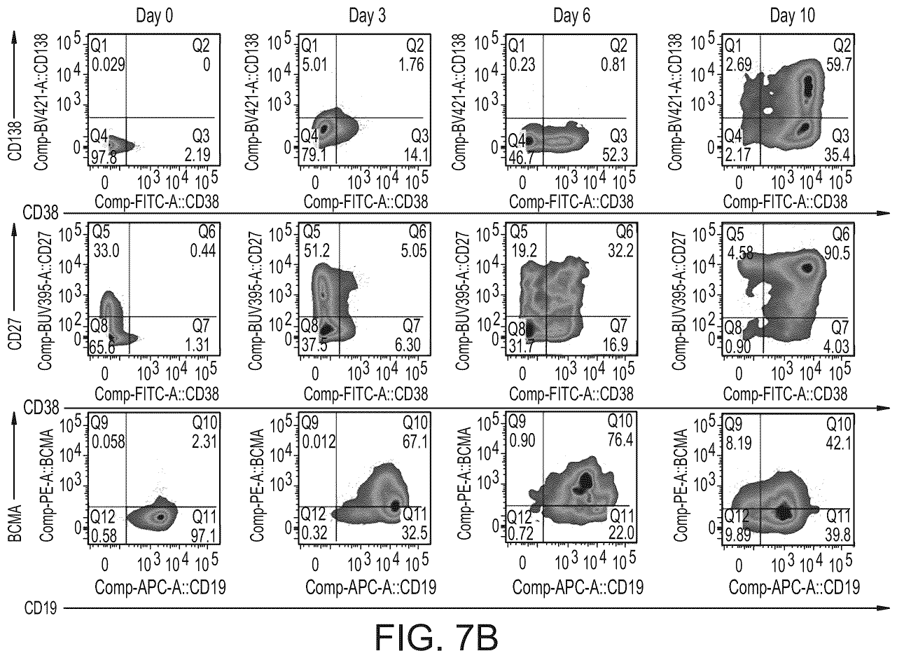

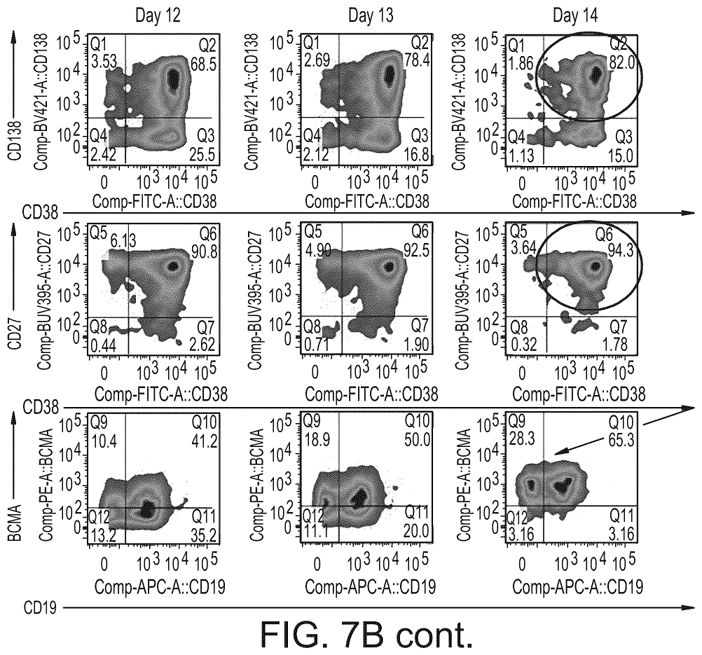

The cells were washed with FACS buffer once again, centrifuged at 400 g for 5 min, and the supernatant was discarded;
The cells were re-suspended with FACS buffer, and immediately tested on the machine. The change of absolute number of different cell subsets on different days of differentiation, and the change of expression amount and percentage of CD19, BCMA, CD20, CD27, CD38 and CD138 on the cell surface are shown in FIG. 7A. FIG. 7B shows the expression level of different antigens on the cell surface and the protein expression on the cell surface during the 14-day differentiation cycle. At the end of differentiation, the proportion of plasma cells that were CD38 and CD138 positive was 82%, and the proportion of antibody-secreting cells that were CD38 and CD27 positive was 94.3%. At the end of differentiation, 31.5% of the cells were CD19 negative. It can be seen that after the end of differentiation, antibody-secreting cells with high purity were obtained, and a certain proportion of these cells were CD19 negative.

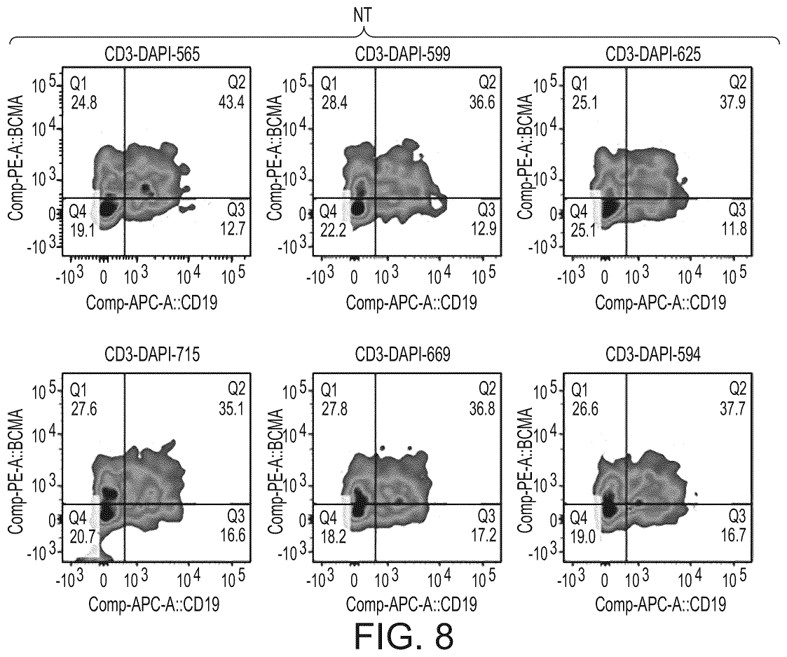

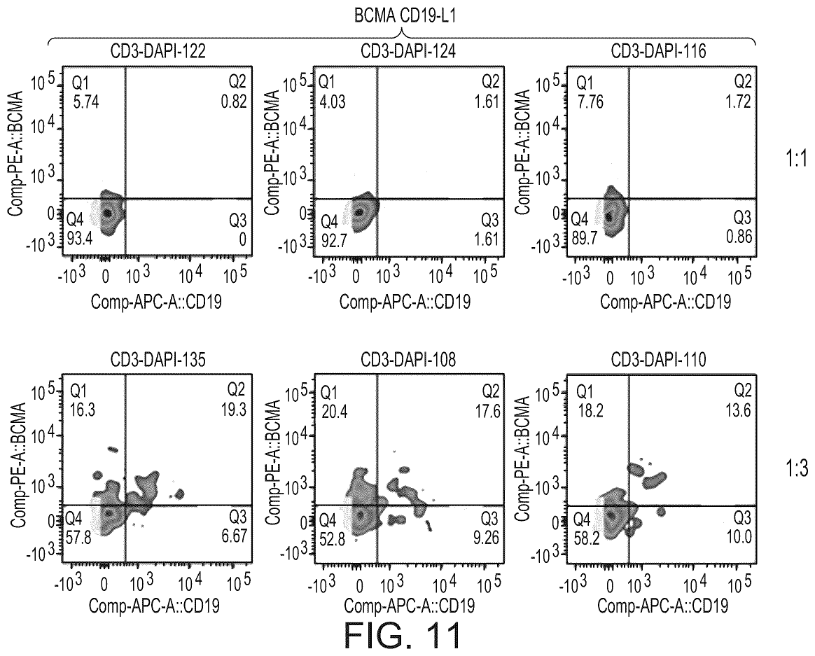

After killing antibody-secreting cells that have been differentiated in vitro with NT, CD19 CAR-T, BCMA CAR-T and BCMA-CD19 dual-target CAR-T for 5 h, the flow cytometry experiment was carried out to detect the absolute number of residual live antibody-secreting cells after killing, so as to detect the killing ability of three kinds of CAR-T to total antibody-secreting cells. The results are shown in FIGS. 8-12, from which it can be seen that BCMA-CD19 dual-target CAR-T can clear CD19+ and CD19-antibody-secreting cells simultaneously.

### Example 4: ELISPOT of Antibody-Secreting Cells

### Day 0: (Aseptic Operation is Required)

Millipore 96-well PVDF ELISPOT plate was taken out, and 25 μL of 70% ethanol was added to each well for treatment at room temperature for 1 min;
The plate was washed with PBS twice, 200 μL per well each time, the wash buffer was discarded after the final wash, and the plate was gently knocked dry on sterile paper;
50 μL of the diluted coated antibody or specific antigen was added to each well, and the system was incubated overnight at 4° C.;

### Day 1: (Aseptic Operation is Required)

The coated antibody or antigen was removed from the plate, and the plate was washed with sterile PBS for 3 times, with 200 μL per well each time; the wash buffer was discarded after the final wash, and the plate was gently knocked dry on sterile paper;
200 μL of blocking buffer (1×) was added to each well to block at room temperature for at least 1 h;
The blocking buffer was removed (without washing the plate), the prepared cells in each group was added, 100 μL of the cell suspension for each well;
After adding the cells, the edge of the plate was gently patted to spread the cells evenly, the plate was put into an incubator, and cultured at 37° C. and 5% CO₂ for 16-20 h (be careful not to move the plate during the culture);

### Day 2: (Aseptic Operation is not Required)

The cells in the plate were removed, and the plate was washed with PBS twice to completely remove the cells, 200 μL per well each time;
The plated was washed with wash buffer for 5 times, 250 μL per well each time;
100 μL of diluted detection antibody was added to each well, and the system was incubated at room temperature for 2 h or at 4° C. overnight;
The detection antibody was removed, and the plate was washed with wash buffer for 5 times, 250 μL per well each time;
100 μL of diluted conjugate was added to each well, and the system was incubated at room temperature in the dark for 1 h.

The conjugate was removed, and the plate was washed with wash buffer for 5 times, 250 μL per well each time;
100 μL of freshly prepared AEC chromogenic solution was added to each well and the system was incubated for 30 min at room temperature in the dark until clear spots appeared;
The chromogenic solution was removed, and color development was stopped with deionized water, 50 μL per well each time;
Excess liquid was removed from the plate, and the plated was placed in the dark at room temperature to dry thoroughly;
Spots were counted using ELISPOT reader. Antibody-secreting cells differentiated in vitro were killed by NT, CD19 CAR-T, BCMA CAR-T and BCMA-CD19 dual-target CAR-T for 5 h in vitro, and then the ELISPOT experiment was carried out to detect the absolute number of cells that can secrete IgG in the remaining cells, so as to detect the killing ability of the three CAR-T to the total antibody-secreting cells. At the end of the experiment, the experiment was photographed and analyzed by CTL enzyme-linked fluorescence reader. The photos showing the absolute number of spots are shown in the figure, and the statistical analysis of absolute number of spots is as follows. The results showed that the BCMA-CD19 dual-target CAR-T group had the least residual total antibody secreting cells. Results as shown in FIG. 8. As can be seen from the results of FIG. 8, BCMA-CD19 CAR-T cells can clear more antibody-secreting cells than CD19-CAR-T and BCMA-CAR-T cells, and have stronger killing ability.

### Example 5: Killing Assay for B Cells and Antibody-Secreting Cells In Vitro

The concentrations of primary B cells isolated from PBMC and antibody-secreting cells differentiated in vitro were 4×10⁵ cells/mL and 5×10⁵ cells/mL, respectively.

Effector cells were prepared, the concentration of CAR positive cells was adjusted to 12×10⁵ cells/mL and 5×10⁵ cells/mL, and the CAR positive cells were diluted in turn according to the planned effector to target (E/T) ratio;
A clean 96-well plate was taken and the experimental groups were marked. Each group was provided with three parallel wells, and 100 μL of the target cell suspension and 100 μL of the effector cell suspensions were added into the corresponding marked wells successively, with a total volume of 200 μL;
The effector cells and target cells were co-incubated in a 37° C. incubator for 5 h;
After incubation for 5 h, the cells were centrifuged at 300 g for 5 min, and 50 μL×2 samples were taken from each well for CBA detection;
Then, 1 μL of FITC-anti-CD3, 1 μL of APC-anti-CD19 and 1 μL of PE-anti-BCMA antibodies were added to each original cell well, and the system was mixed evenly and stained at 4° C. for 25 min; after staining, the cells were washed with FACS buffer once, then re-suspended with 120 μL of DAPI diluent (1:4800 V/V), stained for 5 min at room temperature in the dark, and immediately detected on the machine.

Samples with equal absolute volume were collected from each well to analyze the absolute number of living B cells and antibody-secreting cells in the residual samples.

### Data Processing:

The percentage of specific killing of ASC was calculated according to the following formula: killing percentage (%) of CAR-T cells=(absolute number of CD3⁻DAPI⁻ cells in the target cell group-absolute number of CD3⁻DAPI⁻ cells in the CAR-T and target cell co-incubation group)/absolute cell number of CD3-DAPI− in the target cell group.

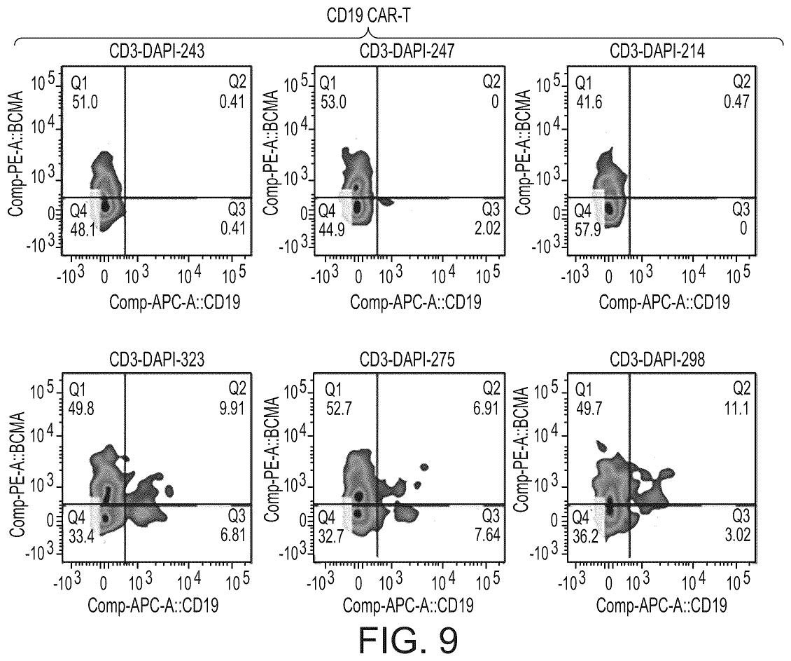

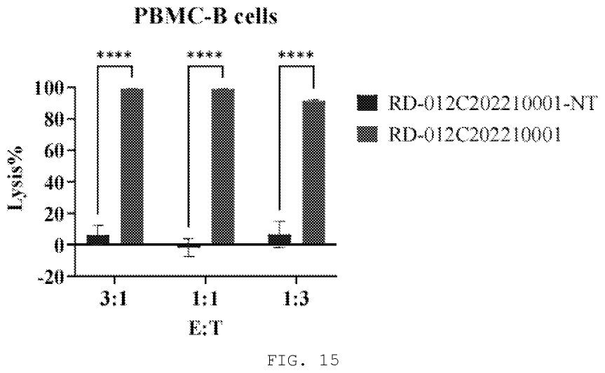

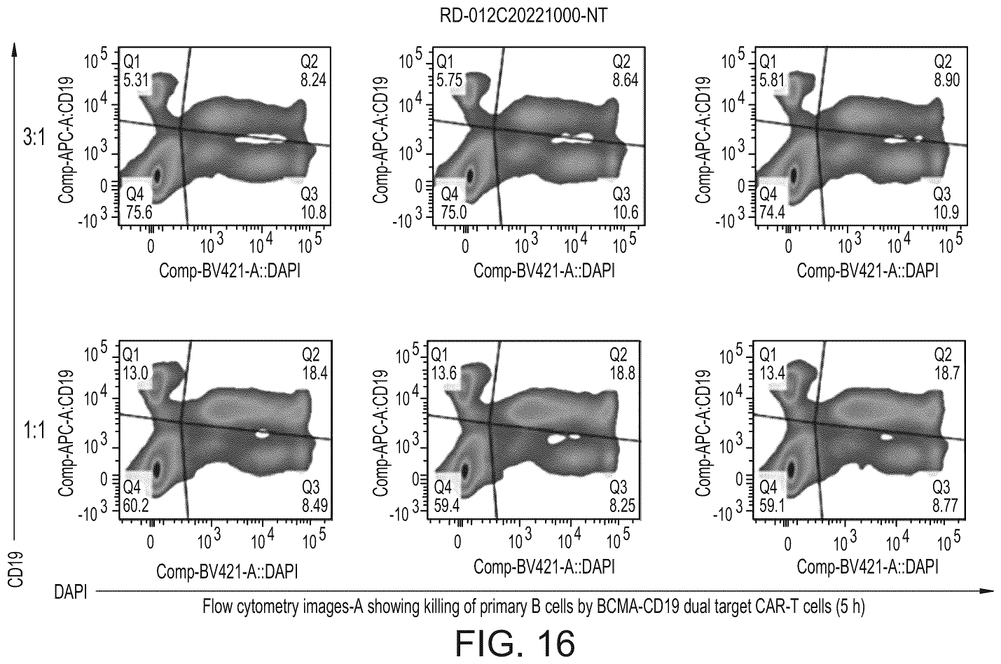

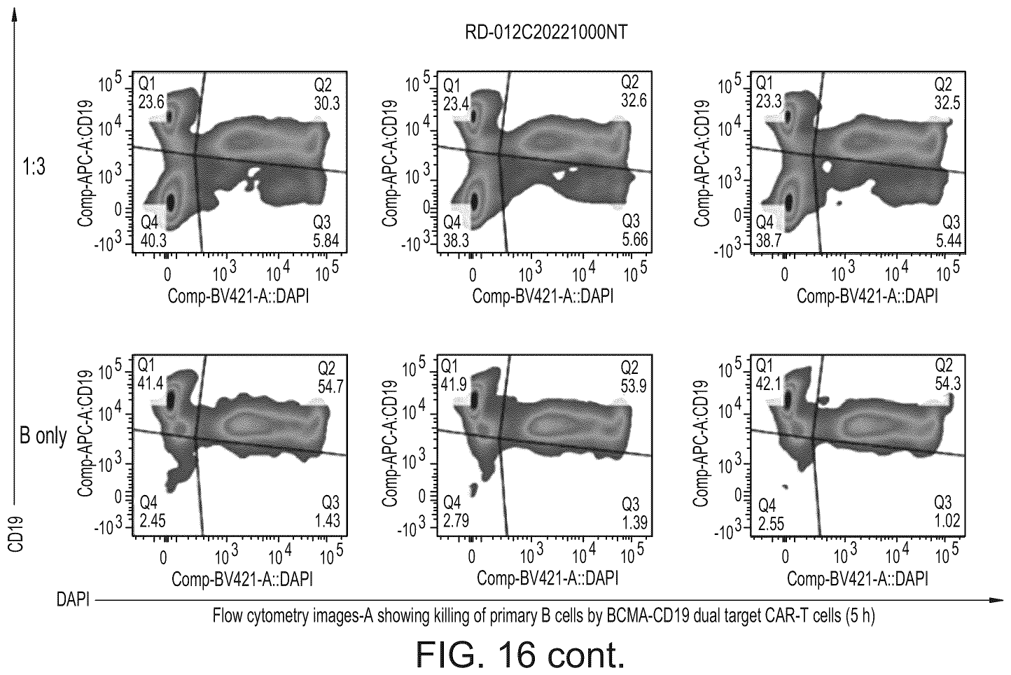

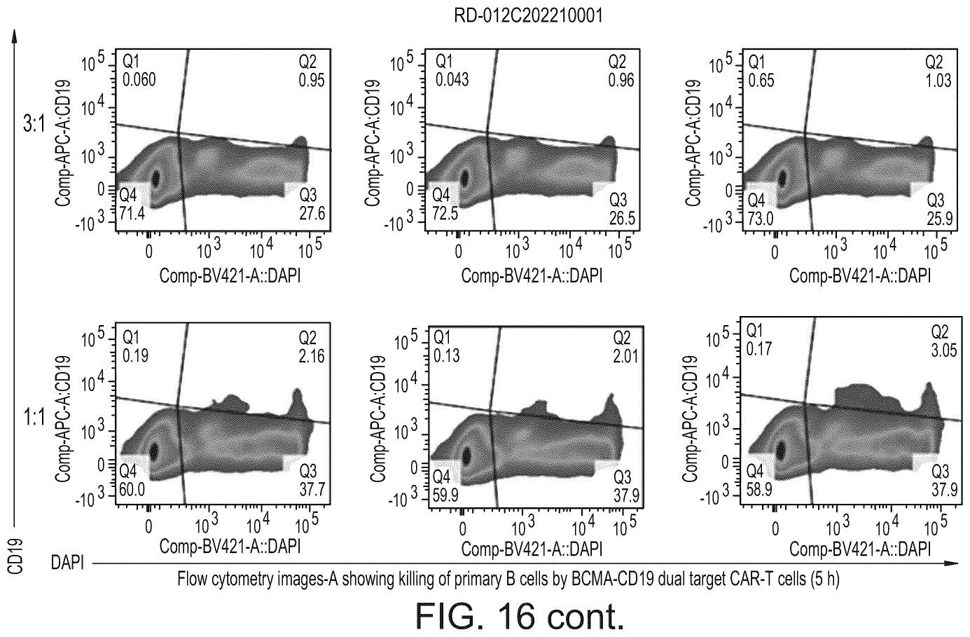

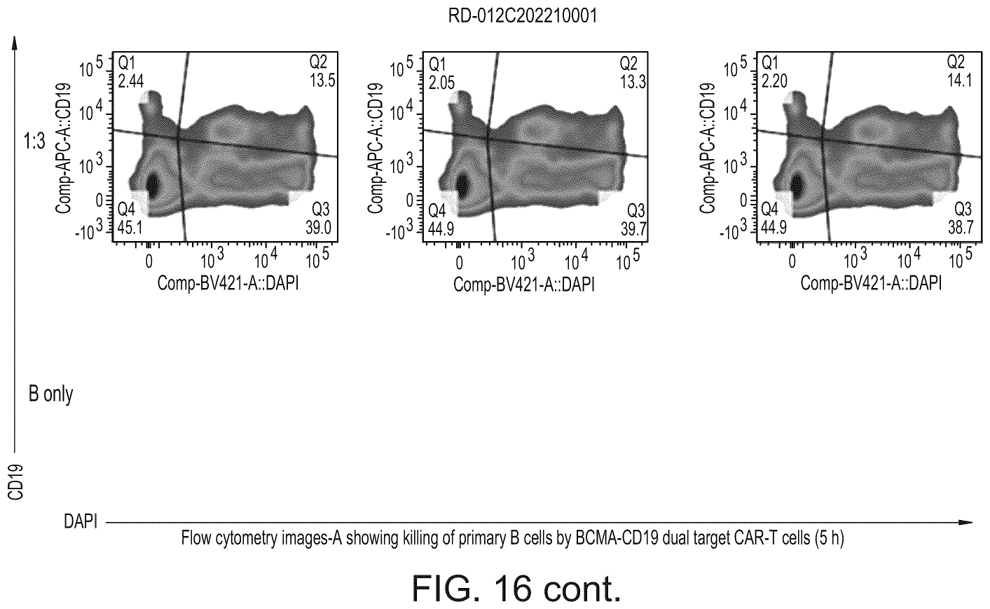

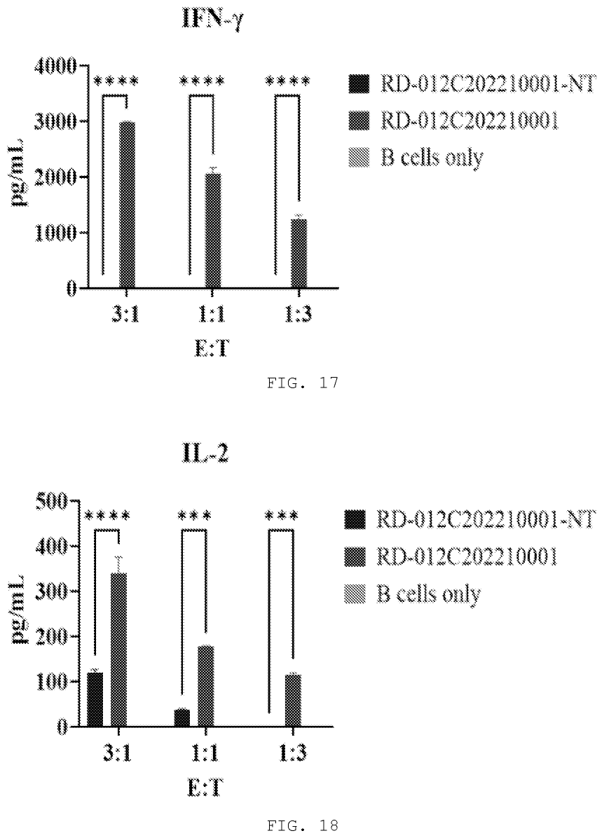

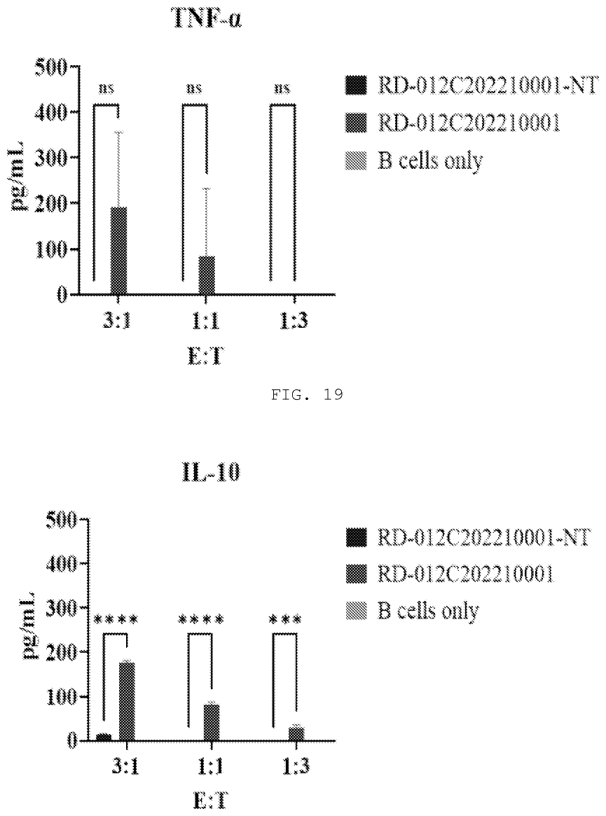

The percentage of specific killing of B cells was calculated according to the following formula: killing percentage (%) of CAR-T cells=(absolute number of CD19⁺DAPI⁻ cells in the target cell group-absolute number of CD19⁺DAPI⁻ cells in the CAR-T and target cell co-incubation group)/absolute number of CD19⁺DAPI⁻ cells in the target cell group. The results are shown in Tables 3-5 and FIGS. 9-20. From the results in Tables 3-5 and FIGS. 9-20, BCMA-CD19 CAR-T cells have stronger killing ability on primary B cells and antibody-secreting cells; the secretion level of cytokines in primary B cells and antibody-secreting cells after killing is significantly lower than that of CD19-CAR-T and BCMA-CAR-T.

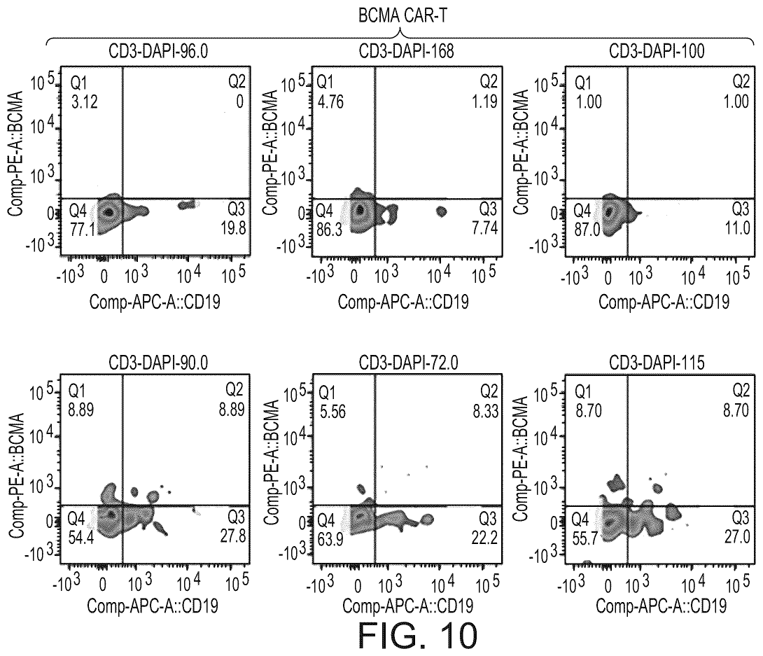

The experiment shown in FIG. 10 is as follows: B cells were isolated and purified from PBMC, co-incubated with BCMA-CD19 dual-target CAR-T cells for 5 h in vitro, and then the absolute number of remaining living B cells was detected, and the ability of BCMA-CD19 dual-target CAR-T to kill B cells in vitro was statistically analyzed. In the figures, RD-012C20221001 (BCMA-CD19-L1) is BCMA-CD19 dual-target CAR-T cells, and RD-012C20221001-NT is the T cell control from the same donor source. See FIG. 10 for the specific killing values of each group. The results show that BCMA-CD19 dual-target CAR-T can effectively clear primary B cells in vitro.

| | Group | | | | | |
|---|---|---|---|---|---|---|
| E/T | RD-012C202210001-NT | | | RD-012C202210001 | | |
| 3:1 | 13.27168 | 2.824294 | 2.574356 | 99.02524 | 99.27518 | 98.95026 |
| 1:1 | 4.698825 | −4.72382 | −5.39865 | 98.70032 | 99.05024 | 98.87528 |
| 1:3 | 16.04599 | 0.849788 | 2.774306 | 90.55236 | 91.72707 | 91.82704 |

E/T = Effector to target ratio

TABLE 3
Specific values of cytokine secretion level after
BCMA-CD19 CAR-T cells killed primary B cells (5 h)

| | Group | | |
|---|---|---|---|
| E/T | RD-012C202210001-NT | RD-012C202210001 | B cells only |

IFN-γ (ng/mL)

| | | | | | | | | | |
|---|---|---|---|---|---|---|---|---|---|
| 1:1 | 0 | 0 | 0 | 2962.35 | 2955.57 | 2996.34 | 0 | 0 | 0 |
| 1:3 | 0 | 0 | 0 | 2000.25 | 2186.64 | 2013.03 | 0 | 0 | 0 |
| 1:9 | 0 | 0 | 0 | 1161.54 | 1325.73 | 1222.17 | 0 | 0 | 0 |

IL-2 (ng/mL)

| | | | | | | | | | |
|---|---|---|---|---|---|---|---|---|---|
| 3:1 | 127.65 | 117.84 | 116.43 | 309.12 | 335.31 | 378.63 | 0 | 0 | 0 |
| 1:1 | 37.98 | 36.6 | 40.08 | 176.82 | 178.23 | 176.82 | 0 | 0 | 0 |
| 1:3 | 0 | 0 | 0 | 109.65 | 116.64 | 118.05 | 0 | 0 | 0 |

TNF-α (ng/mL)

| | | | | | | | | | |
|---|---|---|---|---|---|---|---|---|---|
| 3:1 | 0 | 0 | 0 | 0 | 283.38 | 289.53 | 0 | 0 | 0 |
| 1:1 | 0 | 0 | 0 | 0 | 0 | 255.84 | 0 | 0 | 0 |
| 1:3 | 0 | 0 | 0 | 0 | 0 | 0 | 0 | 0 | 0 |

IL-10 (ng/mL)

| | | | | | | | | | |
|---|---|---|---|---|---|---|---|---|---|
| 3:1 | 13.62 | 15.27 | 13.62 | 173.04 | 173.04 | 182.76 | 0 | 0 | 0 |
| 1:1 | 0 | 0 | 0 | 74.73 | 86.88 | 82.2 | 0 | 0 | 0 |
| 1:3 | 0 | 0 | 0 | 21.24 | 36.21 | 29.97 | 0 | 0 | 0 |

IL-4 (ng/mL)

| | | | | | | | | | |
|---|---|---|---|---|---|---|---|---|---|
| 3:1 | 0 | 0 | 0 | 21.06 | 22.35 | 21.06 | 0 | 0 | 0 |
| 1:1 | 0 | 0 | 0 | 1.05 | 9.54 | 5.61 | 0 | 0 | 0 |
| 1:3 | 0 | 0 | 0 | 10.83 | 5.61 | 8.25 | 0 | 0 | 0 |

IL-6 (ng/mL)

| | | | | | | | | | |
|---|---|---|---|---|---|---|---|---|---|
| 3:1 | 0 | 0 | 0 | 0 | 0 | 0 | 0 | 0 | 0 |
| 1:1 | 0 | 0 | 0 | 0 | 0 | 0 | 0 | 0 | 0 |
| 1:3 | 0 | 0 | 0 | 0 | 0 | 0 | 0 | 0 | 0 |

E/T = Effector to target ratio

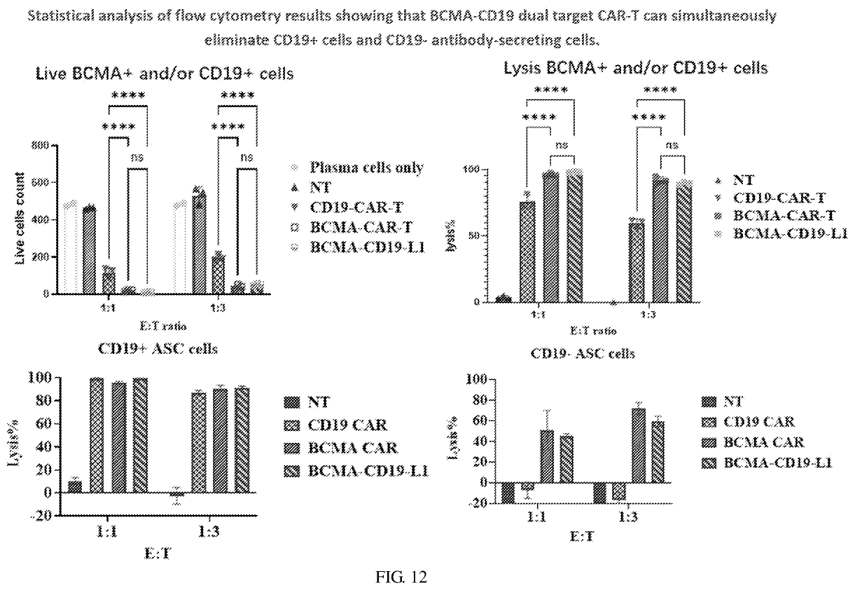

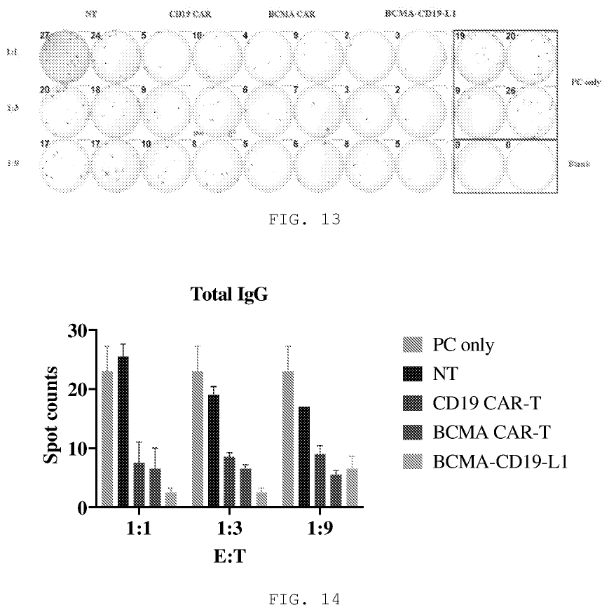

Table 3 and FIGS. 12-16 show the cytokine secretion level after BCMA-CD19 dual-target CAR-T cells killed primary B cells (5 h).

The results of cytokine release showed that a certain amount of IFN-r related to the killing efficiency of CAR-T cells and a small amount of cytokine IL-2 related to proliferation were indeed released during the killing process, and other factors were released less. This release of cytokines was similar to that of killing tumor cells in the past, suggesting that the cytokine secretion pattern may not be quite different from the existing CRS data in human body when it is used to treat SLE, which has certain implications for its safety.

TABLE 4
Specific values of BCMA-CD19CAR-T cells
killing antibody-secreting cells (5 h)

| | Group | |
|---|---|---|
| E/T | RD-012C202210001-NT | RD-012C202210001 |

CD19+ and/or BCMA+ cells

| | | | | | | |
|---|---|---|---|---|---|---|
| 1:1 | −11.9 | 2.4 | −14.7 | 95 | 96.6 | 95.8 |
| 1:3 | 4.3 | −1.1 | −2.8 | 87 | 87.2 | 85.5 |
| 1:9 | −2.7 | 0.6 | 1.2 | 52.7 | 53.6 | 52.7 |

Q1: CD19− BCMA+ cells

| | | | | | | |
|---|---|---|---|---|---|---|
| 1:1 | −67.6 | −5.8 | −26.8 | 88.9 | 93.6 | 93 |
| 1:3 | 11.9 | 0.2 | −4 | 79.9 | 76.7 | 80.5 |
| 1:9 | −0.4 | 2.6 | 8.7 | 31.2 | 33 | 30 |

Q2: CD19+ BCMA+ cells

| | | | | | | |
|---|---|---|---|---|---|---|
| 1:1 | 0.4 | 5.9 | −13.5 | 97.7 | 98.6 | 97.4 |
| 1:3 | 13.5 | 4.6 | −3.4 | 88.8 | 89.9 | 86 |
| 1:9 | 3.1 | 1.8 | −5.1 | 55.4 | 57.5 | 56.1 |

Q3: CD19+ BCMA− cells

| | | | | | | |
|---|---|---|---|---|---|---|
| 1:1 | 11.1 | 3.6 | −7.8 | 95.9 | 96.2 | 95.6 |
| 1:3 | −12.3 | −9 | −1.2 | 89.5 | 91 | 88.4 |
| 1:9 | −11.4 | −2.3 | 4 | 64.2 | 62.9 | 64.1 |

E/T = Effector to target ratio

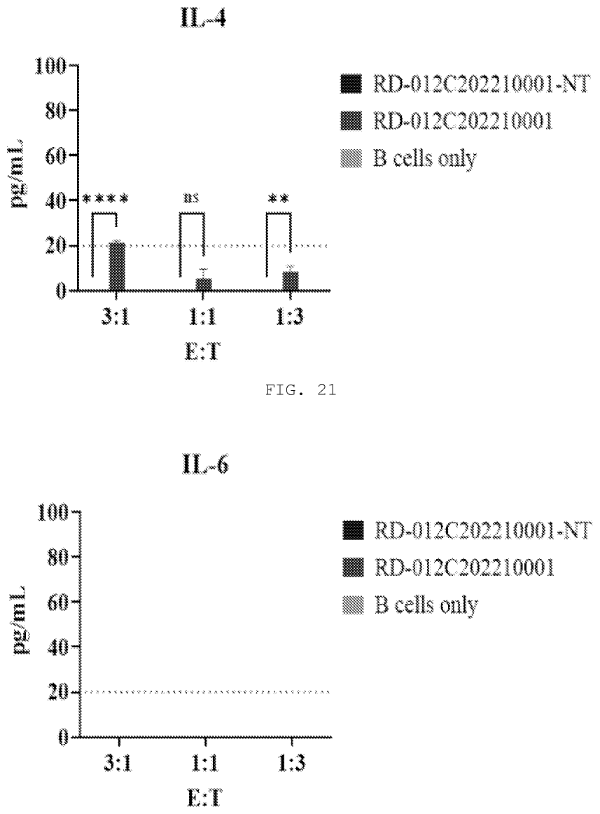

Table 4 and FIG. 19 show the situation after killing of antibody secreting cells by BCMA-CD19 dual-target CAR-T cells (5 h).

TABLE 5
Specific values of cytokine secretion level after killing
of antibody secreting cells by BCMA-CD19 CAR-T cells (5 h)

| | Group | | |
|---|---|---|---|
| E/T | RD-012C202210001-NT | RD-012C202210001 | ASC only |

IFN-γ (ng/mL)

| | | | | | | | | | |
|---|---|---|---|---|---|---|---|---|---|
| 3:1 | 134.05 | 0 | 0 | 5721.3 | 6497 | 5978.15 | 0 | 0 | 0 |
| 1:1 | 0 | 0 | 0 | 2904.65 | 2713.8 | 2666.2 | 0 | 0 | 0 |
| 1:3 | 0 | 0 | 0 | 927.6 | 880.9 | 880.9 | 0 | 0 | 0 |

IL-2 (ng/mL)

| | | | | | | | | | |
|---|---|---|---|---|---|---|---|---|---|
| 3:1 | 0 | 0 | 3.75 | 6.45 | 7.8 | 10.55 | 0 | 1 | 0 |
| 1:1 | 0 | 0 | 0 | 11.85 | 9.2 | 6.45 | 0 | 1 | 0 |
| 1:3 | 0 | 2.35 | 0 | 10.55 | 2.35 | 3.75 | 0 | 1 | 0 |

TNF-α (ng/mL)

| | | | | | | | | | |
|---|---|---|---|---|---|---|---|---|---|
| 3:1 | 2.55 | 2.55 | 2.55 | 27.15 | 91.15 | 131.35 | 0 | 0 | 0 |
| 1:1 | 0 | 5.85 | 0 | 44.35 | 58.3 | 47.15 | 0 | 0 | 0 |
| 1:3 | 0 | 9.05 | 2.55 | 18.25 | 9.05 | 21.25 | 0 | 0 | 0 |

IL-10 (ng/mL)

| | | | | | | | | | |
|---|---|---|---|---|---|---|---|---|---|
| 3:1 | 5.55 | 9.15 | 9.15 | 56.25 | 57.6 | 60.3 | 3.6 | 3.6 | 0 |
| 1:1 | 3.6 | 3.6 | 0 | 32.5 | 41.1 | 32.5 | 3.6 | 3.6 | 0 |
| 1:3 | 5.55 | 1.55 | 1.55 | 17.45 | 25.15 | 14.2 | 3.6 | 3.6 | 0 |

IL-4 (ng/mL)

| | | | | | | | | | |
|---|---|---|---|---|---|---|---|---|---|
| 3:1 | 0 | 0 | 0 | 23.75 | 23.75 | 30.6 | 0 | 0 | 0 |
| 1:1 | 0 | 1.55 | 0 | 14.7 | 14.7 | 14.7 | 0 | 0 | 0 |
| 1:3 | 0 | 0 | 1.55 | 5.85 | 5.85 | 5.85 | 0 | 0 | 0 |

IL-6 (ng/mL)

| | | | | | | | | | |
|---|---|---|---|---|---|---|---|---|---|
| 3:1 | 5.95 | 0 | 1.55 | 0 | 3.75 | 5.95 | 0 | 0 | 0 |
| 1:1 | 1.55 | 0 | 0 | 0 | 5.95 | 0 | 0 | 0 | 0 |
| 1:3 | 1.55 | 8.2 | 3.75 | 3.75 | 0 | 3.75 | 0 | 0 | 0 |

E/T = Effector to target ratio

Table 5 and FIG. 20 show the cytokine secretion level of BCMA-CD19 double target CAR-T cells after killing antibody secreting cells (5 h).

### Example 6: Detection of Cytokines in Supernatant

### Solution Preparation:

Preparation of mix beads: IL-2, IL-4, IL-6, IL-10, TNF-α and IFN-γ beads were added, each 808 μL, totaling 4.848 mL, and 19.392 mL of assay diluent was added with shaking, and the mixture was mixed evenly to obtain mix beads with a total volume of 24.24 mL;
Diluting the sample: The sample was diluted it with assay diluent at 1:1, and the total volume of the diluted sample was 50 μL/test;
Preparation of standard: 2 mL of assay diluent was added to the standard powder and left at room temperature for 15 min for use as Top standard; thereafter, the standard was diluted at 2× with assay diluent successively, and the last one was assay diluent;

### Operating Steps:

A 96-well U-bottom plate was taken, 50 μL of mix beads+50 μL of standards/samples+50 μL of Human TH1-TH2 PE Detection Reagent were added in turn, the plated was shaken for 5 min, and left in the dark at room temperature for 3 h;
100 μL of wash buffer was added, the system was mixed well, centrifuged at 300 g for 5 min, and the supernatant was discarded;
The beads were re-suspended with 100 μL of wash buffer;
The system was shaken for 2 min, loaded into the machine, with a sample size of 50 μL;

### Data Analysis

The determination coefficient R2 of linear equation of standard curve is ≥0.99; for the results of cytokine concentration, the cytokine concentration was analyzed by FCAP Array™ (BD).

### Example 7: Killing of B Cells in Peripheral Blood Samples of SLE Patients

The peripheral blood samples of SLE patients were taken and diluted with DPBS at 1:1 for later use;
The ficoll separation medium at room temperature was taken, the ficoll and the diluted peripheral blood sample were added at a volume ratio of 1:3;
The speed-up and speed-down of the centrifuge were adjusted to 4 and 3 respectively, the centrifugal speed was 1000 g, and the system was centrifuged at room temperature for 20 min;
The PBMC cells in the buffy coat were sucked out with a pipette, counted, centrifuged at 400 g for 8 min to collect the cells, which were the target cells.

The target cells were re-suspended in 1640 complete medium at a concentration of 5×10⁵ cells/mL;
According to the E:T ratio of the experimental plan, the effector T cells were diluted in gradient.

A clean 96-well U-bottom plate was taken, the target cells and effector cells were added respectively, each 100 μl, the plate was labeled and put in a 37° C. carbon dioxide incubator to incubate for 5 h;
After incubation, the supernatant was taken as a reserved sample;
The remaining cells were centrifuged and FACS buffer was added: each 100 μl of FACS buffer comprises 1 μl of each of the following antibodies: APC-anti-CD38, BV421-anti-CD138, BUV395-anti-CD27, BV510-anti-CD45, FITC-anti-CD3/CD14, Pe-Cy7-antiCD19, BV786-anti-IgD. After mixing, the system was stained at 4° C. for 30 min in the dark;
It was washed twice with FACS buffer, and the third time, it was re-suspended with 1:4800 diluted DAPI FACS buffer and loaded on the machine for detection. Samples with equal absolute volume were collected from each well to analyze the absolute number of living B cells and antibody-secreting cells in the residual samples. The results are shown in FIGS. 21-24; in FIG. 21, non-ASC refers to non-antibody-secreting cells, ASC refers to antibody-secreting cells that are CD38 and CD27 positive, CD138-positive ASC refers to plasma cells (PCs), and CD19-PC refers to CD19-negative plasma cells. Subsequent killing experiments are all analyzed based on the above gating.

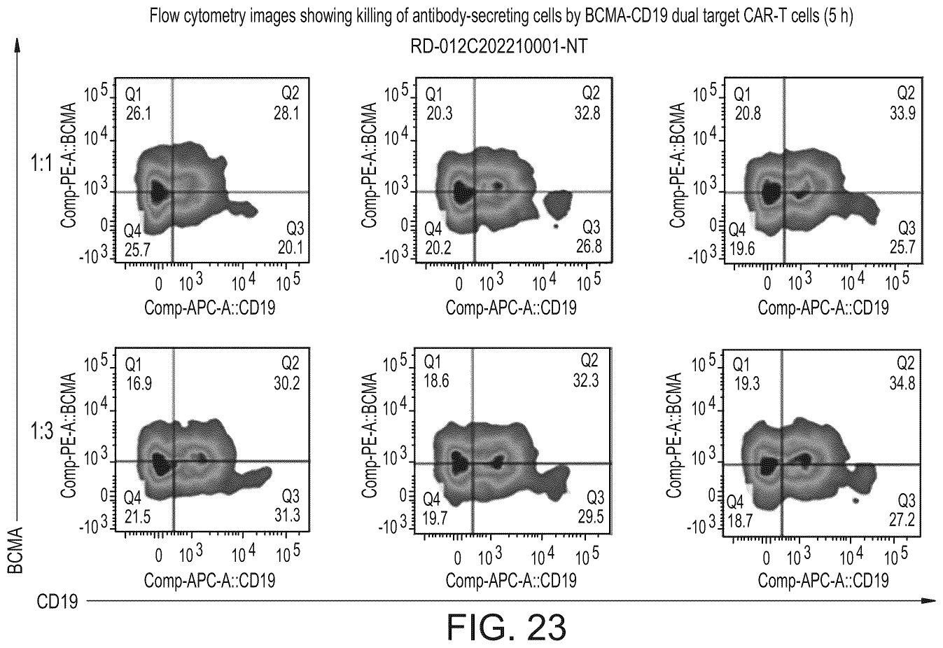

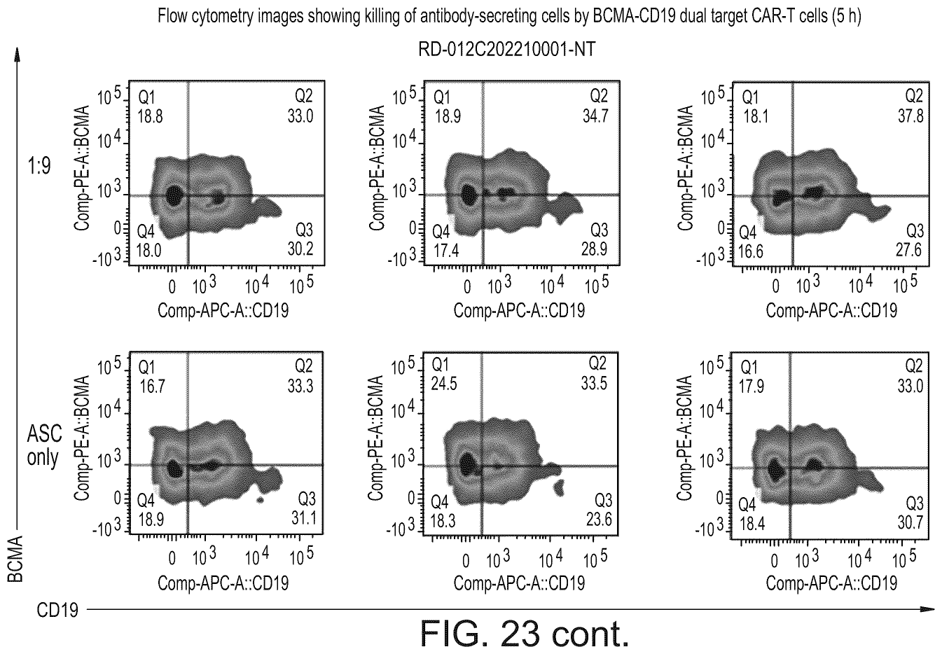

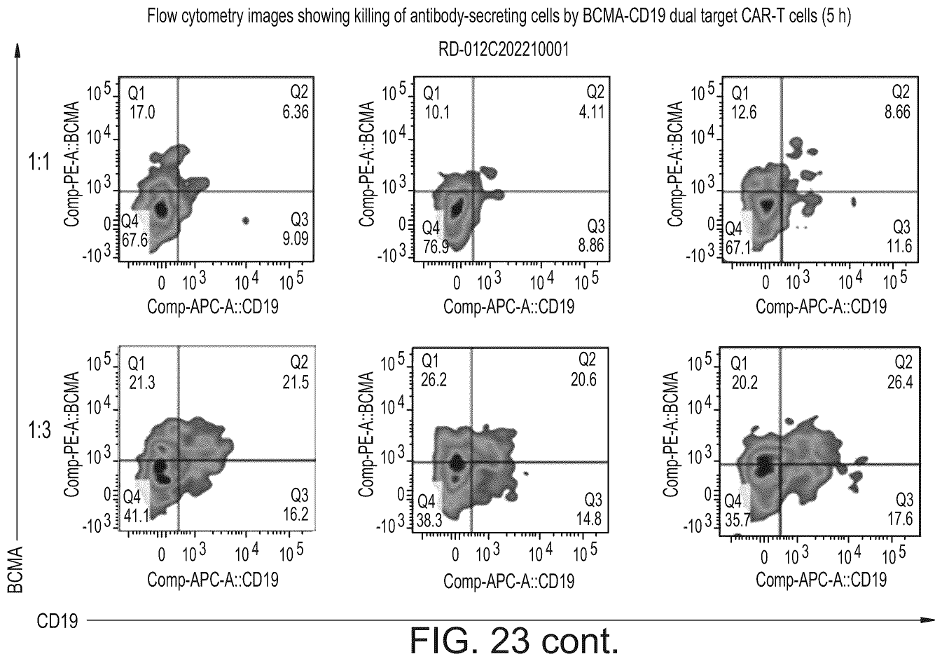

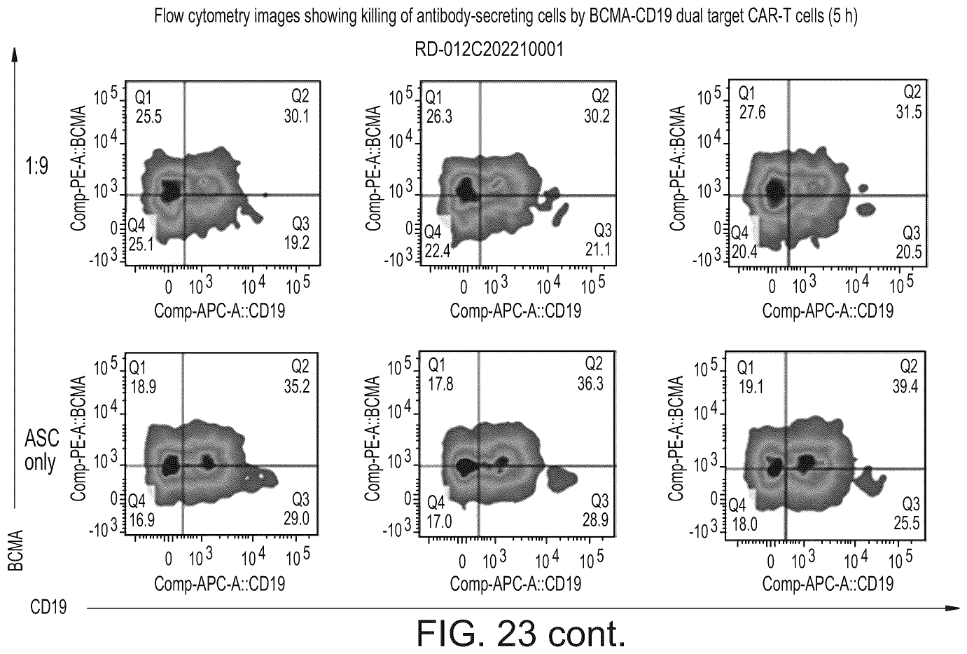

The SLE samples were co-incubated with BCMA-CD19 dual-target CAR-T and control NT cells for 5 h according to a certain ratio, and then the absolute number and percentage changes of B cells, antibody-secreting cells and plasma cells in the samples after incubation were detected. The flow cytometry images and statistical absolute number of B cells are shown in FIG. 23. The change of B cells in SLE samples and the statistics of absolute number of residual cells after the killing are shown in FIG. 23. It can be seen that BCMA-CD19 dual-target CAR-T can significantly kill B cells in SLE samples.

The flow cytometry images and statistical absolute number of ASC cells are shown in FIG. 23. The change of antibody-secreting cells in SLE samples and the statistical absolute number of residual cells after the killing are shown in FIG. 23B. It can be seen that BCMA-CD19 dual-target CAR-T can significantly kill antibody-secreting cells in SLE samples.

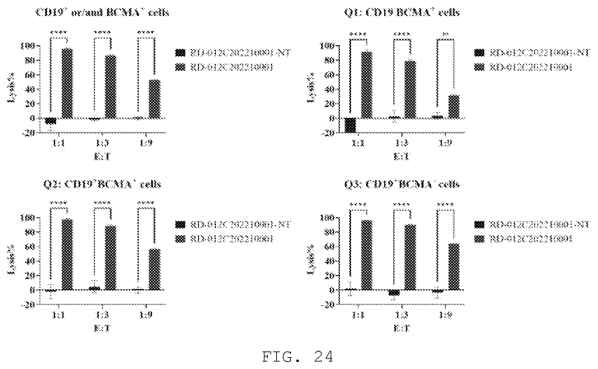

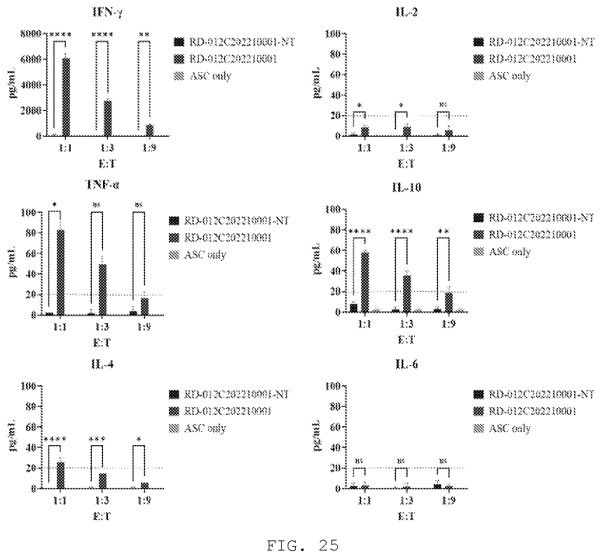

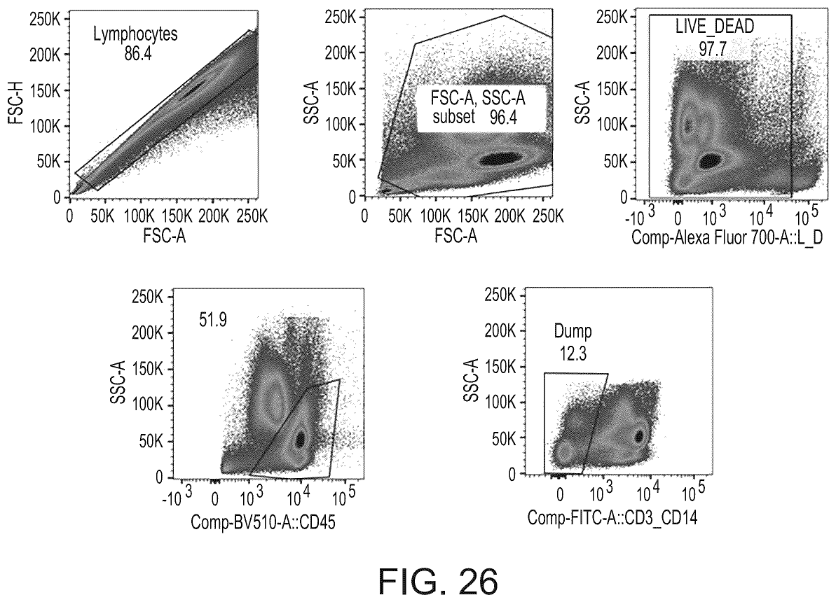

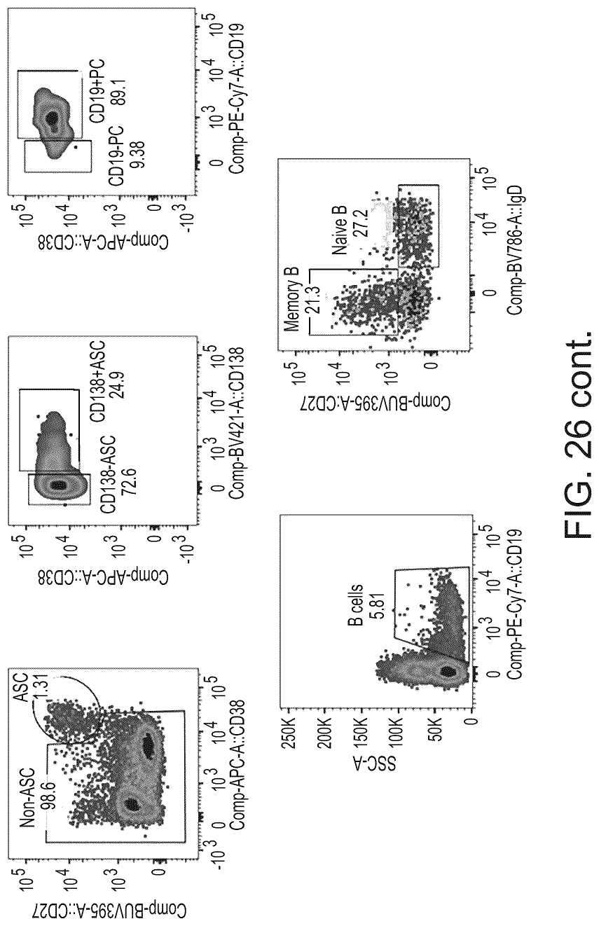

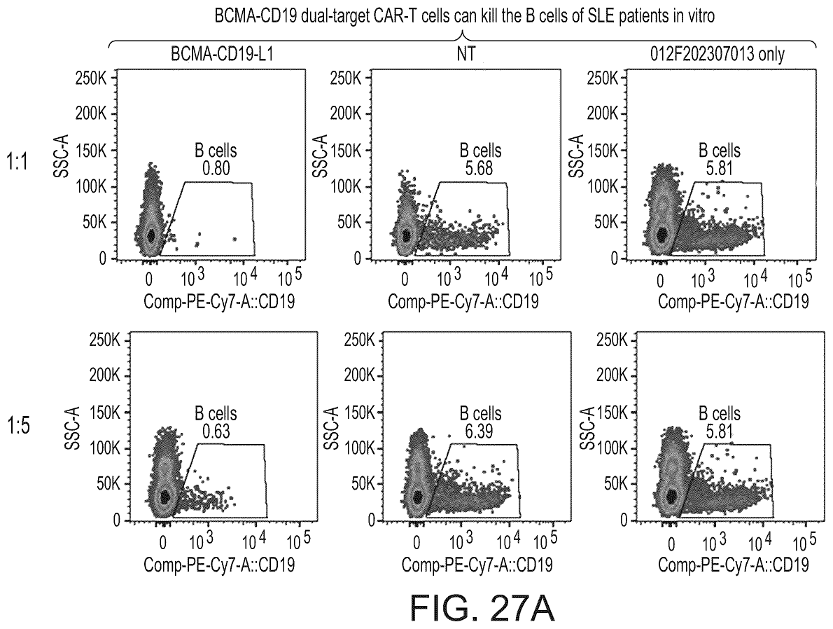

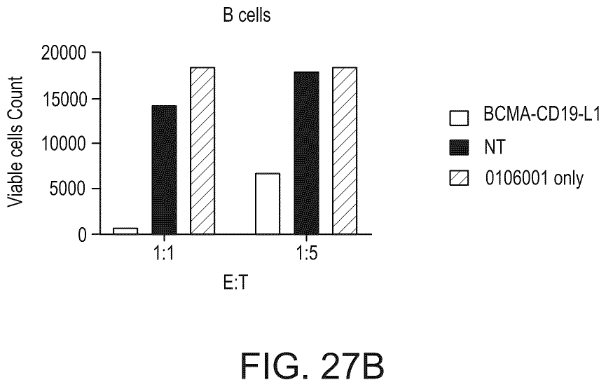

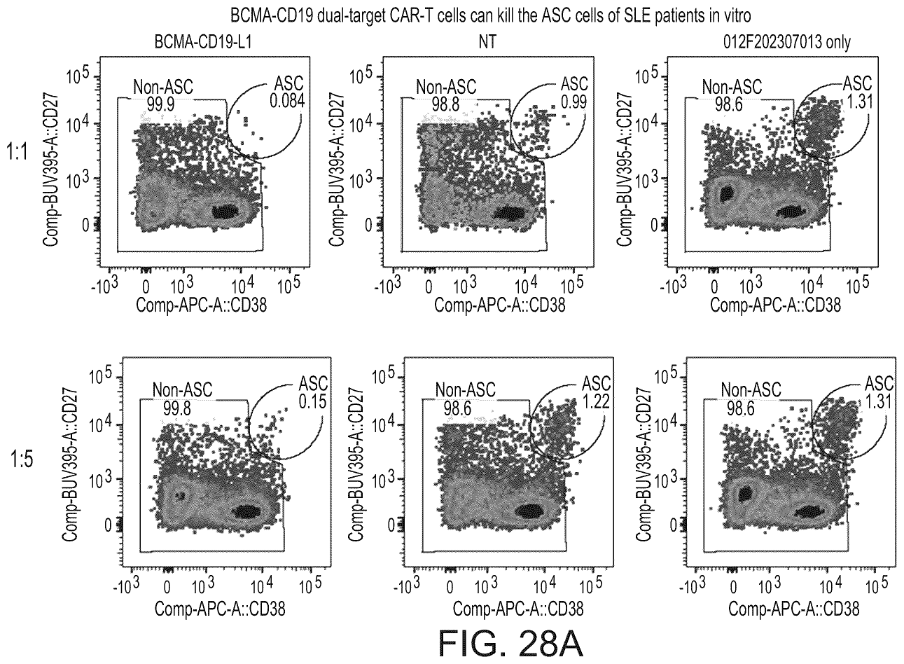

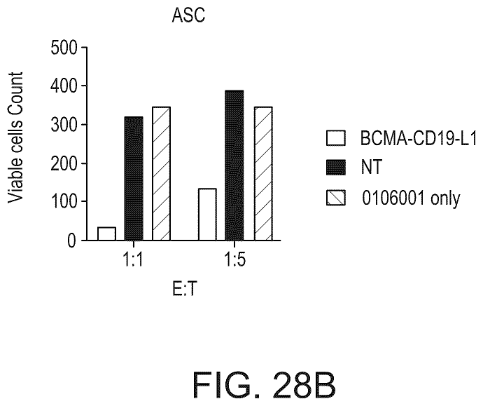

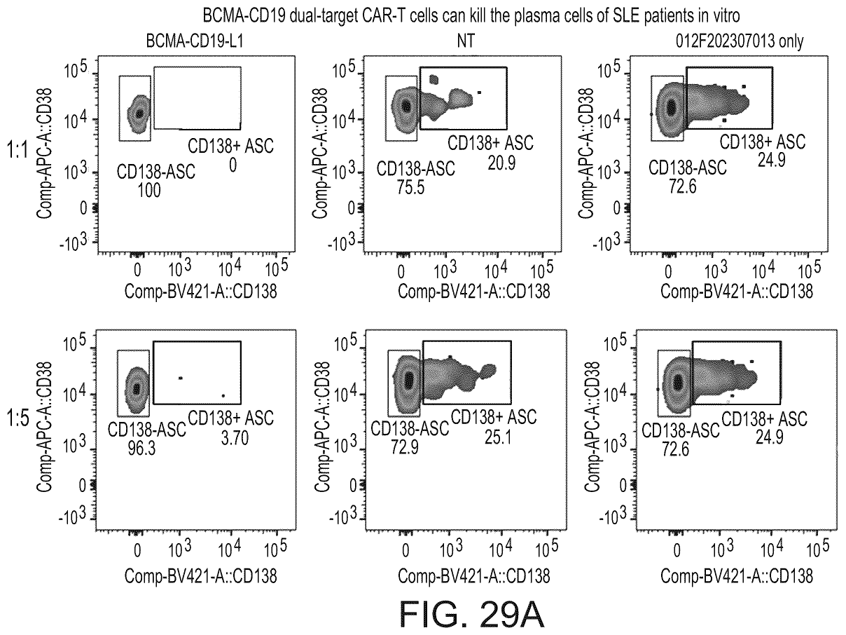

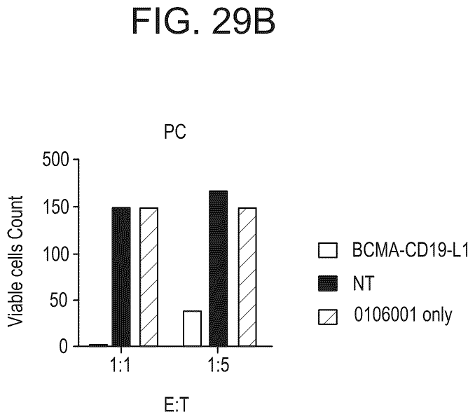

The flow cytometry images and statistical absolute number of plasma cells are shown in FIG. 24. The changes of plasma cells in SLE samples and the statistical absolute number of residual cells are shown in FIG. 24 below. It can be seen that BCMA-CD19 dual-target CAR-T can significantly kill plasma cells in SLE samples.

The results in FIGS. 22-24 show that BCMA-CD19 CAR-T cells can kill B cells and ASC cells (including CD138+ plasma cells) of SLE patients in vitro.

So far, those skilled in the art should realize that although several exemplary embodiments of the disclosure have been shown and described in detail herein, many other variations or modifications in line with the principles of the disclosure can be directly determined or deduced from the disclosure without departing from the spirit and scope of the disclosure. Therefore, the scope of the disclosure should be understood and deemed to cover all such other variations or modifications.

### Example 8: BCMA and CD19 Antigen Density in B Cell Subsets Across Sjogren's Syndrome, Scleroderma or Systemic Sclerosis (SSc), Myositis, IgA Nephropathy, and Healthy Donors

Examples 8-13 used a bispecific CAR having a loop structure (e.g., formula 1a)). The ability to deplete BCMA+ cells and CD19+ cells is dependent on antigen density on the cell surface. Fresh or frozen peripheral blood mononuclear cells (PBMCs) from donors having Sjogren's syndrome, scleroderma or systemic sclerosis (SSc), or myositis or IgA nephropathy as well as healthy donors were assessed for antigen density of CD19 and BCMA.

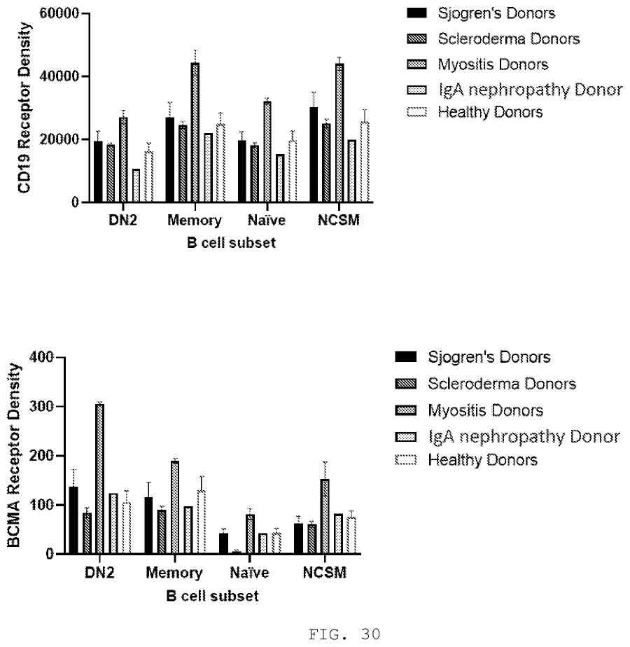

As seen in FIG. 30, target expression of CD19 and BCMA is broadly similar across B cell subsets and disease indication, excepting the elevation seen in myositis patients.

### Example 9: Representative Gating Strategy for In Vitro BCMA-CD19 CAR-T Cell Killing Assay

Total PMBCs Sjogren's syndrome, scleroderma or systemic sclerosis (SSc), myositis, IgA nephropathy, and healthy donors were counted and plated for flow cytometry staining. After thawing, cells were stained with following antibodies (Live/Dead stain, CD20, dump gate—CD3, CD14, CD16, and CD56, CD19, CD27, CD38, IgD, CD138). All cells shown in FIG. 30 are lymphocytes defined by forward scatter/side scatter (FSC/SSC), live cells, dump gate negative, CD20+/−, and CD19+/low. DN2 cells (IgD−, CD27−), Memory B cells (CD27+IgD−), Naïve B cells (IgD+CD27−), NCSM (non-class switched memory, IgD+CD27).

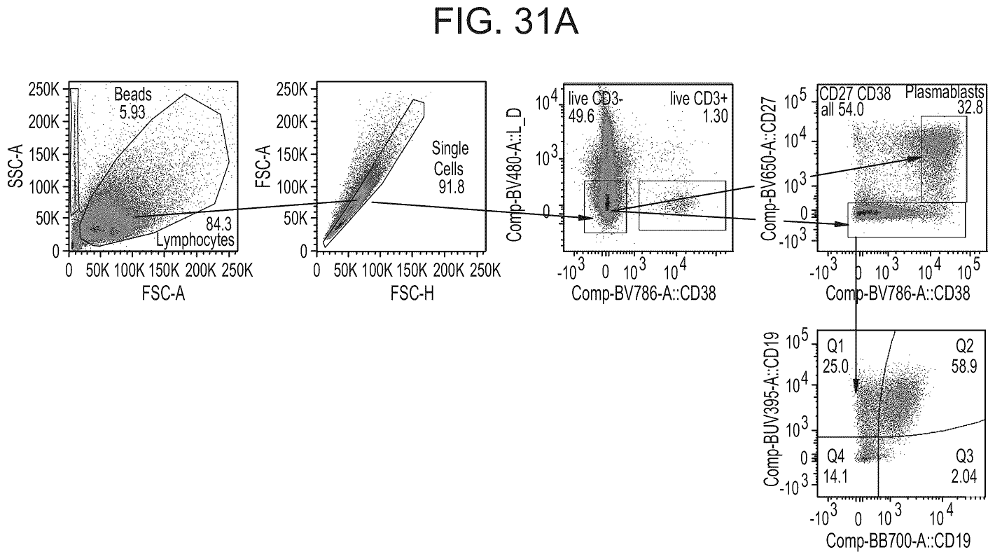

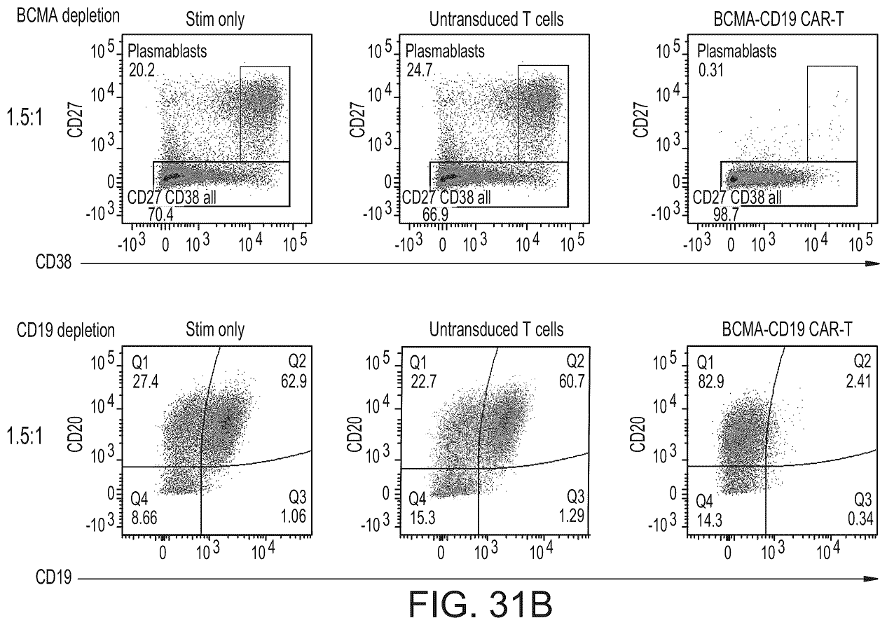

The gating strategy shown in FIG. 31A shows how the target cells for both arms of the CAR-T cell were identified for percent depletion calculation following co-culture with BCMA-CD19 CAR-T cells. In vitro killing of BCMA+ and CD19+ targets is shown in FIG. 31B.

### Example 10: BCMA-CD19 CAR-T Cells Deplete In Vitro Differentiated BCMA+ Targets and CD19 Targets from Healthy Donors

BCMA-CD19 CAR-T cell research lots were manufactured from fresh peripheral blood of individual healthy donors participating in the AstraZeneca blood donation program. Briefly, healthy donors' PBMCs were harvested from blood by Ficoll gradient centrifugation, and CD4 and CD8 positive T cells were enriched from the leukocyte fraction. Isolated T cells were then activated, transduced with BCMA-CD19 BZ lentiviral vector, and expanded in culture flasks before cell were washed, harvested and frozen in a cryopreservation medium.

Naïve B-cells from frozen healthy donor PBMCs were isolated with Naïve B-cell isolation kit (Stemcell). After isolation, naïve B-cells were plated and differentiated utilizing a proprietary mix of cytokines to drive B-cell differentiation. After 5 days of differentiation, the differentiated B-cells were co-cultured with BCMA-CD19 CAR-T cells or untransduced T cells at 1.5:1, 0.75:1, 0.37:1, 0.18:1, 0.093:1, and 0.0461:1 effector:target ratios for 24 hours. Co-cultured cells were then stained with the following reagents: (Live/Dead stain, CD20, CD19, CD27, CD38, BCMA, CD3, CD4, CD8, CD69). Killing of differentiated plasma blasts (live, CD3−, CD27+ CD38+) and CD19+ targets (live CD3−, CD27−, CD38+/−, CD19+CD20+) was assessed by flow cytometry. Percent depletion was calculated by 1-(% targets in experimental well/% average targets in stim only wells)*100.

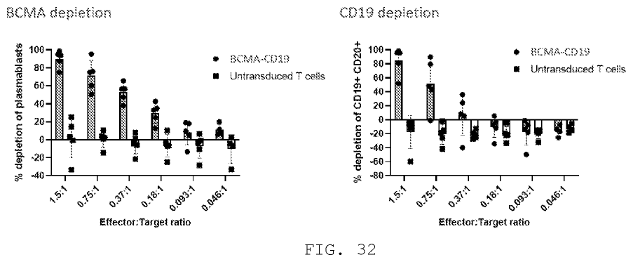

The bispecific CAR was able to efficiently deplete BCMA+ and CD19+ targets in healthy controls as shown in FIG. 32. A dose dependent depletion (E:T) by BCMA-CD19 CAR-T cells is observed.

### Example 11

Co-Culture of BCMA-CD19 CAR-T Cells with In Vitro Differentiated BCMA+ and CD19+ Targets Derived from Healthy Donor PBMCs Drives a Dose Dependent Secretion of Cytokines Associated with T Cell Activation.

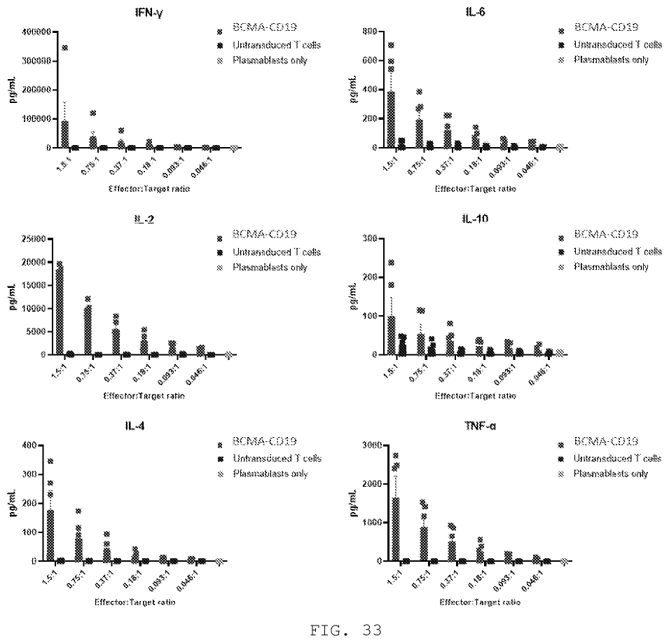

After 24 hour of co-culture of differentiated B cells and BCMA-CD19 CAR-T cells supernatants were collected, aliquoted and stored at −80° C. for later Meso Scale Discover (MSD) analysis. The BCMA-CD19 CAR-T cells demonstrated dose dependent secretion of IFNγ, IL-2, IL-4, IL-6, IL-10 and TNFα in response to healthy donor derived targets as seen in FIG. 33. This shows target specific activation of the CAR T cells.

### Example 12

BCMA-CD19 CAR-T cells deplete in vitro differentiated BCMA+ targets and CD19 targets from Sjogren's Syndrome, scleroderma or systemic sclerosis (SSc), myositis donors, and IgA nephropathy donors.

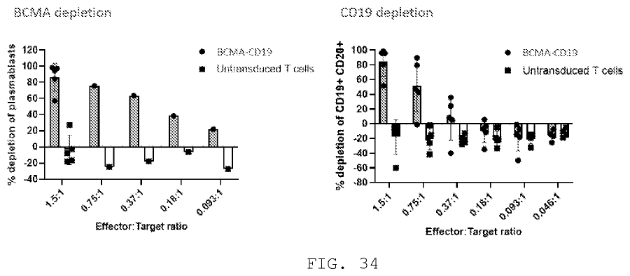

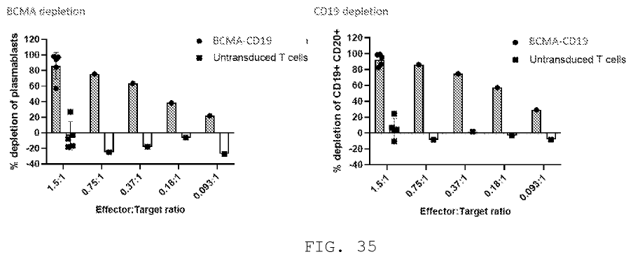

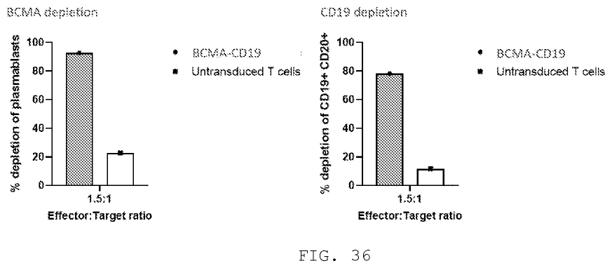

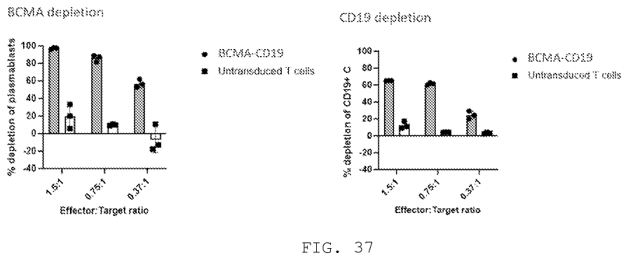

Primary human naïve B-cells (IgD+CD27− selection) were isolated from frozen Sjogren's Syndrome, scleroderma or systemic sclerosis (SSc), myositis and patient PBMCs. The naïve B-cells were then differentiated using a proprietary mixture of cytokines to drive plasmablast differentiation (BCMA+ B-cells). After 5 days of differentiation, the cells were then co-cultured with the BCMA-CD19 Bz CAR T cells (manufactured with preclinical manufacturing process) or un-transduced T cells (untargeted) at varying effector:target ratios. The BCMA-CD19 CAR-T was able to deplete BCMA+ and CD19+ targets to similar extent in Sjogren's Syndrome (FIG. 34), scleroderma or systemic sclerosis (FIG. 35) myositis (FIG. 36) and IgA nephropathy (FIG. 37) as seen in healthy controls.

### Example 13

Co-culture of BCMA-CD19 CAR-T cells with in vitro differentiated BCMA+ and CD19+ targets derived from Sjogren's syndrome, Scleroderma, Myositis and IgA nephropathy donors drives a dose dependent secretion of cytokines associated with T cell activation to similar extent.

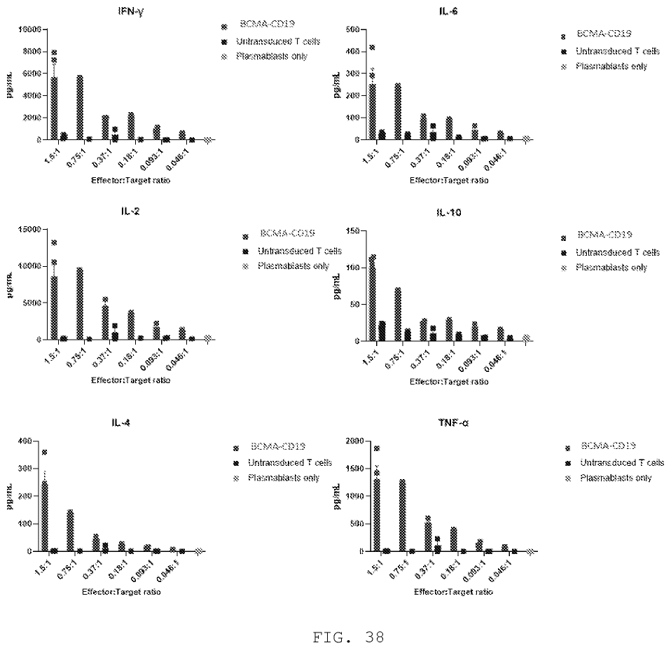

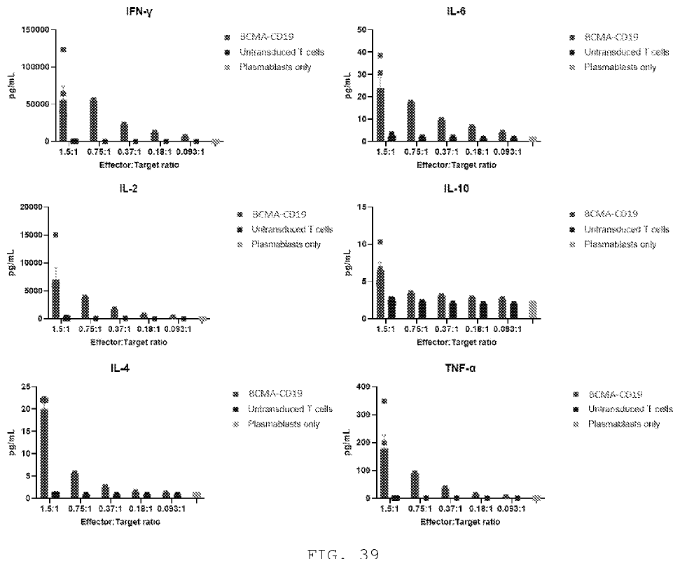

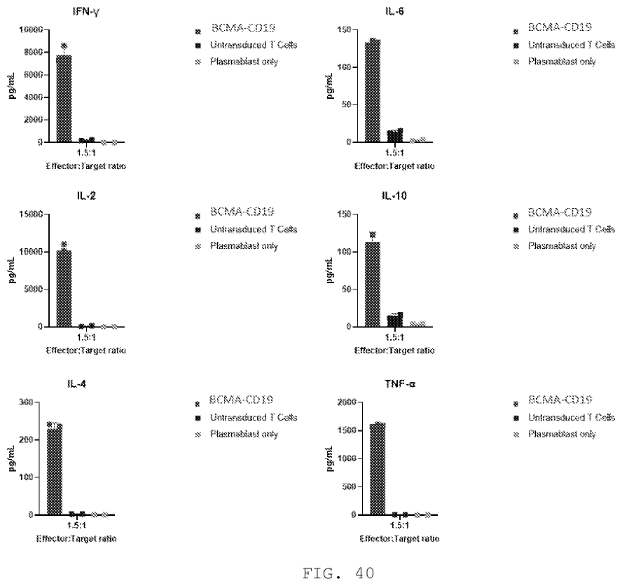

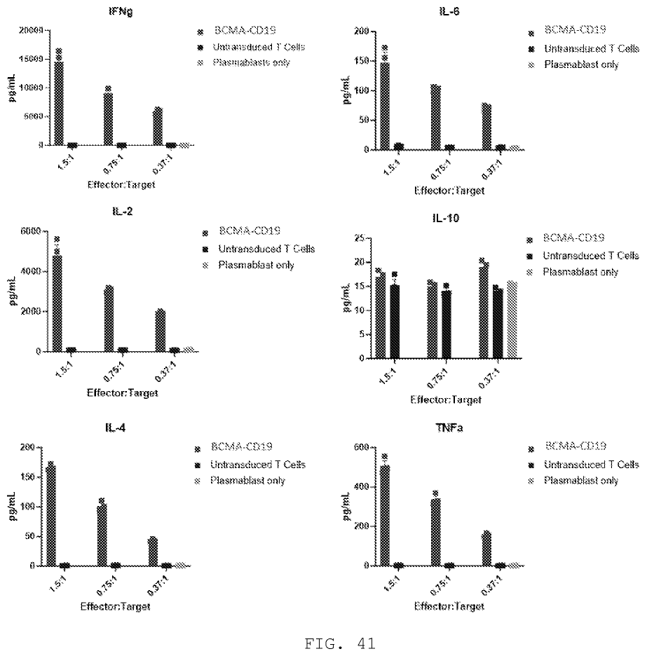

After 24-hour co-culture of differentiated B cells and BCMA-CD19 CAR-T cells supernatants were collected, aliquoted and stored at −80° C. for later MSD analysis. The BCMA-CD19 CAR-T cells demonstrated dose dependent secretion of IFNγ, IL-2, IL-4, IL-6, IL-10 and TNFα in response to Sjogren's syndrome (FIG. 38), scleroderma or systemic sclerosis (FIG. 39), myositis (FIG. 40) and IgA nephropathy (FIG. 41) derived targets.

The invention claimed is:

1. A method of treating and/or preventing an autoimmune disease comprising administering a bispecific chimeric antigen receptor (CAR), wherein a first target of the CAR is CD19, and a second target of the CAR is BCMA.

2. The method according to claim 1, wherein the CAR is in combination with one or more of the following agents:
(1) an agent that increases the efficacy of cells comprising CAR nucleic acids or CAR polypeptides;
(2) an agent for improving one or more side effects associated with the administration of cells comprising CAR nucleic acids or CAR polypeptides; and/or
(3) an additional agent for treating diseases related to BCMA and CD19.

3. The method according to claim 1, wherein an antibody targeting BCMA or an antigen-binding fragment thereof or an antibody targeting CD19 or an antigen-binding fragment thereof is independently selected from camel Ig, IgNAR, Fab fragment, Fab′ fragment, F(ab′)z fragment, F(ab′)3 fragment, Fv, single-chain antibody including scFv, di-scFv, and (scFv)z, micro-antibody, bifunctional antibody, trifunctional antibody, tetrafunctional antibody, disulfide bond-stabilized Fv protein (“dsFv”) and single-domain antibody (sdAb, nano-antibody), chimeric antibody, humanized antibody, single-domain antibody, bispecific antibody or multispecific antibody, binding ligand, or protein domain.

4. The method according to claim 1, wherein the antigen-binding fragment targeting BCMA or the antigen-binding fragment targeting CD19 is scFv.

5. The method according to claim 1, wherein the antibody or antigen-binding fragment thereof targeting BCMA comprises a heavy chain variable region (VH,BCMA) comprising a complementarity determining region 1 (HCDR1) comprising the amino acid sequence of SEQ ID NO: 5, a HCDR2 comprising the amino acid sequence of SEQ ID NO: 6, and a HCDR3 comprising the amino acid sequence of SEQ ID NO: 7; and
a light chain variable region (VL, BCMA) comprising a complementarity determining region 1 (LCDR1) comprising the amino acid sequence of SEQ ID NO: 8, a LCDR2 comprising the amino acid sequence of SEQ ID NO: 9, and a LCDR3 comprising the amino acid sequence of SEQ ID NO: 10.

6. The method according to claim 1, wherein the antibody or antigen-binding fragment targeting CD19 comprises a heavy chain variable region (VH,CD19) comprising a complementarity determining region 1 (HCDR1) comprising the amino acid sequence of SEQ ID NO: 11, a HCDR2 comprising the amino acid sequence of SEQ ID NO: 12, and a HCDR3 comprising the amino acid sequence of SEQ ID NO: 13; and
a light chain variable region (VL,CD19) comprising a complementarity determining region 1 (LCDR1) comprising the amino acid sequence of SEQ ID NO: 14, a LCDR2 comprising the amino acid sequence of SEQ ID NO: 15, and a LCDR3 comprising the amino acid sequence of SEQ ID NO: 16.

7. The method according to claim 1, wherein
the antibody or antigen-binding fragment thereof targeting BCMA comprises
a heavy chain variable region (VH,BCMA) comprising a complementarity determining region 1 (HCDR1) comprising the amino acid sequence of SEQ ID NO: 5, a HCDR2 comprising the amino acid sequence of SEQ ID NO: 6, and a HCDR3 comprising the amino acid sequence of SEQ ID NO: 7;
a light chain variable region (VL,BCMA) comprising a complementarity determining region 1 (LCDR1) comprising the amino acid sequence of SEQ ID NO: 8, a LCDR2 comprising the amino acid sequence of SEQ ID NO: 9, and a LCDR3 comprising the amino acid sequence of SEQ ID NO: 10; and
wherein the antibody or antigen-binding fragment targeting CD19 comprises
a heavy chain variable region (VH,CD19) comprising a complementarity determining region 1 (HCDR1) comprising the amino acid sequence of SEQ ID NO: 11, a HCDR2 comprising the amino acid sequence of SEQ ID NO: 12, and a HCDR3 comprising the amino acid sequence of SEQ ID NO: 13; and
a light chain variable region (VL,CD19) comprising a complementarity determining region 1 (LCDR1) comprising the amino acid sequence of SEQ ID NO: 14, a LCDR2 comprising the amino acid sequence of SEQ ID NO: 15, and a LCDR3 comprising the amino acid sequence of SEQ ID NO: 16.

8-13. (canceled)

14. The method according to claim 7, wherein the VH,BCMA comprises the amino acid sequence of SEQ ID NO: 1; and the VL,BCMA comprises the amino acid sequence of SEQ ID NO: 2.

15. The method according to claim 7, wherein the VH,CD19 comprises the amino acid sequence of SEQ ID NO: 3; and the VL,CD19 comprises the amino acid sequence of SEQ ID NO: 4.

16. The method according to claim 1, wherein the structure of the bispecific CAR is selected from any one of the following formulas:
1a) L-VL,CD19-VH,BCMA-VL,BCMA-VH,CD19-H-TM-C-CD3ζ;
1b) L-VL,CD19-VL,BCMA-VH,BCMA-VH,CD19-H-TM-C-CD3ζ;
2a) L-VH,CD19-VL,BCMA-VH,BCMA-VL,CD19-H-TM-C-CD3ζ;
2b) L-VH,CD19-VH,BCMA-VL,BCMA-VL,CD19-H-TM-C-CD3ζ;
3a) L-VL,CD19-VH,CD19-H-TM-C-CD3ζ-2A peptide-L-VH,BCMA-VL,BCMA-H-TM′-C-CD3ζ;
3b) L-VH,CD19-VL,CD19-H-TM-C-CD3ζ-2A peptide-L-VH,BCMA-VL,BCMA-H-TM′-C-CD3ζ;
3c) L-VL,CD19-VH,CD19-H-TM-C-CD3ζ-2A peptide-L-VL,BCMA-VH,BCMA-H-TM′-C-CD3ζ;
3d) L-VH,CD19-VL,CD19-H-TM-C-CD3ζ-2A peptide-L-VL,BCMA-VH,BCMA-H-TM′-C-CD3ζ;
4a) L-VH,BCMA-VL,BCMA-H-TM-C-CD3ζ-2A peptide-L-VL,CD19-VH,CD19-H-TM′-C-CD3ζ;
4b) L-VL,BCMA-VH,BCMA-H-TM-C-CD3ζ-2A peptide-L-VL,CD19-VH,CD19-H-TM′-C-CD3ζ;
4c) L-VH,BCMA-VL,BCMA-H-TM-C-CD3ζ-2A peptide-L-VH,CD19-VL,CD19-H-TM′-C-CD3ζ;
4d) L-VL,BCMA-VH,BCMA-H-TM-C-CD3ζ-2A peptide-L-VH,CD19-VL,CD19-H-TM′-C-CD3ζ;
wherein:
each of 1a), 1b), 2a), and 2b) comprises a loop structure formed by the antibody or antigen binding fragments targeting BCMA and CD19;
“−” is independently a linker peptide or peptide bond;
L is absent or is a signal peptide sequence;
VH,BCMA is an anti-BCMA antibody heavy chain variable region, and VL,BCMA is an anti-BCMA antibody light chain variable region;
VH,CD19 is an anti-CD19 antibody heavy chain variable region, and VL,CD19 is an anti-CD19 antibody light chain variable region;
H is a hinge region;
TM is the first transmembrane domain;
TM′ is the second transmembrane domain;
2A peptide is selected from P2A, T2A, E2A or F2A;
C is a costimulatory signal molecule; and
CD3ζ is a cytoplasmic signal transduction sequence derived from CD3ζ.

17. The method according to claim 16, wherein L comprises a domain from one or more of CD8, CD28, GM-CSF, CD4, and CD137.

18. The method according to claim 16, wherein L comprises a domain from CD8.

19. The method according to claim 16, wherein L comprises the amino acid sequence of SEQ ID NO: 25.

20. The method according to claim 16, wherein H comprises a domain from one or more of CD8, CD28, and CD137.

21. The method according to claim 16, wherein H comprises one of the amino acid sequences of SEQ ID NOs: 17 to 19.

22. The method according to claim 21, wherein H comprises the amino acid sequence of SEQ ID NO: 19.

23. The method according to claim 16, wherein TM or TM′ comprises a domain from one or more of CD28, CD3 epsilon, CD45, CD4, CD5, CD8, CD9, CD16, CD22, CD33, CD37, CD64, CD80, CD86, CD134, CD137, and CD154.

24. The method according to claim 16, wherein TM or TM′ comprises a domain from CD8.

25. The method according to claim 16, wherein TM or TM′ comprises the amino acid sequence of SEQ ID NO: 20.

26. The method according to claim 16, wherein C comprises a domain from one or more of OX40, CD2, CD7, CD27, CD28, CD30, CD40, CD70, CD134, 4-1BB (CD137), PD1, Dap10, CDS, ICAM-1, LFA-1(CD11a/CD18), ICOS (CD278), NKG2D, GITR, and TLR2.

27. The method according to claim 16, wherein C comprises a domain from one or more of 4-1BB.

28. The method according to claim 16, wherein C comprises the amino acid sequence of SEQ ID NO: 21.

29. The method according to claim 16, wherein CD3ζ comprises the amino acid sequence of SEQ ID NO: 22.

30. The method according to claim 16, wherein “−” in at least one instance comprises one or more of the amino acid sequence of SEQ ID NO: 23.

31. The method according to claim 16, wherein “−” in at least one instance comprises the amino acid sequence of SEQ ID NO: 24.

32. The method according to claim 16, wherein
H comprises the amino acid sequence of SEQ ID NO: 19;
L comprises the amino acid sequence of SEQ ID NO: 25;
VH,BCMA comprises the amino acid sequence of SEQ ID NO: 1;
VL,BCMA comprises the amino acid sequence of SEQ ID NO: 2;
VH,CD19 comprises the amino acid sequence of SEQ ID NO: 3;
VL,CD19 comprises the amino acid sequence of SEQ ID NO: 4;
TM comprises the amino acid sequence of SEQ ID NO: 20;
C comprises the amino acid sequence of SEQ ID NO: 21; and
CD3ζ comprises the amino acid sequence of SEQ ID NO: 22.

33. The method according to claim 32, wherein the bispecific CAR has the structure according to formula 1a).

34. A method for treating and/or preventing an autoimmune disease comprising administering a bispecific CAR comprising the amino acid sequence of SEQ ID NO: 26.

35. The method according to claim 34, wherein the bispecific CAR is administered in an amount of 0.5±20%×10\hat{ }5 cells/kg.

36. The method according to claim 34, wherein the bispecific CAR is administered in an amount of 1.020%×10\hat{ }5 cells/kg.

37. The method according to claim 34, wherein the bispecific CAR is administered in an amount of 2.020%×10\hat{ }5 cells/kg.

38. The method according to claim 34, wherein the bispecific CAR is administered in an amount of 3.020%×10\hat{ }5 cells/kg.

39. The method according to claim 34, wherein the bispecific CAR is administered in an amount of 0.5×10\hat{ }5 cells/kg, 1.0×10\hat{ }5 cells/kg, 2.0×10\hat{ }5 cells/kg, or 3.0×10\hat{ }5 cells/kg.

40. The method according to claim 34, wherein the treatment and/or prevention further comprises being used in combination with a second therapy selected from surgery, chemotherapy, radiotherapy, immunotherapy, gene therapy, DNA therapy, RNA therapy, nanotherapy, virus therapy, adjuvant therapy, and any combination thereof.

41. The method according to claim 34, wherein the bispecific CAR kills or inhibits CD19-positive B cells, plasmablasts and BCMA-positive plasma cells.

42. The method according to claim 34, wherein the autoimmune disease is a B-cell mediated autoimmune disease.

43. The method according to claim 42, wherein the B-cell mediated autoimmune disease includes one or more of systemic lupus erythematosus (SLE), glomerulonephritis includes autoimmune chronic kidney disease, lupus nephritis, acute glomerulonephritis, immune nephritis, membranous nephropathy, IgA nephropathy, Anti-Neutrophil Cytoplasmic Antibody (ANCA) Associated Vasculitis (AAV); scleroderma or systemic sclerosis (SSc); myositis or idiopathic inflammatory myositis including dermatomyositis, polymyositis, immune-mediated necrotizing myopathy (IMNM), antisynthetase syndrome, inclusion body myositis, and overlap myositis; multiple sclerosis (MS); inflammatory bowel disease (IBD); rheumatoid arthritis (RA); Sjogren's syndrome (SS); autoimmune hemolytic anemia; neuromyelitis optica (NMO); neuromyelitis optica spectrum disease (NMOSD); idiopathic thrombocytopenic purpura (ITP); systemic autoimmune small vessel vasculitis syndrome or polyangiitis related to antineutrophil cytoplasmic antibody; Wegener's granulomatosis (GPA), eosinophilic granulomatosis with polyangiitis (EGPA, Churg-Strauss syndrome); pemphigus vulgaris; autoimmune encephalitis; pemphigus vulgaris; myasthenia gravis; antiphospholipid syndrome; Chagas' disease; Graves' disease; polyarteritis nodosa; pulmonary hemorrhage-nephritis syndrome; Kawasaki disease, amyloidosis; monoclonal immunoglobulin of undetermined significance, POEMS syndrome; Crohn's disease; ulcerative colitis; adult onset Still's disease; and chronic progressive cortical demyelinating encephalopathy (CIDP).

44. The method according to claim 43, wherein the autoimmune disease is systemic lupus erythematosus; the systemic lupus erythematosus includes moderate to severe refractory systemic lupus erythematosus, lupus nephritis, active lupus nephritis and active systemic lupus erythematosus without renal involvement.

45. The method according to claim 43, wherein the autoimmune disease is myositis.

46. The method according to claim 43, wherein the autoimmune disease is glomerulonephritis.

47. The method according to claim 46, wherein the glomerulonephritis is at least one of autoimmune chronic kidney disease, lupus nephritis, acute glomerulonephritis, immune nephritis, membranous nephropathy, and IgA nephropathy.

48. The method according to claim 47, wherein the autoimmune disease is membranous nephropathy.

49. The bispecific CAR according to claim 47, wherein the autoimmune disease is IgA nephropathy.

50. The method according to claim 43, wherein the autoimmune disease is multiple sclerosis.

51. The method according to claim 43, wherein the autoimmune disease is scleroderma or systemic sclerosis (SSc).

52. The method according to claim 43, wherein the autoimmune disease is myasthenia gravis.

53-90. (canceled)

91. A method for treating an autoimmune disease comprising administering a nucleic acid molecule, a recombinant vector, or a cell, wherein
the nucleic acid molecule comprises a nucleotide sequence encoding the bispecific CAR according to claim 34;
the recombinant vector encodes the bispecific CAR according to claim 34; or
the cell is an engineered autologous T cell expressing the bispecific CAR according to claim 34.

92. (canceled)

93. The method according to claim 91, wherein the recombinant vector includes or is selected from DNA vectors, RNA vectors, plasmids, liposomes, particles, transposon vectors, CRISPR/Cas9 vectors, lentiviral vectors, or viral vectors.

94. (canceled)

95. The method according to claim 91, wherein the autoimmune disease is a B-cell mediated autoimmune disease.

96. The method according or claim 95, wherein the B-cell mediated autoimmune disease includes one or more systemic lupus erythematosus (SLE), glomerulonephritis includes autoimmune chronic kidney disease, lupus nephritis, acute glomerulonephritis, immune nephritis, membranous nephropathy, IgA nephropathy, Anti-Neutrophil Cytoplasmic Antibody (ANCA) Associated Vasculitis (AAV); scleroderma or systemic sclerosis (SSc); myositis or idiopathic inflammatory myositis including dermatomyositis, polymyositis, immune-mediated necrotizing myopathy (IMNM), antisynthetase syndrome, inclusion body myositis, and overlap myositis; multiple sclerosis (MS); inflammatory bowel disease (IBD); rheumatoid arthritis (RA); Sjogren's syndrome (SS); autoimmune hemolytic anemia; neuromyelitis optica (NMO); neuromyelitis optica spectrum disease (NMOSD); idiopathic thrombocytopenic purpura (ITP); systemic autoimmune small vessel vasculitis syndrome or polyangiitis related to antineutrophil cytoplasmic antibody; Wegener's granulomatosis (GPA), eosinophilic granulomatosis with polyangiitis (EGPA, Churg-Strauss syndrome); pemphigus vulgaris; autoimmune encephalitis; pemphigus vulgaris; myasthenia gravis; antiphospholipid syndrome; Chagas' disease; Graves' disease; polyarteritis nodosa; pulmonary hemorrhage-nephritis syndrome; Kawasaki disease, amyloidosis; monoclonal immunoglobulin of undetermined significance, POEMS syndrome; Crohn's disease; ulcerative colitis; adult onset Still's disease; and chronic progressive cortical demyelinating encephalopathy (CIDP).

97. A drug or preparation, wherein the drug or preparation comprises the nucleic acid molecule, the recombinant vector, or the cell according to claim 91, and optionally a pharmaceutically acceptable carrier, adjuvant or excipient.
